SEQUENCE LISTING (66 sequences)

SEQ ID NO: 1 - DNA, 25 bp, Artificial Sequence
  synthetic
aggattgtttgttacgaggtcgcgt

SEQ ID NO: 2 - DNA, 25 bp, Artificial Sequence
  synthetic
actatactccgcttctctccgcgcc

SEQ ID NO: 3 - DNA, 23 bp, Artificial Sequence
  synthetic
ggtcgcggcgtttgtttagaggc

SEQ ID NO: 4 - DNA, 25 bp, Artificial Sequence
  synthetic
aactcatcctccctcccgaaacgtc

SEQ ID NO: 5 - DNA, 26 bp, Artificial Sequence
  synthetic
cgagtttagtggtttttaggtaacgg

SEQ ID NO: 6 - DNA, 24 bp, Artificial Sequence
  synthetic
tacgaaatccgaaaaaaatccgta

SEQ ID NO: 7 - DNA, 25 bp, Artificial Sequence
  synthetic
tcgaggcggtagtattaggtttacg

SEQ ID NO: 8 - DNA, 25 bp, Artificial Sequence
  synthetic
aatctaaaaaacgaaaatccccgct

SEQ ID NO: 9 - DNA, 26 bp, Artificial Sequence
  synthetic
tttattttgtagcgggaggcgtaggc

SEQ ID NO: 10 - DNA, 25 bp, Artificial Sequence
  synthetic
ctaaactatttctaaccaaaccgca

SEQ ID NO: 11 - DNA, 26 bp, Artificial Sequence
  synthetic
tagtagagcgggtcgggagcgtaagc

SEQ ID NO: 12 - DNA, 31 bp, Artificial Sequence
  synthetic
cgcctactaccctatctaaccgaaaacgaac

SEQ ID NO: 13 - DNA, 25 bp, Artificial Sequence
  synthetic
ttgggaagtttgcggttttttcgtt

SEQ ID NO: 14 - DNA, 30 bp, Artificial Sequence
  synthetic
aaaatccgtaaaaactatcctaaaacgccc

SEQ ID NO: 15 - DNA, 29 bp, Artificial Sequence
  synthetic
aggatgtagtttagttcgtggagttcgag

SEQ ID NO: 16 - DNA, 25 bp, Artificial Sequence
  synthetic
gaaaaacgccaattttacgccgtaa

SEQ ID NO: 17 - DNA, 25 bp, Artificial Sequence
  synthetic
gtgtaatttgggtcgcggttttcgc

SEQ ID NO: 18 - DNA, 26 bp, Artificial Sequence
  synthetic
cgtacctttaacacgcgcgatacgtt

SEQ ID NO: 19 - DNA, 28 bp, Artificial Sequence
  synthetic
ttagggtcgggaaaggattttttatcgt

SEQ ID NO: 20 - DNA, 24 bp, Artificial Sequence
  synthetic
gaaaccgaactcgaaatccacgcg

SEQ ID NO: 21 - DNA, 22 bp, Artificial Sequence
  synthetic
gattttgggttgcggtggtcgt

SEQ ID NO: 22 - DNA, 27 bp, Artificial Sequence
  synthetic
aaacacatataaaaacatttcaacgaa

SEQ ID NO: 23 - DNA, 25 bp, Artificial Sequence
  synthetic
agttatagtttcggaggcgcggagc

SEQ ID NO: 24 - DNA, 25 bp, Artificial Sequence
  synthetic
ccgaaaaacgaaaaaaacaaacgct

SEQ ID NO: 25 - DNA, 31 bp, Artificial Sequence
  synthetic
cgggttatagttataggttggggtatttcgg

SEQ ID NO: 26 - DNA, 20 bp, Artificial Sequence
  synthetic
actctcgccaactccgcgaa

SEQ ID NO: 27 - DNA, 25 bp, Artificial Sequence
  synthetic
gcggttttttggtattaggagtcgt

SEQ ID NO: 28 - DNA, 25 bp, Artificial Sequence
  synthetic
ataaacgtaaccgaattaacccgac

SEQ ID NO: 29 - DNA, 28 bp, Artificial Sequence
  synthetic
ttttattgaagtgggtaaaattttcgag

SEQ ID NO: 30 - DNA, 28 bp, Artificial Sequence
  synthetic
tccgaataaaaaactaaaaacaccgcta

SEQ ID NO: 31 - DNA, 25 bp, Artificial Sequence
  synthetic
ttaggggttagggtaggtcgtgcgt

SEQ ID NO: 32 - DNA, 27 bp, Artificial Sequence
  synthetic
cgcgaaaaacgaaaactaaaaaacgta

SEQ ID NO: 33 - DNA, 27 bp, Artificial Sequence
  synthetic
cgtttgtttaggaaggttgggtttggc

SEQ ID NO: 34 - DNA, 26 bp, Artificial Sequence
  synthetic
gccgtctcgaacgactaaaattcgaa

SEQ ID NO: 35 - DNA, 25 bp, Artificial Sequence
  synthetic
acggttatggaaattggattagcgg

SEQ ID NO: 36 - DNA, 24 bp, Artificial Sequence
  synthetic
ccaaacgaccttaaaaacgccgaa

SEQ ID NO: 37 - DNA, 20 bp, Artificial Sequence
  synthetic
tcggattgcgggaggttgtc

SEQ ID NO: 38 - DNA, 34 bp, Artificial Sequence
  synthetic
cacgtcgaaataatactactccacctaaaaaacg

SEQ ID NO: 39 - DNA, 20 bp, Artificial Sequence
  synthetic
tcgttttagcggaacggcgg

SEQ ID NO: 40 - DNA, 31 bp, Artificial Sequence
  synthetic
gtacgtacatcgaacgaactatacgccgaac

SEQ ID NO: 41 - DNA, 26 bp, Artificial Sequence
  synthetic
ttatagtattaggtggagttgagcgg

SEQ ID NO: 42 - DNA, 25 bp, Artificial Sequence
  synthetic
aacgattcctcgaaaaaaatacgaa

SEQ ID NO: 43 - DNA, 25 bp, Artificial Sequence
  synthetic
gttttgtttggggttttgggttcgg

SEQ ID NO: 44 - DNA, 24 bp, Artificial Sequence
  synthetic
aaacaaaaaccgacaaaactcgct

SEQ ID NO: 45 - DNA, 25 bp, Artificial Sequence
  synthetic
tttaggtgcgttgtagtttagacgg

SEQ ID NO: 46 - DNA, 25 bp, Artificial Sequence
  synthetic
aaacaaatcccaaaaactactcgac

SEQ ID NO: 47 - DNA, 23 bp, Artificial Sequence
  synthetic
ggagaggatttgaagggtttcgt

SEQ ID NO: 48 - DNA, 25 bp, Artificial Sequence
  synthetic
ctctaaaatcctacccaactccgat

SEQ ID NO: 49 - DNA, 25 bp, Artificial Sequence
  synthetic
cgtaggtttaaaagtggttcgcggc

SEQ ID NO: 50 - DNA, 26 bp, Artificial Sequence
  synthetic
ccgattcctatttctattaaaacgaa

SEQ ID NO: 51 - DNA, 27 bp, Artificial Sequence
  synthetic
gggtgttcggtagcggagtattacgtt

SEQ ID NO: 52 - DNA, 25 bp, Artificial Sequence
  synthetic
ataaaaacctccatcgaccccgtcc

SEQ ID NO: 53 - DNA, 24 bp, Artificial Sequence
  synthetic
gcgtgattgatgggtgtattacgt

SEQ ID NO: 54 - DNA, 25 bp, Artificial Sequence
  synthetic
ataaacttccgatccctacaacgaa

SEQ ID NO: 55 - DNA, 22 bp, Artificial Sequence
  synthetic
gtcgaatcgtcggttcgagggc

SEQ ID NO: 56 - DNA, 23 bp, Artificial Sequence
  synthetic
tctccacgattttcgcgaacgct

SEQ ID NO: 57 - DNA, 25 bp, Artificial Sequence
  synthetic
attttgttattgtttcggggatcgt

SEQ ID NO: 58 - DNA, 26 bp, Artificial Sequence
  synthetic
cactcaaaacttatctctcaaacgcc

SEQ ID NO: 59 - DNA, 24 bp, Artificial Sequence
  synthetic
gaggaaagagaagtgggcgttcga

SEQ ID NO: 60 - DNA, 26 bp, Artificial Sequence
  synthetic
cccaatcctaatccacttaacgcgtc

SEQ ID NO: 61 - DNA, 26 bp, Artificial Sequence
  synthetic
ggttggaaaggggtggtttatttcga

SEQ ID NO: 62 - DNA, 23 bp, Artificial Sequence
  synthetic
gaaaacccgcaaaaaaccccgaa

SEQ ID NO: 63 - DNA, 27 bp, Artificial Sequence
  synthetic
gcgttacggaatttaacggtggtagcg

SEQ ID NO: 64 - DNA, 30 bp, Artificial Sequence
  synthetic
cgacgaaaaaaacgcgaacactaaaaaacg

SEQ ID NO: 65 - DNA, 26 bp, Artificial Sequence
  synthetic
tttcgaggatagttcgcgggtttttc

SEQ ID NO: 66 - DNA, 25 bp, Artificial Sequence
  synthetic
attatcgctcgcgtccttaaccgac

(19) United States
(12) Patent Application Publication
(10) Pub. No.: US 2023/0167506 A1
(43) Pub. Date: Jun. 1, 2023

(54) DETECTING PANCREATIC NEUROENDOCRINE TUMORS

(71) Applicant: Mayo Foundation for Medical Education and Research, Rochester, MN (US)

(72) Inventors: David A. Ahlquist, Rochester, MN (US); John B. Kisiel, Rochester, MN (US); William R. Taylor, Lake City, MN (US); Douglas W. Mahoney, Elgin, MN (US); Shounak Majumder, Rochester, MN (US)

(21) Appl. No.: 17/921,710

(22) PCT Filed: May 4, 2021
(86) PCT No.: PCT/US2021/030635

§ 371 Date: Oct. 27, 2022

Related U.S. Application Data
(60) Provisional application No. 63/019,751, filed on May 4, 2020.

(51) Int. Cl. C12Q 1/6886; C12Q 1/6806

(52) U.S. Cl. CPC C12Q 1/6886; C12Q 1/6806; C12Q 2600/154; C12Q 2600/16

(57) ABSTRACT

Provided herein is technology for pancreatic neuroendocrine tumor screening and particularly, but not exclusively, to methods, compositions, and related uses for detecting the presence of pancreatic neuroendocrine tumors.

### CROSS-REFERENCE TO RELATED APPLICATION

The present application claims priority to U.S. Provisional Patent Application No. 63/019,751, filed May 4, 2020, which is hereby incorporated by reference in its entirety.

### FIELD OF INVENTION

Provided herein is technology for pancreatic neuroendocrine tumor screening and particularly, but not exclusively, to methods, compositions, and related uses for detecting the presence of pancreatic neuroendocrine tumors.

### BACKGROUND

Pancreatic neuroendocrine tumors (PNETs) are suspected based on their characteristic radiologic appearance of an enhancing solid pancreatic lesion, and the diagnosis is typically confirmed by EUS-guided biopsy. PNETs can occasionally be cystic and mimic other pancreatic cystic lesions resulting in diagnostic uncertainty. There is currently no blood-based or cyst fluid biomarker for diagnosis of PNETs. An incidentally diagnosed PNET leads to a therapeutic dilemma as pancreatic resections carry significant risk and, although PNETs are typically slow-growing which allows for watchful waiting and periodic surveillance imaging without treatment in selected cases, biological behavior can be unpredictable and size-independent.

Currently, the World Health Organization (WHO) classifies all PNETs into low-grade (G1), intermediate grade (G2), and high grade (G3) categories based upon mitotic count and proliferative index (Ki-67) assessed in pancreatic tissue. There is no non-invasive marker for determining grade and hence there is lack of clear consensus on which patient population is safe to observe. In patients who undergo pancreatic resection, recurrence is not uncommon and can occur several years after surgery. Also, in patients with metastatic disease current pharmacotherapies are tumorostatic and there is no biomarker to monitor disease activity during treatment.

Thus, there is a clinical need for accurate PNET biomarkers that can be applied to both cyst fluid and blood for diagnosis, staging, and surveillance.

The present invention addresses such needs. Indeed, the present invention provides novel methylated DNA markers that discriminate cases of PNET within various biological samples (e.g., tissue, blood).

### SUMMARY

Methylated DNA has been studied as a potential class of biomarkers in the tissues of most tumor types. In many instances, DNA methyltransferases add a methyl group to DNA at cytosine-phosphate-guanine (CpG) island sites as an epigenetic control of gene expression. In a biologically attractive mechanism, acquired methylation events in promoter regions of tumor suppressor genes are thought to silence expression, thus contributing to oncogenesis. DNA methylation may be a more chemically and biologically stable diagnostic tool than RNA or protein expression (Laird (2010) Nat Rev Genet 11: 191-203). Furthermore, in other cancers like sporadic colon cancer, methylation markers offer excellent specificity and are more broadly informative and sensitive than individual DNA mutations (Zou et al (2007) Cancer Epidemiol Biomarkers Prev 16: 2686-96).

Analysis of CpG islands has yielded important findings when applied to animal models and human cell lines. For example, Zhang and colleagues found that amplicons from different parts of the same CpG island may have different levels of methylation (Zhang et al. (2009) PLoS Genet 5: e1000438). Further, methylation levels were distributed bi-modally between highly methylated and unmethylated sequences, further supporting the binary switch-like pattern of DNA methyltransferase activity (Zhang et al. (2009) PLoS Genet 5: e1000438). Analysis of murine tissues in vivo and cell lines in vitro demonstrated that only about 0.3% of high CpG density promoters (HCP, defined as having >7% CpG sequence within a 300 base pair region) were methylated, whereas areas of low CpG density (LCP, defined as having <5% CpG sequence within a 300 base pair region) tended to be frequently methylated in a dynamic tissue-specific pattern (Meissner et al. (2008) Nature 454: 766-70). HCPs include promoters for ubiquitous housekeeping genes and highly regulated developmental genes. Among the HCP sites methylated at >50% were several established markers such as Wnt 2, NDRG2, SFRP2, and BMP3 (Meissner et al. (2008) Nature 454: 766-70).

Epigenetic methylation of DNA at cytosine-phosphate-guanine (CpG) island sites by DNA methyltransferases has been studied as a potential class of biomarkers in the tissues of most tumor types.

Several methods are available to search for novel methylation markers. While micro-array-based interrogation of CpG methylation is a reasonable, high-throughput approach, this strategy is biased towards known regions of interest, mainly established tumor suppressor promotors. Alternative methods for genome-wide analysis of DNA methylation have been developed in the last decade. There are four basic approaches. The first employs digestion of DNA by restriction enzymes which recognize specific methylated sites, followed by several possible analytic techniques which provide methylation data limited to the enzyme recognition site or the primers used to amplify the DNA in quantification steps (such as methylation-specific PCR; MSP). A second approach enriches methylated fractions of genomic DNA using anti-bodies directed to methyl-cytosine or other methylation-specific binding domains followed by microarray analysis or sequencing to map the fragment to a reference genome. This approach does not provide single nucleotide resolution of all methylated sites within the fragment. A third approach begins with bisulfite treatment of the DNA to convert all unmethylated cytosines to uracil, followed by restriction enzyme digestion and complete sequencing of all fragments after coupling to an adapter ligand. The choice of restriction enzymes can enrich the fragments for CpG dense regions, reducing the number of redundant sequences which may map to multiple gene positions during analysis. A fourth approach involves a bisulfite-free treatment of the DNA that describe a bisulfite-free and base-resolution sequencing method, TET-assisted pyridine borane sequencing (TAPS), for non-destructive and direct detection of 5-methylcytosine and 5-hydroxymethylcytosine without affecting unmodified cytosines (see, Liu et al., 2019, Nat Biotechnol. 37, pp. 424-429). In some embodiments, regardless of the specific enzymatic conversion approach, only the methylated cytosines are converted.

Reduced Representation Bisulfite Sequencing (RRBS) yields CpG methylation status data at single nucleotide resolution of 80-90% of all CpG islands and a majority of tumor suppressor promoters at medium to high read coverage. In cancer case—control studies, analysis of these reads results in the identification of differentially methylated regions (DMRs). In previous RRBS analysis of pancreatic cancer specimens, hundreds of DMRs were uncovered, many of which had never been associated with carcinogenesis and many of which were unannotated. Further validation studies on independent tissue sample sets confirmed marker CpGs which were 100% sensitive and specific in terms of performance.

PNETs account for a small but important subset of pancreatic tumors and can present as a solid or cystic pancreatic mass. The prevalence of PNETs in the United States has increased in the last decade largely due to incidental detection with widespread diagnostic use of high definition abdominal imaging (see, Dasari A, et al., JAMA oncology 2017; 3(10):1335-42; Hallet J, et al., Cancer. 2015; 121(4):589-97). The vast majority of PNETs are non-functioning and do not present with a clinical syndrome of hormone overproduction. Despite being clinically silent, PNETs can be histologically high-grade and occasionally manifest with metastatic disease at the time of initial detection, irrespective of size of the primary lesion. There is currently no non-invasive biomarker for accurately detecting PNETs and diagnosis is dependent on tissue sampling which is often challenging in small lesions due to poor diagnostic tissue yield and associated risk of pancreatitis. Also, since NETs can arise in multiple other organs outside the pancreas (lung, small bowel) it would be valuable for a blood-based molecular diagnostic test to localize the site of a primary cancer.

The present invention addresses an important gap in the diagnosis and management of PNETs-namely, the absence of accurate biomarkers. Whole methylome discovery and validation of novel methylated DNA markers (MDMs) was previously accomplished for detecting pancreatic ductal adenocarcinoma (PDAC) in tissue that has led to identification of MDM panels in pancreatic cyst fluid, pancreatic juice and blood that can accurately discriminate PDAC from healthy controls (see, Kisiel J B, et al., Clin Cancer Res. 2015; 21(19):4473-81; Majumder S, Gastroenterology. 150(4):S120-S1; Majumder S, et al., Gastroenterology. 152(5):S148).

Indeed, as described in Example I, experiments conducted during the course for identifying embodiments for the present invention identified a novel set of differentially methylated regions (DMRs) for discriminating PNET derived DNA from non-neoplastic control DNA.

Such experiments list and describe 198 novel DNA methylation markers distinguishing PNET tissue from benign tissue (see, Tables 1A, 1B, 2A, 2B, 4, 5A, and 5C, and Example I).

From these 198 novel DNA methylation markers, further experiments identified the following markers and/or panels of markers capable of distinguishing PNET tissue (e.g., cystic PNET tissue, solid PNET tissue, metastatic PNET tissue) from benign tissue:
ANXA2, CACNA1C_A, CDHR2, FBXL16_B, GP1BB_A, GP1BB_C, HCN2, HPCAL1, LOC100129726, MAX.chr17.77788758-77788971, PDZD2, PTPRN2, RASSF3, RTN2, RUNDC3A, RXRA, SLC38A2, SPTBN4, SRRM3, STX10_B, TMC6_A, TSPO, CUX1, FAM78A, FNBP1, IER2, MOBKL2A, PNMAL2, S1PR4_A, LGALS3, and MYO15B (see, Table 4, 5A and 5C, Example I);
SRRM3, HCN2, SPTBN4, TMC6_A, GP1BB_C, GP1BB_A, STX10_B, CACNA1C_A, CDHR2, PTPRN2, MAX.chr17.77788758.77788971, FBXL16_B, RTN2, HPCAL1, RASSF3, TSPO, RUNDC3A, SLC38A2, MAX.chr19.2478419.2478656, PDZD2, LOC100129726, CUX1, ANXA2, RXRA, S1PR4_A, FNBP1, FAM78A, IER2, PNMAL2, and MOBKL2A (see, Table 5C, Example I); and
SRRM3, HCN2, SPTBN4 and TMC6_A (see, Table 5C, Example I).

From these 198 novel DNA methylation markers, further experiments identified the following markers and/or panels of markers for detecting PNET in blood samples (e.g., plasma samples, whole blood samples, leukocyte samples, serum samples):
ANXA2, CACNA1C_A, CDHR2, FBXL16_B, GP1BB_A, GP1BB_C, HCN2, HPCAL1, LOC100129726, MAX.chr17.77788758-77788971, PDZD2, PTPRN2, RASSF3, RTN2, RUNDC3A, RXRA, SLC38A2, SPTBN4, SRRM3, STX10_B, TMC6_A, TSPO, CUX1, FAM78A, FNBP1, IER2, MOBKL2A, PNMAL2, S1PR4_A, LGALS3, and MYO15B (see, Tables 4, 5A and 5C, Example I); and
SRRM3, HCN2, SPTBN4 and TMC6_A (see, Table 5C, Example I).

From these 198 novel DNA methylation markers, further experiments identified the following markers and/or panels of markers for detecting metastatic PNET in blood samples (e.g., plasma samples, whole blood samples, leukocyte samples, serum samples):
SRRM3, HCN2, SPTBN4, TMC6_A, GP1BB_C, GP1BB_A, STX10_B, CACNA1C_A, CDHR2, PTPRN2, MAX.chr17.77788758.77788971, FBXL16_B, RTN2, HPCAL1, RASSF3, TSPO, RUNDC3A, SLC38A2, MAX.chr19.2478419.2478656, PDZD2, LOC100129726, CUX1, ANXA2, RXRA, S1PR4_A, FNBP1, FAM78A, IER2, PNMAL2, and MOBKL2A (see, Table 5C, Example I).

From these 198 novel DNA methylation markers, further experiments identified the following markers and/or panels of markers for detecting lung neuroendocrine tumor (NET) in blood samples (e.g., plasma samples, whole blood samples, leukocyte samples, serum samples):
SRRM3, HCN2, SPTBN4, TMC6_A, GP1BB_C, GP1BB_A, STX10_B, CACNA1C_A, CDHR2, PTPRN2, MAX.chr17.77788758.77788971, FBXL16_B, RTN2, HPCAL1, RASSF3, TSPO, RUNDC3A, SLC38A2, MAX.chr19.2478419.2478656, PDZD2, LOC100129726, CUX1, ANXA2, RXRA, S1PR4_A, FNBP1, FAM78A, IER2, PNMAL2, and MOBKL2A (see, Table 5C, Example I).

From these 198 novel DNA methylation markers, further experiments identified the following markers and/or panels of markers for detecting small bowel neuroendocrine tumor (NET) in blood samples (e.g., plasma samples, whole blood samples, leukocyte samples, serum samples):
SRRM3, HCN2, SPTBN4, TMC6_A, GP1BB_C, GP1BB_A, STX10_B, CACNA1C_A, CDHR2, PTPRN2, MAX.chr17.77788758.77788971, FBXL16_B, RTN2, HPCAL1, RASSF3, TSPO, RUNDC3A, SLC38A2, MAX.chr19.2478419.2478656, PDZD2, LOC100129726, CUX1, ANXA2, RXRA, S1PR4_A, FNBP1, FAM78A, IER2, PNMAL2, and MOBKL2A (see, Table 5C, Example I).

As described herein, the technology provides a number of methylated DNA markers and subsets thereof (e.g., sets of 2, 3, 4, 5, 6, 7, or 8 markers) with high discrimination for PNET overall and various related NET types (e.g., lung NET, small bowel NET). Experiments applied a selection filter to candidate markers to identify markers that provide a high signal to noise ratio and a low background level to provide high specificity for purposes of PNET screening or diagnosis.

In some embodiments, the technology is related to assessing the presence of and methylation state of one or more of the markers identified herein in a biological sample (e.g., pancreatic tissue sample, blood sample). These markers comprise one or more differentially methylated regions (DMR) as discussed herein, e.g., as provided in Tables 1A and 2A. Methylation state is assessed in embodiments of the technology. As such, the technology provided herein is not restricted in the method by which a gene's methylation state is measured. For example, in some embodiments the methylation state is measured by a genome scanning method. For example, one method involves restriction landmark genomic scanning (Kawai et al. (1994) Mol. Cell. Biol. 14: 7421-7427) and another example involves methylation-sensitive arbitrarily primed PCR (Gonzalgo et al. (1997) Cancer Res. 57: 594-599). In some embodiments, changes in methylation patterns at specific CpG sites are monitored by digestion of genomic DNA with methylation-sensitive restriction enzymes followed by Southern analysis of the regions of interest (digestion-Southern method). In some embodiments, analyzing changes in methylation patterns involves a PCR-based process that involves digestion of genomic DNA with methylation-sensitive restriction enzymes or methylation-dependent restriction enzymes prior to PCR amplification (Singer-Sam et al. (1990) Nucl. Acids Res. 18: 687). In addition, other techniques have been reported that utilize bisulfite treatment of DNA as a starting point for methylation analysis. These include methylation-specific PCR (MSP) (Herman et al. (1992) Proc. Natl. Acad. Sci. USA 93: 9821-9826) and restriction enzyme digestion of PCR products amplified from bisulfite-converted DNA (Sadri and Hornsby (1996) Nucl. Acids Res. 24: 5058-5059; and Xiong and Laird (1997) Nucl. Acids Res. 25: 2532-2534). PCR techniques have been developed for detection of gene mutations (Kuppuswamy et al. (1991) Proc. Natl. Acad. Sci. USA 88: 1143-1147) and quantification of allelic-specific expression (Szabo and Mann (1995) Genes Dev. 9: 3097-3108; and Singer-Sam et al. (1992) PCR Methods Appl. 1: 160-163). Such techniques use internal primers, which anneal to a PCR-generated template and terminate immediately 5′ of the single nucleotide to be assayed. Methods using a “quantitative Ms-SNuPE assay” as described in U.S. Pat. No. 7,037,650 are used in some embodiments.

Upon evaluating a methylation state, the methylation state is often expressed as the fraction or percentage of individual strands of DNA that is methylated at a particular site (e.g., at a single nucleotide, at a particular region or locus, at a longer sequence of interest, e.g., up to a ˜100-bp, 200-bp, 500-bp, 1000-bp subsequence of a DNA or longer) relative to the total population of DNA in the sample comprising that particular site. Traditionally, the amount of the unmethylated nucleic acid is determined by PCR using calibrators. Then, a known amount of DNA is bisulfite treated (or non-bisulfite treated (see, Liu et al., 2019, Nat Biotechnol. 37, pp. 424-429)) and the resulting methylation-specific sequence is determined using either a real-time PCR or other exponential amplification, e.g., a QuARTS assay (e.g., as provided by U.S. Pat. Nos. 8,361,720; 8,715,937; 8,916,344; and 9,212,392).

For example, in some embodiments. methods comprise generating a standard curve for the unmethylated target by using external standards. The standard curve is constructed from at least two points and relates the real-time Ct value for unmethylated DNA to known quantitative standards. Then, a second standard curve for the methylated target is constructed from at least two points and external standards. This second standard curve relates the Ct for methylated DNA to known quantitative standards. Next, the test sample Ct values are determined for the methylated and unmethylated populations and the genomic equivalents of DNA are calculated from the standard curves produced by the first two steps. The percentage of methylation at the site of interest is calculated from the amount of methylated DNAs relative to the total amount of DNAs in the population, e.g., (number of methylated DNAs)/(the number of methylated DNAs+number of unmethylated DNAs)×100.

In some embodiments, the plurality of different target regions comprise a reference target region, and in certain preferred embodiments, the reference target region comprises 3-actin and and/or ZDHHC1, and/or B3GALT6.

Also provided herein are compositions and kits for practicing the methods. For example, in some embodiments, reagents (e.g., primers, probes) specific for one or more MDMs are provided alone or in sets (e.g., sets of primers pairs for amplifying a plurality of markers). Additional reagents for conducting a detection assay may also be provided (e.g., enzymes, buffers, positive and negative controls for conducting QuARTS, PCR, sequencing, bisulfite, Ten-Eleven Translocation (TET) enzyme (e.g., human TET1, human TET2, human TET3, murine TET1, murine TET2, murine TET3, Naegleria TET (NgTET), Coprinopsis cinerea (CcTET)), or a variant thereof), organic borane, or other assays). In some embodiments, the kits contain a reagent capable of modifying DNA in a methylation-specific manner (e.g., a methylation-sensitive restriction enzyme, a methylation-dependent restriction enzyme, Ten-Eleven Translocation (TET) enzyme (e.g., human TET1, human TET2, human TET3, murine TET1, murine TET2, murine TET3, Naegleria TET (NgTET), Coprinopsis cinerea (CcTET)), or a variant thereof), organic borane), and/or an agent capable of detecting an increased level of a protein marker described herein. In some embodiments, the kits containing one or more reagents necessary, sufficient, or useful for conducting a method are provided. Also provided are reactions mixtures containing the reagents. Further provided are master mix reagent sets containing a plurality of reagents that may be added to each other and/or to a test sample to complete a reaction mixture.

In some embodiments, the technology described herein is associated with a programmable machine designed to perform a sequence of arithmetic or logical operations as provided by the methods described herein. For example, some embodiments of the technology are associated with (e.g., implemented in) computer software and/or computer hardware. In one aspect, the technology relates to a computer comprising a form of memory, an element for performing arithmetic and logical operations, and a processing element (e.g., a microprocessor) for executing a series of instructions (e.g., a method as provided herein) to read, manipulate, and store data. In some embodiments, a microprocessor is part of a system for determining a methylation state (e.g., of one or more DMR, e.g., DMR 1-198 as provided in Tables 1A and 2A); comparing methylation states (e.g., of one or more DMR, e.g., DMR 1-198 as provided in Tables 1A and 2A); generating standard curves; determining a Ct value; calculating a fraction, frequency, or percentage of methylation (e.g., of one or more DMR, e.g., DMR 1-198 as provided in Tables 1A and 2A); identifying a CpG island; determining a specificity and/or sensitivity of an assay or marker; calculating an ROC curve and an associated AUC; sequence analysis; all as described herein or is known in the art.

In some embodiments, a microprocessor or computer uses methylation state data in an algorithm to predict a site of a cancer.

In some embodiments, a software or hardware component receives the results of multiple assays and determines a single value result to report to a user that indicates a cancer risk based on the results of the multiple assays (e.g., determining the methylation state of multiple DMR, e.g., as provided in Tables 1B and 2B). Related embodiments calculate a risk factor based on a mathematical combination (e.g., a weighted combination, a linear combination) of the results from multiple assays, e.g., determining the methylation states of multiple markers (such as multiple DMR, e.g., as provided in Tables 1A and 2A). In some embodiments, the methylation state of a DMR defines a dimension and may have values in a multidimensional space and the coordinate defined by the methylation states of multiple DMR is a result, e.g., to report to a user, e.g., related to a cancer risk.

Some embodiments comprise a storage medium and memory components. Memory components (e.g., volatile and/or nonvolatile memory) find use in storing instructions (e.g., an embodiment of a process as provided herein) and/or data (e.g., a work piece such as methylation measurements, sequences, and statistical descriptions associated therewith). Some embodiments relate to systems also comprising one or more of a CPU, a graphics card, and a user interface (e.g., comprising an output device such as display and an input device such as a keyboard).

Programmable machines associated with the technology comprise conventional extant technologies and technologies in development or yet to be developed (e.g., a quantum computer, a chemical computer, a DNA computer, an optical computer, a spintronics based computer, etc.).

In some embodiments, the technology comprises a wired (e.g., metallic cable, fiber optic) or wireless transmission medium for transmitting data. For example, some embodiments relate to data transmission over a network (e.g., a local area network (LAN), a wide area network (WAN), an ad-hoc network, the internet, etc.). In some embodiments, programmable machines are present on such a network as peers and in some embodiments the programmable machines have a client/server relationship.

In some embodiments, data are stored on a computer-readable storage medium such as a hard disk, flash memory, optical media, a floppy disk, etc.

In some embodiments, the technology provided herein is associated with a plurality of programmable devices that operate in concert to perform a method as described herein. For example, in some embodiments, a plurality of computers (e.g., connected by a network) may work in parallel to collect and process data, e.g., in an implementation of cluster computing or grid computing or some other distributed computer architecture that relies on complete computers (with onboard CPUs, storage, power supplies, network interfaces, etc.) connected to a network (private, public, or the internet) by a conventional network interface, such as Ethernet, fiber optic, or by a wireless network technology.

For example, some embodiments provide a computer that includes a computer-readable medium. The embodiment includes a random access memory (RAM) coupled to a processor. The processor executes computer-executable program instructions stored in memory. Such processors may include a microprocessor, an ASIC, a state machine, or other processor, and can be any of a number of computer processors, such as processors from Intel Corporation of Santa Clara, Calif. and Motorola Corporation of Schaumburg, Ill. Such processors include, or may be in communication with, media, for example computer-readable media, which stores instructions that, when executed by the processor, cause the processor to perform the steps described herein.

Embodiments of computer-readable media include, but are not limited to, an electronic, optical, magnetic, or other storage or transmission device capable of providing a processor with computer-readable instructions. Other examples of suitable media include, but are not limited to, a floppy disk, CD-ROM, DVD, magnetic disk, memory chip, ROM, RAM, an ASIC, a configured processor, all optical media, all magnetic tape or other magnetic media, or any other medium from which a computer processor can read instructions. Also, various other forms of computer-readable media may transmit or carry instructions to a computer, including a router, private or public network, or other transmission device or channel, both wired and wireless. The instructions may comprise code from any suitable computer-programming language, including, for example, C, C++, C#, Visual Basic, Java, Python, Perl, and JavaScript.

Computers are connected in some embodiments to a network. Computers may also include a number of external or internal devices such as a mouse, a CD-ROM, DVD, a keyboard, a display, or other input or output devices. Examples of computers are personal computers, digital assistants, personal digital assistants, cellular phones, mobile phones, smart phones, pagers, digital tablets, laptop computers, internet appliances, and other processor-based devices. In general, the computers related to aspects of the technology provided herein may be any type of processor-based platform that operates on any operating system, such as Microsoft Windows, Linux, UNIX, Mac OS X, etc., capable of supporting one or more programs comprising the technology provided herein. Some embodiments comprise a personal computer executing other application programs (e.g., applications). The applications can be contained in memory and can include, for example, a word processing application, a spreadsheet application, an email application, an instant messenger application, a presentation application, an Internet browser application, a calendar/organizer application, and any other application capable of being executed by a client device.

All such components, computers, and systems described herein as associated with the technology may be logical or virtual.

Accordingly, provided herein is technology related to a method of screening for PNET in a sample obtained from a subject, the method comprising assaying a methylation state of a marker in a sample obtained from a subject (e.g., pancreatic tissue) (e.g., a blood sample) and identifying the subject as having PNET when the methylation state of the marker is different than a methylation state of the marker assayed in a subject that does not have PNET, wherein the marker comprises a base in a differentially methylated region (DMR) selected from a group consisting of DMR 1-198 as provided in Tables 1A and 2A.

In some embodiments wherein the sample obtained from the subject is tissue (e.g., pancreatic tissue) and the methylation state of one or more of the following markers is different than a methylation state of the one or more markers assayed in a subject that does not have a PNET indicates the subject has a PNET: ANXA2, CACNA1C_A, CDHR2, FBXL16_B, GP1BB_A, GP1BB_C, HCN2, HPCAL1, LOC100129726, MAX.chr17.77788758-77788971, PDZD2, PTPRN2, RASSF3, RTN2, RUNDC3A, RXRA, SLC38A2, SPTBN4, SRRM3, STX10_B, TMC6_A, TSPO, CUX1, FAM78A, FNBP1, IER2, MOBKL2A, PNMAL2, S1PR4_A, LGALS3, and MYO15B (see, Table 4, 5A and 5C, Example I).

In some embodiments wherein the sample obtained from the subject is a blood sample (e.g., plasma sample, whole blood sample, leukocyte sample, serum sample) and the methylation state of one or more of the following markers is different than a methylation state of the one or more markers assayed in a subject that does not have PNET indicates the subject has PNET: ANXA2, CACNA1C_A, CDHR2, FBXL16_B, GP1BB_A, GP1BB_C, HCN2, HPCAL1, LOC100129726, MAX.chr17.77788758-77788971, PDZD2, PTPRN2, RASSF3, RTN2, RUNDC3A, RXRA, SLC38A2, SPTBN4, SRRM3, STX10_B, TMC6_A, TSPO, CUX1, FAM78A, FNBP1, IER2, MOBKL2A, PNMAL2, S1PR4_A, LGALS3, and MYO15B (see, Tables 4, 5A and 5C, Example I).

In some embodiments wherein the sample obtained from the subject is a blood sample (e.g., plasma sample, whole blood sample, leukocyte sample, serum sample) and the methylation state of one or more of the following markers is different than a methylation state of the one or more markers assayed in a subject that does not have metastatic PNET indicates the subject has metastatic PNET: SRRM3, HCN2, SPTBN4, TMC6_A, GP1BB_C, GP1BB_A, STX10_B, CACNA1C_A, CDHR2, PTPRN2, MAX.chr17.77788758.77788971, FBXL16_B, RTN2, HPCAL1, RASSF3, TSPO, RUNDC3A, SLC38A2, MAX.chr19.2478419.2478656, PDZD2, LOC100129726, CUX1, ANXA2, RXRA, S1PR4_A, FNBP1, FAM78A, IER2, PNMAL2, and MOBKL2A (see, Table 5C, Example I).

In some embodiments wherein the sample obtained from the subject is a blood sample (e.g., plasma sample, whole blood sample, leukocyte sample, serum sample) and the methylation state of one or more of the following markers is different than a methylation state of the one or more markers assayed in a subject that does not have lung NET indicates the subject has lung NET: SRRM3, HCN2, SPTBN4, TMC6_A, GP1BB_C, GP1BB_A, STX10_B, CACNA1C_A, CDHR2, PTPRN2, MAX.chr17.77788758.77788971, FBXL16_B, RTN2, HPCAL1, RASSF3, TSPO, RUNDC3A, SLC38A2, MAX.chr19.2478419.2478656, PDZD2, LOC100129726, CUX1, ANXA2, RXRA, S1PR4_A, FNBP1, FAM78A, IER2, PNMAL2, and MOBKL2A (see, Table 5C, Example I).

In some embodiments wherein the sample obtained from the subject is a blood sample (e.g., plasma sample, whole blood sample, leukocyte sample, serum sample) and the methylation state of one or more of the following markers is different than a methylation state of the one or more markers assayed in a subject that does not have small bowel NET indicates the subject has small bowel NET: SRRM3, HCN2, SPTBN4, TMC6_A, GP1BB_C, GP1BB_A, STX10_B, CACNA1C_A, CDHR2, PTPRN2, MAX.chr17.77788758.77788971, FBXL16_B, RTN2, HPCAL1, RASSF3, TSPO, RUNDC3A, SLC38A2, MAX.chr19.2478419.2478656, PDZD2, LOC100129726, CUX1, ANXA2, RXRA, S1PR4_A, FNBP1, FAM78A, IER2, PNMAL2, and MOBKL2A (see, Table 5C, Example I).

The technology is related to identifying and discriminating PNET and/or various forms of NET (e.g., lung NET, small bowel NET). Some embodiments provide methods comprising assaying a plurality of markers, e.g., comprising assaying 2 to 11 to 100 or 120 or 198 markers (e.g., 1-4, 1-6, 1-7, 1-8, 1-9, 1-10, 1-11, 1-12, 1-13, 1-14, 1-15, 1-16, 1-17, 1-18, 1-19, 1-20, 1-25, 1-50, 1-75, 1-100, 1-150, 1-198) (e.g., 2-4, 2-6, 2-7, 2-8, 2-9, 2-10, 2-11, 2-12, 2-13, 2-14, 2-15, 2-16, 2-17, 2-18, 2-19, 2-20, 2-25, 2-50, 2-75, 2-100, 2-198) (e.g., 3-4, 3-6, 3-7, 3-8, 3-9, 3-10, 3-11, 3-12, 3-13, 3-14, 3-15, 3-16, 3-17, 3-18, 3-19, 3-20, 3-25, 3-50, 3-75, 3-100, 3-198) (e.g., 4-5, 4-6, 4-7, 4-8, 4-9, 4-10, 4-11, 4-12, 4-13, 4-14, 4-15, 4-16, 4-17, 4-18, 4-19, 4-20, 4-25, 4-50, 4-75, 4-100, 4-198) (e.g., 5-6, 5-7, 5-8, 5-9, 5-10, 5-11, 5-12, 5-13, 5-14, 5-15, 5-16, 5-17, 5-18, 5-19, 5-20, 5-25, 5-50, 5-75, 5-100, 5-198).

The technology is not limited in the methylation state assessed. In some embodiments assessing the methylation state of the marker in the sample comprises determining the methylation state of one base. In some embodiments, assaying the methylation state of the marker in the sample comprises determining the extent of methylation at a plurality of bases. Moreover, in some embodiments the methylation state of the marker comprises an increased methylation of the marker relative to a normal methylation state of the marker. In some embodiments, the methylation state of the marker comprises a decreased methylation of the marker relative to a normal methylation state of the marker. In some embodiments the methylation state of the marker comprises a different pattern of methylation of the marker relative to a normal methylation state of the marker.

Furthermore, in some embodiments the marker is a region of 100 or fewer bases, the marker is a region of 500 or fewer bases, the marker is a region of 1000 or fewer bases, the marker is a region of 5000 or fewer bases, or, in some embodiments, the marker is one base. In some embodiments the marker is in a high CpG density promoter.

The technology is not limited by sample type. For example, in some embodiments the sample is a stool sample, a tissue sample (e.g., pancreatic tissue sample), a blood sample (e.g., plasma, leukocyte, serum, whole blood), an excretion, or a urine sample.

Furthermore, the technology is not limited in the method used to determine methylation state. In some embodiments the assaying comprises using methylation specific polymerase chain reaction, nucleic acid sequencing, mass spectrometry, methylation specific nuclease, mass-based separation, or target capture. In some embodiments, the assaying comprises use of a methylation specific oligonucleotide. In some embodiments, the technology uses massively parallel sequencing (e.g., next-generation sequencing) to determine methylation state, e.g., sequencing-by-synthesis, real-time (e.g., single-molecule) sequencing, bead emulsion sequencing, nanopore sequencing, etc.

The technology provides reagents for detecting a DMR, e.g., in some embodiments are provided a set of oligonucleotides comprising the sequences provided by SEQ ID NO: 1-66 (see, Table 3). In some embodiments are provided an oligonucleotide comprising a sequence complementary to a chromosomal region having a base in a DMR, e.g., an oligonucleotide sensitive to methylation state of a DMR.

The technology provides various panels of markers use for identifying PNET, e.g., in some embodiments the marker comprises a chromosomal region having an annotation that is ANXA2, CACNA1C_A, CDHR2, FBXL16_B, GP1BB_A, GP1BB_C, HCN2, HPCAL1, LOC100129726, MAX.chr17.77788758-77788971, PDZD2, PTPRN2, RASSF3, RTN2, RUNDC3A, RXRA, SLC38A2, SPTBN4, SRRM3, STX10_B, TMC6_A, TSPO, CUX1, FAM78A, FNBP1, IER2, MOBKL2A, PNMAL2, S1PR4_A, LGALS3, and MYO15B (see, Table 4, 5A and 5C, Example I).

Kit embodiments are provided, e.g., a kit comprising a reagent capable of modifying DNA in a methylation-specific manner (e.g., a methylation-sensitive restriction enzyme, a methylation-dependent restriction enzyme, Ten Eleven Translocation (TET) enzyme (e.g., human TET1, human TET2, human TET3, murine TET1, murine TET2, murine TET3, Naegleria TET (NgTET), Coprinopsis cinerea (CcTET)), or a variant thereof), organic borane); and a control nucleic acid comprising one or more sequences from DMR 1-198 (from Tables TA and 2A) and having a methylation state associated with a subject who does not have cancer. In some embodiments, kits comprise a bisulfite reagent and an oligonucleotide as described herein. In some embodiments, kits comprise a reagent capable of modifying DNA in a methylation-specific manner (e.g., a methylation-sensitive restriction enzyme, a methylation-dependent restriction enzyme, Ten Eleven Translocation (TET) enzyme (e.g., human TET1, human TET2, human TET3, murine TET1, murine TET2, murine TET3, Naegleria TET (NgTET), Coprinopsis cinerea (CcTET)), or a variant thereof), organic borane); and a control nucleic acid comprising one or more sequences from DMR 1-198 (from Tables TA and 2A) and having a methylation state associated with a subject who has a specific type of cancer. Some kit embodiments comprise a sample collector for obtaining a sample from a subject (e.g., a stool sample; tissue sample; plasma sample, serum sample, whole blood sample); a reagent capable of modifying DNA in a methylation-specific manner (e.g., a methylation-sensitive restriction enzyme, a methylation-dependent restriction enzyme, Ten Eleven Translocation (TET) enzyme (e.g., human TET1, human TET2, human TET3, murine TET1, murine TET2, murine TET3, Naegleria TET (NgTET), Coprinopsis cinerea (CcTET)), or a variant thereof), organic borane); and an oligonucleotide as described herein.

The technology is related to embodiments of compositions (e.g., reaction mixtures). In some embodiments are provided a composition comprising a nucleic acid comprising a DMR and a reagent capable of modifying DNA in a methylation-specific manner (e.g., a methylation-sensitive restriction enzyme, a methylation-dependent restriction enzyme, Ten Eleven Translocation (TET) enzyme (e.g., human TET1, human TET2, human TET3, murine TET1, murine TET2, murine TET3, Naegleria TET (NgTET), Coprinopsis cinerea (CcTET)), or a variant thereof), organic borane). Some embodiments provide a composition comprising a nucleic acid comprising a DMR and an oligonucleotide as described herein. Some embodiments provide a composition comprising a nucleic acid comprising a DMR and a methylation-sensitive restriction enzyme. Some embodiments provide a composition comprising a nucleic acid comprising a DMR and a polymerase.

Additional related method embodiments are provided for screening for PNET in a sample obtained from a subject (e.g., pancreatic tissue sample; blood sample; stool sample), e.g., a method comprising determining a methylation state of a marker in the sample comprising a base in a DMR that is one or more of DMR 1-198 (from Tables 1A and 2A); comparing the methylation state of the marker from the subject sample to a methylation state of the marker from a normal control sample from a subject who does not have PNET; and determining a confidence interval and/or a p value of the difference in the methylation state of the subject sample and the normal control sample. In some embodiments, the confidence interval is 90%, 95%, 97.5%, 98%, 99%, 99.5%, 99.9% or 99.99% and the p value is 0.1, 0.05, 0.025, 0.02, 0.01, 0.005, 0.001, or 0.0001. Some embodiments of methods provide steps of reacting a nucleic acid comprising a DMR with a reagent capable of modifying nucleic acid in a methylation-specific manner (e.g., a methylation-sensitive restriction enzyme, a methylation-dependent restriction enzyme, Ten Eleven Translocation (TET) enzyme (e.g., human TET1, human TET2, human TET3, murine TET1, murine TET2, murine TET3, Naegleria TET (NgTET), Coprinopsis cinerea (CcTET)), or a variant thereof), organic borane) to produce, for example, nucleic acid modified in a methylation-specific manner; sequencing the nucleic acid modified in a methylation-specific manner to provide a nucleotide sequence of the nucleic acid modified in a methylation-specific manner; comparing the nucleotide sequence of the nucleic acid modified in a methylation-specific manner with a nucleotide sequence of a nucleic acid comprising the DMR from a subject who does not have a specific type of cancer to identify differences in the two sequences; and identifying the subject as having PNET (e.g., PNET and/or a form of NET: lung NET, small bowel NET) when a difference is present.

Systems for screening for PNET in a sample obtained from a subject are provided by the technology. Exemplary embodiments of systems include, e.g., a system for screening for PNET and/or related NET types (e.g., lung NET, small bowel NET) in a sample obtained from a subject (e.g., pancreatic tissue sample; plasma sample; stool sample), the system comprising an analysis component configured to determine the methylation state of a sample, a software component configured to compare the methylation state of the sample with a control sample or a reference sample methylation state recorded in a database, and an alert component configured to alert a user of a PNET-associated methylation state. An alert is determined in some embodiments by a software component that receives the results from multiple assays (e.g., determining the methylation states of multiple markers, e.g., DMR, e.g., as provided in Tables 1A and 2A) and calculating a value or result to report based on the multiple results. Some embodiments provide a database of weighted parameters associated with each DMR provided herein for use in calculating a value or result and/or an alert to report to a user (e.g., such as a physician, nurse, clinician, etc.). In some embodiments all results from multiple assays are reported and in some embodiments one or more results are used to provide a score, value, or result based on a composite of one or more results from multiple assays that is indicative of a cancer risk in a subject.

In some embodiments of systems, a sample comprises a nucleic acid comprising a DMR. In some embodiments the system further comprises a component for isolating a nucleic acid, a component for collecting a sample such as a component for collecting a stool sample. In some embodiments, the system comprises nucleic acid sequences comprising a DMR. In some embodiments the database comprises nucleic acid sequences from subjects who do not have PNET and/or a related NET type (e.g., lung NET, small bowel NET). Also provided are nucleic acids, e.g., a set of nucleic acids, each nucleic acid having a sequence comprising a DMR. In some embodiments the set of nucleic acids wherein each nucleic acid has a sequence from a subject who does not have PNET and/or a related NET type (e.g., lung NET, small bowel NET). Related system embodiments comprise a set of nucleic acids as described and a database of nucleic acid sequences associated with the set of nucleic acids. Some embodiments further comprise a reagent capable of modifying DNA in a methylation-specific manner (e.g., a methylation-sensitive restriction enzyme, a methylation-dependent restriction enzyme, Ten Eleven Translocation (TET) enzyme (e.g., human TET1, human TET2, human TET3, murine TET1, murine TET2, murine TET3, Naegleria TET (NgTET), Coprinopsis cinerea (CcTET)), or a variant thereof), organic borane). Some embodiments further comprise a nucleic acid sequencer.

In certain embodiments, methods for characterizing a sample (e.g., pancreatic tissue sample; blood sample; stool sample) from a human patient are provided. For example, in some embodiments such embodiments comprise obtaining DNA from a sample of a human patient; assaying a methylation state of a DNA methylation marker comprising a base in a differentially methylated region (DMR) selected from a group consisting of DMR 1-198 from Tables TA and 2A; and comparing the assayed methylation state of the one or more DNA methylation markers with methylation level references for the one or more DNA methylation markers for human patients not having PNET and/or a related NET type (e.g., lung NET, small bowel NET).

Such methods are not limited to a particular type of sample from a human patient. In some embodiments, the sample is a pancreatic tissue sample. In some embodiments, the sample is a plasma sample. In some embodiments, the sample is a stool sample, a tissue sample, a pancreatic tissue sample, a blood sample (e.g., leukocyte sample, plasma sample, whole blood sample, serum sample), or a urine sample.

In some embodiments, such methods comprise assaying a plurality of DNA methylation markers (e.g., 1-4, 1-6, 1-7, 1-8, 1-9, 1-10, 1-11, 1-12, 1-13, 1-14, 1-15, 1-16, 1-17, 1-18, 1-19, 1-20, 1-25, 1-50, 1-75, 1-100, 1-150, 1-198) (e.g., 2-4, 2-6, 2-7, 2-8, 2-9, 2-10, 2-11, 2-12, 2-13, 2-14, 2-15, 2-16, 2-17, 2-18, 2-19, 2-20, 2-25, 2-50, 2-75, 2-100, 2-198) (e.g., 3-4, 3-6, 3-7, 3-8, 3-9, 3-10, 3-11, 3-12, 3-13, 3-14, 3-15, 3-16, 3-17, 3-18, 3-19, 3-20, 3-25, 3-50, 3-75, 3-100, 3-198) (e.g., 4-5, 4-6, 4-7, 4-8, 4-9, 4-10, 4-11, 4-12, 4-13, 4-14, 4-15, 4-16, 4-17, 4-18, 4-19, 4-20, 4-25, 4-50, 4-75, 4-100, 4-198) (e.g., 5-6, 5-7, 5-8, 5-9, 5-10, 5-11, 5-12, 5-13, 5-14, 5-15, 5-16, 5-17, 5-18, 5-19, 5-20, 5-25, 5-50, 5-75, 5-100, 5-198). In some embodiments, such methods comprise assaying 2 to 11 DNA methylation markers. In some embodiments, such methods comprise assaying 12 to 120 DNA methylation markers. In some embodiments, such methods comprise assaying 2 to 198 DNA methylation markers. In some embodiments, such methods comprise assaying the methylation state of the one or more DNA methylation markers in the sample comprises determining the methylation state of one base. In some embodiments, such methods comprise assaying the methylation state of the one or more DNA methylation markers in the sample comprises determining the extent of methylation at a plurality of bases. In some embodiments, such methods comprise assaying a methylation state of a forward strand or assaying a methylation state of a reverse strand.

In some embodiments, the DNA methylation marker is a region of 100 or fewer bases. In some embodiments, the DNA methylation marker is a region of 500 or fewer bases. In some embodiments, the DNA methylation marker is a region of 1000 or fewer bases. In some embodiments, the DNA methylation marker is a region of 5000 or fewer bases. In some embodiments, the DNA methylation marker is one base. In some embodiments, the DNA methylation marker is in a high CpG density promoter.

In some embodiments, the assaying comprises using methylation specific polymerase chain reaction, nucleic acid sequencing, mass spectrometry, methylation specific nuclease, mass-based separation, or target capture.

In some embodiments, the assaying comprises use of a methylation specific oligonucleotide. In some embodiments, the methylation specific oligonucleotide is selected from the group consisting of SEQ ID NO: 1-66 (Table 3).

In some embodiments, a chromosomal region having an annotation selected from the group consisting of ANXA2, CACNA1C_A, CDHR2, FBXL16_B, GP1BB_A, GP1BB_C, HCN2, HPCAL1, LOC100129726, MAX.chr17.77788758-77788971, PDZD2, PTPRN2, RASSF3, RTN2, RUNDC3A, RXRA, SLC38A2, SPTBN4, SRRM3, STX10_B, TMC6_A, TSPO, CUX1, FAM78A, FNBP1, IER2, MOBKL2A, PNMAL2, S1PR4_A, LGALS3, and MYO15B (see, Table 4, 5A and 5C, Example I) comprises the DNA methylation marker.

In some embodiments, such methods comprise determining the methylation state of two DNA methylation markers. In some embodiments, such methods comprise determining the methylation state of a pair of DNA methylation markers provided in a row of Tables 1A and 2A.

In certain embodiments, the technology provides methods for characterizing a sample (e.g., pancreatic tissue sample; leukocyte sample; plasma sample; whole blood sample; serum sample; stool sample) obtained from a human patient. In some embodiments, such methods comprise determining a methylation state of a DNA methylation marker in the sample comprising a base in a DMR selected from a group consisting of DMR 1-198 from Tables 1A and 2A; comparing the methylation state of the DNA methylation marker from the patient sample to a methylation state of the DNA methylation marker from a normal control sample from a human subject who does not have a PNET and/or a related NET type (e.g., lung NET, small bowel NET); and determining a confidence interval and/or a p value of the difference in the methylation state of the human patient and the normal control sample. In some embodiments, the confidence interval is 90%, 95%, 97.5%, 98%, 99%, 99.5%, 99.9% or 99.99% and the p value is 0.1, 0.05, 0.025, 0.02, 0.01, 0.005, 0.001, or 0.0001.

In certain embodiments, the technology provides methods for characterizing a sample obtained from a human subject (e.g., pancreatic tissue sample; leukocyte sample; plasma sample; whole blood sample; serum sample; stool sample), the method comprising reacting a nucleic acid comprising a DMR with a reagent capable of modifying DNA in a methylation-specific manner (e.g., a methylation-sensitive restriction enzyme, a methylation-dependent restriction enzyme, and a bisulfite reagent) to produce nucleic acid modified in a methylation-specific manner; sequencing the nucleic acid modified in a methylation-specific manner to provide a nucleotide sequence of the nucleic acid modified in a methylation-specific manner; comparing the nucleotide sequence of the nucleic acid modified in a methylation-specific manner with a nucleotide sequence of a nucleic acid comprising the DMR from a subject who does not have PNET to identify differences in the two sequences.

In certain embodiments, the technology provides systems for characterizing a sample obtained from a human subject (e.g., pancreatic tissue sample; plasma sample; stool sample), the system comprising an analysis component configured to determine the methylation state of a sample, a software component configured to compare the methylation state of the sample with a control sample or a reference sample methylation state recorded in a database, and an alert component configured to determine a single value based on a combination of methylation states and alert a user of a PNET-associated methylation state. In some embodiments, the sample comprises a nucleic acid comprising a DMR.

In some embodiments, such systems further comprise a component for isolating a nucleic acid. In some embodiments, such systems further comprise a component for collecting a sample.

In some embodiments, the sample is a stool sample, a tissue sample, a pancreatic tissue sample, a blood sample (e.g., plasma sample, leukocyte sample, whole blood sample, serum sample), or a urine sample.

In some embodiments, the database comprises nucleic acid sequences comprising a DMR. In some embodiments, the database comprises nucleic acid sequences from subjects who do not have PNET.

Additional embodiments will be apparent to persons skilled in the relevant art based on the teachings contained herein.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.” The transitional phrase “consisting essentially of” as used in claims in the present application limits the scope of a claim to the specified materials or steps “and those that do not materially affect the basic and novel characteristic(s)” of the claimed invention, as discussed in In re Herz, 537 F.2d 549, 551-52, 190 USPQ 461, 463 (CCPA 1976). For example, a composition “consisting essentially of” recited elements may contain an unrecited contaminant at a level such that, though present, the contaminant does not alter the function of the recited composition as compared to a pure composition, i.e., a composition “consisting of” the recited components.

As used herein, a “nucleic acid” or “nucleic acid molecule” generally refers to any ribonucleic acid or deoxyribonucleic acid, which may be unmodified or modified DNA or RNA. “Nucleic acids” include, without limitation, single- and double-stranded nucleic acids. As used herein, the term “nucleic acid” also includes DNA as described above that contains one or more modified bases. Thus, DNA with a backbone modified for stability or for other reasons is a “nucleic acid”. The term “nucleic acid” as it is used herein embraces such chemically, enzymatically, or metabolically modified forms of nucleic acids, as well as the chemical forms of DNA characteristic of viruses and cells, including for example, simple and complex cells.

The terms “oligonucleotide” or “polynucleotide” or “nucleotide” or “nucleic acid” refer to a molecule having two or more deoxyribonucleotides or ribonucleotides, preferably more than three, and usually more than ten. The exact size will depend on many factors, which in turn depends on the ultimate function or use of the oligonucleotide. The oligonucleotide may be generated in any manner, including chemical synthesis, DNA replication, reverse transcription, or a combination thereof. Typical deoxyribonucleotides for DNA are thymine, adenine, cytosine, and guanine. Typical ribonucleotides for RNA are uracil, adenine, cytosine, and guanine.

As used herein, the terms “locus” or “region” of a nucleic acid refer to a subregion of a nucleic acid, e.g., a gene on a chromosome, a single nucleotide, a CpG island, etc.

The terms “complementary” and “complementarity” refer to nucleotides (e.g., 1 nucleotide) or polynucleotides (e.g., a sequence of nucleotides) related by the base-pairing rules. For example, the sequence 5′-A-G-T-3′ is complementary to the sequence 3′-T-C-A-5′. Complementarity may be “partial,” in which only some of the nucleic acids' bases are matched according to the base pairing rules. Or, there may be “complete” or “total” complementarity between the nucleic acids. The degree of complementarity between nucleic acid strands effects the efficiency and strength of hybridization between nucleic acid strands. This is of particular importance in amplification reactions and in detection methods that depend upon binding between nucleic acids.

The term “gene” refers to a nucleic acid (e.g., DNA or RNA) sequence that comprises coding sequences necessary for the production of an RNA, or of a polypeptide or its precursor. A functional polypeptide can be encoded by a full length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a gene” may comprise fragments of the gene or the entire gene.

The term “gene” also encompasses the coding regions of a structural gene and includes sequences located adjacent to the coding region on both the 5′ and 3′ ends, e.g., for a distance of about 1 kb on either end, such that the gene corresponds to the length of the full-length mRNA (e.g., comprising coding, regulatory, structural and other sequences). The sequences that are located 5′ of the coding region and that are present on the mRNA are referred to as 5′ non-translated or untranslated sequences. The sequences that are located 3′ or downstream of the coding region and that are present on the mRNA are referred to as 3′ non-translated or 3′ untranslated sequences. The term “gene” encompasses both cDNA and genomic forms of a gene. In some organisms (e.g., eukaryotes), a genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ ends of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, posttranscriptional cleavage, and poly adenylation.

The term “wild-type” when made in reference to a gene refers to a gene that has the characteristics of a gene isolated from a naturally occurring source. The term “wild-type” when made in reference to a gene product refers to a gene product that has the characteristics of a gene product isolated from a naturally occurring source. The term “naturally-occurring” as applied to an object refers to the fact that an object can be found in nature. For example, a polypeptide or polynucleotide sequence that is present in an organism (including viruses) that can be isolated from a source in nature and which has not been intentionally modified by the hand of a person in the laboratory is naturally-occurring. A wild-type gene is often that gene or allele that is most frequently observed in a population and is thus arbitrarily designated the “normal” or “wild-type” form of the gene. In contrast, the term “modified” or “mutant” when made in reference to a gene or to a gene product refers, respectively, to a gene or to a gene product that displays modifications in sequence and/or functional properties (e.g., altered characteristics) when compared to the wild-type gene or gene product. It is noted that naturally-occurring mutants can be isolated; these are identified by the fact that they have altered characteristics when compared to the wild-type gene or gene product.

The term “allele” refers to a variation of a gene; the variations include but are not limited to variants and mutants, polymorphic loci, and single nucleotide polymorphic loci, frameshift, and splice mutations. An allele may occur naturally in a population or it might arise during the lifetime of any particular individual of the population.

Thus, the terms “variant” and “mutant” when used in reference to a nucleotide sequence refer to a nucleic acid sequence that differs by one or more nucleotides from another, usually related, nucleotide acid sequence. A “variation” is a difference between two different nucleotide sequences; typically, one sequence is a reference sequence.

“Amplification” is a special case of nucleic acid replication involving template specificity. It is to be contrasted with non-specific template replication (e.g., replication that is template-dependent but not dependent on a specific template). Template specificity is here distinguished from fidelity of replication (e.g., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

The term “amplifying” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR; see, e.g., U.S. Pat. No. 5,494,810; herein incorporated by reference in its entirety) are forms of amplification. Additional types of amplification include, but are not limited to, allele-specific PCR (see, e.g., U.S. Pat. No. 5,639,611; herein incorporated by reference in its entirety), assembly PCR (see, e.g., U.S. Pat. No. 5,965,408; herein incorporated by reference in its entirety), helicase-dependent amplification (see, e.g., U.S. Pat. No. 7,662,594; herein incorporated by reference in its entirety), hot-start PCR (see, e.g., U.S. Pat. Nos. 5,773,258 and 5,338,671; each herein incorporated by reference in their entireties), intersequence-specific PCR, inverse PCR (see, e.g., Triglia, et al. (1988) Nucleic Acids Res., 16:8186; herein incorporated by reference in its entirety), ligation-mediated PCR (see, e.g., Guilfoyle, R. et al., Nucleic Acids Research, 25:1854-1858 (1997); U.S. Pat. No. 5,508,169; each of which are herein incorporated by reference in their entireties), methylation-specific PCR (see, e.g., Herman, et al., (1996) PNAS 93(13) 9821-9826; herein incorporated by reference in its entirety), miniprimer PCR, multiplex ligation-dependent probe amplification (see, e.g., Schouten, et al., (2002) Nucleic Acids Research 30(12): e57; herein incorporated by reference in its entirety), multiplex PCR (see, e.g., Chamberlain, et al., (1988) Nucleic Acids Research 16(23) 11141-11156; Ballabio, et al., (1990) Human Genetics 84(6) 571-573; Hayden, et al., (2008) BMC Genetics 9:80; each of which are herein incorporated by reference in their entireties), nested PCR, overlap-extension PCR (see, e.g., Higuchi, et al., (1988) Nucleic Acids Research 16(15) 7351-7367; herein incorporated by reference in its entirety), real time PCR (see, e.g., Higuchi, et al., (1992) Biotechnology 10:413-417; Higuchi, et al., (1993) Biotechnology 11:1026-1030; each of which are herein incorporated by reference in their entireties), reverse transcription PCR (see, e.g., Bustin, S. A. (2000) J. Molecular Endocrinology 25:169-193; herein incorporated by reference in its entirety), solid phase PCR, thermal asymmetric interlaced PCR, and Touchdown PCR (see, e.g., Don, et al., Nucleic Acids Research (1991) 19(14) 4008; Roux, K. (1994) Biotechniques 16(5) 812-814; Hecker, et al., (1996) Biotechniques 20(3) 478-485; each of which are herein incorporated by reference in their entireties). Polynucleotide amplification also can be accomplished using digital PCR (see, e.g., Kalinina, et al., Nucleic Acids Research. 25; 1999-2004, (1997); Vogelstein and Kinzler, Proc Natl Acad Sci USA. 96; 9236-41, (1999); International Patent Publication No. WO05023091A2; US Patent Application Publication No. 20070202525; each of which are incorporated herein by reference in their entireties).

The term “polymerase chain reaction” (“PCR”) refers to the method of K. B. Mullis U.S. Pat. Nos. 4,683,195, 4,683,202, and 4,965,188, that describe a method for increasing the concentration of a segment of a target sequence in a mixture of genomic or other DNA or RNA, without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (“PCR”). Because the desired amplified segments of the target sequence become the predominant sequences (in terms of concentration) in the mixture, they are said to be “PCR amplified” and are “PCR products” or “amplicons.” Those of skill in the art will understand the term “PCR” encompasses many variants of the originally described method using, e.g., real time PCR, nested PCR, reverse transcription PCR (RT-PCR), single primer and arbitrarily primed PCR, etc.

Template specificity is achieved in most amplification techniques by the choice of enzyme. Amplification enzymes are enzymes that, under conditions they are used, will process only specific sequences of nucleic acid in a heterogeneous mixture of nucleic acid. For example, in the case of Q-beta replicase, MDV-1 RNA is the specific template for the replicase (Kacian et al., Proc. Natl. Acad. Sci. USA, 69:3038 [1972]). Other nucleic acid will not be replicated by this amplification enzyme. Similarly, in the case of T7 RNA polymerase, this amplification enzyme has a stringent specificity for its own promoters (Chamberlin et al, Nature, 228:227 [1970]). In the case of T4 DNA ligase, the enzyme will not ligate the two oligonucleotides or polynucleotides, where there is a mismatch between the oligonucleotide or polynucleotide substrate and the template at the ligation junction (Wu and Wallace (1989) Genomics 4:560). Finally, thermostable template-dependent DNA polymerases (e.g., Taq and Pfu DNA polymerases), by virtue of their ability to function at high temperature, are found to display high specificity for the sequences bounded and thus defined by the primers; the high temperature results in thermodynamic conditions that favor primer hybridization with the target sequences and not hybridization with non-target sequences (H. A. Erlich (ed.), PCR Technology, Stockton Press [1989]).

As used herein, the term “nucleic acid detection assay” refers to any method of determining the nucleotide composition of a nucleic acid of interest. Nucleic acid detection assay include but are not limited to, DNA sequencing methods, probe hybridization methods, structure specific cleavage assays (e.g., the INVADER assay, (Hologic, Inc.) and are described, e.g., in U.S. Pat. Nos. 5,846,717, 5,985,557, 5,994,069, 6,001,567, 6,090,543, and 6,872,816; Lyamichev et al., Nat. Biotech., 17:292 (1999), Hall et al., PNAS, USA, 97:8272 (2000), and U.S. Pat. No. 9,096,893, each of which is herein incorporated by reference in its entirety for all purposes); enzyme mismatch cleavage methods (e.g., Variagenics, U.S. Pat. Nos. 6,110,684, 5,958,692, 5,851,770, herein incorporated by reference in their entireties); polymerase chain reaction (PCR), described above; branched hybridization methods (e.g., Chiron, U.S. Pat. Nos. 5,849,481, 5,710,264, 5,124,246, and 5,624,802, herein incorporated by reference in their entireties); rolling circle replication (e.g., U.S. Pat. Nos. 6,210,884, 6,183,960 and 6,235,502, herein incorporated by reference in their entireties); NASBA (e.g., U.S. Pat. No. 5,409,818, herein incorporated by reference in its entirety); molecular beacon technology (e.g., U.S. Pat. No. 6,150,097, herein incorporated by reference in its entirety); E-sensor technology (Motorola, U.S. Pat. Nos. 6,248,229, 6,221,583, 6,013,170, and 6,063,573, herein incorporated by reference in their entireties); cycling probe technology (e.g., U.S. Pat. Nos. 5,403,711, 5,011,769, and 5,660,988, herein incorporated by reference in their entireties); Dade Behring signal amplification methods (e.g., U.S. Pat. Nos. 6,121,001, 6,110,677, 5,914,230, 5,882,867, and 5,792,614, herein incorporated by reference in their entireties); ligase chain reaction (e.g., Baranay Proc. Natl. Acad. Sci USA 88, 189-93 (1991)); and sandwich hybridization methods (e.g., U.S. Pat. No. 5,288,609, herein incorporated by reference in its entirety).

The term “amplifiable nucleic acid” refers to a nucleic acid that may be amplified by any amplification method. It is contemplated that “amplifiable nucleic acid” will usually comprise “sample template.”

The term “sample template” refers to nucleic acid originating from a sample that is analyzed for the presence of “target” (defined below). In contrast, “background template” is used in reference to nucleic acid other than sample template that may or may not be present in a sample. Background template is most often inadvertent. It may be the result of carryover or it may be due to the presence of nucleic acid contaminants sought to be purified away from the sample. For example, nucleic acids from organisms other than those to be detected may be present as background in a test sample.

The term “primer” refers to an oligonucleotide, whether occurring naturally as, e.g., a nucleic acid fragment from a restriction digest, or produced synthetically, that is capable of acting as a point of initiation of synthesis when placed under conditions in which synthesis of a primer extension product that is complementary to a nucleic acid template strand is induced, (e.g., in the presence of nucleotides and an inducing agent such as a DNA polymerase, and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer, and the use of the method.

The term “probe” refers to an oligonucleotide (e.g., a sequence of nucleotides), whether occurring naturally as in a purified restriction digest or produced synthetically, recombinantly, or by PCR amplification, that is capable of hybridizing to another oligonucleotide of interest. A probe may be single-stranded or double-stranded. Probes are useful in the detection, identification, and isolation of particular gene sequences (e.g., a “capture probe”). It is contemplated that any probe used in the present invention may, in some embodiments, be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

The term “target,” as used herein refers to a nucleic acid sought to be sorted out from other nucleic acids, e.g., by probe binding, amplification, isolation, capture, etc. For example, when used in reference to the polymerase chain reaction, “target” refers to the region of nucleic acid bounded by the primers used for polymerase chain reaction, while when used in an assay in which target DNA is not amplified, e.g., in some embodiments of an invasive cleavage assay, a target comprises the site at which a probe and invasive oligonucleotides (e.g., INVADER oligonucleotide) bind to form an invasive cleavage structure, such that the presence of the target nucleic acid can be detected. A “segment” is defined as a region of nucleic acid within the target sequence.

Accordingly, as used herein, “non-target”, e.g., as it is used to describe a nucleic acid such as a DNA, refers to nucleic acid that may be present in a reaction, but that is not the subject of detection or characterization by the reaction. In some embodiments, non-target nucleic acid may refer to nucleic acid present in a sample that does not, e.g., contain a target sequence, while in some embodiments, non-target may refer to exogenous nucleic acid, i.e., nucleic acid that does not originate from a sample containing or suspected of containing a target nucleic acid, and that is added to a reaction, e.g., to normalize the activity of an enzyme (e.g., polymerase) to reduce variability in the performance of the enzyme in the reaction. As used herein, “methylation” refers to cytosine methylation at positions C5 or N4 of cytosine, the N6 position of adenine, or other types of nucleic acid methylation. In vitro amplified DNA is usually unmethylated because typical in vitro DNA amplification methods do not retain the methylation pattern of the amplification template. However, “unmethylated DNA” or “methylated DNA” can also refer to amplified DNA whose original template was unmethylated or methylated, respectively.

As used herein, the term “amplification reagents” refers to those reagents (deoxyribonucleoside triphosphates, buffer, etc.), needed for amplification except for primers, nucleic acid template, and the amplification enzyme. Typically, amplification reagents along with other reaction components are placed and contained in a reaction vessel.

As used herein, the term “control” when used in reference to nucleic acid detection or analysis refers to a nucleic acid having known features (e.g., known sequence, known copy-number per cell), for use in comparison to an experimental target (e.g., a nucleic acid of unknown concentration). A control may be an endogenous, preferably invariant gene against which a test or target nucleic acid in an assay can be normalized. Such normalizing controls for sample-to-sample variations that may occur in, for example, sample processing, assay efficiency, etc., and allows accurate sample-to-sample data comparison. Genes that find use for normalizing nucleic acid detection assays on human samples include, e.g., β-actin, ZDHHC1, and B3GALT6 (see, e.g., U.S. patent application Ser. Nos 14/966,617 and 62/364,082, each incorporated herein by reference.

Controls may also be external. For example, in quantitative assays such as qPCR, QuARTS, etc., a “calibrator” or “calibration control” is a nucleic acid of known sequence, e.g., having the same sequence as a portion of an experimental target nucleic acid, and a known concentration or series of concentrations (e.g., a serially diluted control target for generation of calibration curved in quantitative PCR). Typically, calibration controls are analyzed using the same reagents and reaction conditions as are used on an experimental DNA. In certain embodiments, the measurement of the calibrators is done at the same time, e.g., in the same thermal cycler, as the experimental assay. In preferred embodiments, multiple calibrators may be included in a single plasmid, such that the different calibrator sequences are easily provided in equimolar amounts. In particularly preferred embodiments, plasmid calibrators are digested, e.g., with one or more restriction enzymes, to release calibrator portion from the plasmid vector. See, e.g., WO 2015/066695, which is included herein by reference.

As used herein “ZDHHC1” refers to a gene encoding a protein characterized as a zinc finger, DHHC-type containing 1, located in human DNA on Chr 16 (16q22.1) and belonging to the DHHC palmitoyltransferase family. As used herein, “methylation” refers to cytosine methylation at positions C5 or N4 of cytosine, the N6 position of adenine, or other types of nucleic acid methylation. In vitro amplified DNA is usually unmethylated because typical in vitro DNA amplification methods do not retain the methylation pattern of the amplification template. However, “unmethylated DNA” or “methylated DNA” can also refer to amplified DNA whose original template was unmethylated or methylated, respectively.

As used herein, “methylation” refers to cytosine methylation at positions C5 or N4 of cytosine, the N6 position of adenine, or other types of nucleic acid methylation. In vitro amplified DNA is usually unmethylated because typical in vitro DNA amplification methods do not retain the methylation pattern of the amplification template. However, “unmethylated DNA” or “methylated DNA” can also refer to amplified DNA whose original template was unmethylated or methylated, respectively.

Accordingly, as used herein a “methylated nucleotide” or a “methylated nucleotide base” refers to the presence of a methyl moiety on a nucleotide base, where the methyl moiety is not present in a recognized typical nucleotide base. For example, cytosine does not contain a methyl moiety on its pyrimidine ring, but 5-methylcytosine contains a methyl moiety at position 5 of its pyrimidine ring. Therefore, cytosine is not a methylated nucleotide and 5-methylcytosine is a methylated nucleotide. In another example, thymine contains a methyl moiety at position 5 of its pyrimidine ring; however, for purposes herein, thymine is not considered a methylated nucleotide when present in DNA since thymine is a typical nucleotide base of DNA.

As used herein, a “methylated nucleic acid molecule” refers to a nucleic acid molecule that contains one or more methylated nucleotides.

As used herein, a “methylation state”, “methylation profile”, and “methylation status” of a nucleic acid molecule refers to the presence of absence of one or more methylated nucleotide bases in the nucleic acid molecule. For example, a nucleic acid molecule containing a methylated cytosine is considered methylated (e.g., the methylation state of the nucleic acid molecule is methylated). A nucleic acid molecule that does not contain any methylated nucleotides is considered unmethylated.

The methylation state of a particular nucleic acid sequence (e.g., a gene marker or DNA region as described herein) can indicate the methylation state of every base in the sequence or can indicate the methylation state of a subset of the bases (e.g., of one or more cytosines) within the sequence, or can indicate information regarding regional methylation density within the sequence with or without providing precise information of the locations within the sequence the methylation occurs.

The methylation state of a nucleotide locus in a nucleic acid molecule refers to the presence or absence of a methylated nucleotide at a particular locus in the nucleic acid molecule. For example, the methylation state of a cytosine at the 7th nucleotide in a nucleic acid molecule is methylated when the nucleotide present at the 7th nucleotide in the nucleic acid molecule is 5-methylcytosine. Similarly, the methylation state of a cytosine at the 7th nucleotide in a nucleic acid molecule is unmethylated when the nucleotide present at the 7th nucleotide in the nucleic acid molecule is cytosine (and not 5-methylcytosine).

The methylation status can optionally be represented or indicated by a “methylation value” (e.g., representing a methylation frequency, fraction, ratio, percent, etc.) A methylation value can be generated, for example, by quantifying the amount of intact nucleic acid present following restriction digestion with a methylation dependent restriction enzyme or by comparing amplification profiles after bisulfite reaction or by comparing sequences of bisulfite-treated and untreated nucleic acids. Accordingly, a value, e.g., a methylation value, represents the methylation status and can thus be used as a quantitative indicator of methylation status across multiple copies of a locus. This is of particular use when it is desirable to compare the methylation status of a sequence in a sample to a threshold or reference value.

In some embodiments, the sample is a stool sample, a tissue sample, a blood sample (e.g., plasma sample, whole blood sample, serum sample), or a urine sample. In some embodiments, the sample comprises blood, serum, plasma, gastric secretions, pancreatic juice, a cerebral spinal fluid (CSF) sample, a gastrointestinal biopsy sample, and/or cells recovered from stool. In some embodiments, the subject is human. The sample may include cells, secretions, or tissues from the lymph gland, breast, liver, bile ducts, pancreas, stomach, colon, rectum, esophagus, small intestine, appendix, duodenum, polyps, gall bladder, anus, and/or peritoneum. In some embodiments, the sample comprises cellular fluid, ascites, urine, feces, gastric section, pancreatic fluid, fluid obtained during endoscopy, blood.

As used herein, “methylation frequency” or “methylation percent (%)” refer to the number of instances in which a molecule or locus is methylated relative to the number of instances the molecule or locus is unmethylated.

As such, the methylation state describes the state of methylation of a nucleic acid (e.g., a genomic sequence). In addition, the methylation state refers to the characteristics of a nucleic acid segment at a particular genomic locus relevant to methylation. Such characteristics include, but are not limited to, whether any of the cytosine (C) residues within this DNA sequence are methylated, the location of methylated C residue(s), the frequency or percentage of methylated C throughout any particular region of a nucleic acid, and allelic differences in methylation due to, e.g., difference in the origin of the alleles. The terms “methylation state”, “methylation profile”, and “methylation status” also refer to the relative concentration, absolute concentration, or pattern of methylated C or unmethylated C throughout any particular region of a nucleic acid in a biological sample. For example, if the cytosine (C) residue(s) within a nucleic acid sequence are methylated it may be referred to as “hypermethylated” or having “increased methylation”, whereas if the cytosine (C) residue(s) within a DNA sequence are not methylated it may be referred to as “hypomethylated” or having “decreased methylation”. Likewise, if the cytosine (C) residue(s) within a nucleic acid sequence are methylated as compared to another nucleic acid sequence (e.g., from a different region or from a different individual, etc.) that sequence is considered hypermethylated or having increased methylation compared to the other nucleic acid sequence. Alternatively, if the cytosine (C) residue(s) within a DNA sequence are not methylated as compared to another nucleic acid sequence (e.g., from a different region or from a different individual, etc.) that sequence is considered hypomethylated or having decreased methylation compared to the other nucleic acid sequence. Additionally, the term “methylation pattern” as used herein refers to the collective sites of methylated and unmethylated nucleotides over a region of a nucleic acid. Two nucleic acids may have the same or similar methylation frequency or methylation percent but have different methylation patterns when the number of methylated and unmethylated nucleotides are the same or similar throughout the region but the locations of methylated and unmethylated nucleotides are different. Sequences are said to be “differentially methylated” or as having a “difference in methylation” or having a “different methylation state” when they differ in the extent (e.g., one has increased or decreased methylation relative to the other), frequency, or pattern of methylation. The term “differential methylation” refers to a difference in the level or pattern of nucleic acid methylation in a cancer positive sample as compared with the level or pattern of nucleic acid methylation in a cancer negative sample. It may also refer to the difference in levels or patterns between patients that have recurrence of cancer after surgery versus patients who not have recurrence. Differential methylation and specific levels or patterns of DNA methylation are prognostic and predictive biomarkers, e.g., once the correct cut-off or predictive characteristics have been defined.

Methylation state frequency can be used to describe a population of individuals or a sample from a single individual. For example, a nucleotide locus having a methylation state frequency of 50% is methylated in 50% of instances and unmethylated in 50% of instances. Such a frequency can be used, for example, to describe the degree to which a nucleotide locus or nucleic acid region is methylated in a population of individuals or a collection of nucleic acids. Thus, when methylation in a first population or pool of nucleic acid molecules is different from methylation in a second population or pool of nucleic acid molecules, the methylation state frequency of the first population or pool will be different from the methylation state frequency of the second population or pool. Such a frequency also can be used, for example, to describe the degree to which a nucleotide locus or nucleic acid region is methylated in a single individual. For example, such a frequency can be used to describe the degree to which a group of cells from a tissue sample are methylated or unmethylated at a nucleotide locus or nucleic acid region.

As used herein a “nucleotide locus” refers to the location of a nucleotide in a nucleic acid molecule. A nucleotide locus of a methylated nucleotide refers to the location of a methylated nucleotide in a nucleic acid molecule.

Typically, methylation of human DNA occurs on a dinucleotide sequence including an adjacent guanine and cytosine where the cytosine is located 5′ of the guanine (also termed CpG dinucleotide sequences). Most cytosines within the CpG dinucleotides are methylated in the human genome, however some remain unmethylated in specific CpG dinucleotide rich genomic regions, known as CpG islands (see, e.g, Antequera et al. (1990) Cell 62: 503-514).

As used herein, a “CpG island” refers to a G:C-rich region of genomic DNA containing an increased number of CpG dinucleotides relative to total genomic DNA. A CpG island can be at least 100, 200, or more base pairs in length, where the G:C content of the region is at least 50% and the ratio of observed CpG frequency over expected frequency is 0.6; in some instances, a CpG island can be at least 500 base pairs in length, where the G:C content of the region is at least 55%) and the ratio of observed CpG frequency over expected frequency is 0.65. The observed CpG frequency over expected frequency can be calculated according to the method provided in Gardiner-Garden et al (1987) J. Mol. Biol. 196: 261-281. For example, the observed CpG frequency over expected frequency can be calculated according to the formula R=(A×B)/(C×D), where R is the ratio of observed CpG frequency over expected frequency, A is the number of CpG dinucleotides in an analyzed sequence, B is the total number of nucleotides in the analyzed sequence, C is the total number of C nucleotides in the analyzed sequence, and D is the total number of G nucleotides in the analyzed sequence. Methylation state is typically determined in CpG islands, e.g., at promoter regions. It will be appreciated though that other sequences in the human genome are prone to DNA methylation such as CpA and CpT (see Ramsahoye (2000) Proc. Natl. Acad. Sci. USA 97: 5237-5242; Salmon and Kaye (1970) Biochim. Biophys. Acta. 204: 340-351; Grafstrom (1985) Nucleic Acids Res. 13: 2827-2842; Nyce (1986) Nucleic Acids Res. 14: 4353-4367; Woodcock (1987) Biochem. Biophys. Res. Commun. 145: 888-894).

As used herein, a “methylation-specific reagent” refers to a reagent that modifies a nucleotide of the nucleic acid molecule as a function of the methylation state of the nucleic acid molecule, or a methylation-specific reagent, refers to a compound or composition or other agent that can change the nucleotide sequence of a nucleic acid molecule in a manner that reflects the methylation state of the nucleic acid molecule. Methods of treating a nucleic acid molecule with such a reagent can include contacting the nucleic acid molecule with the reagent, coupled with additional steps, if desired, to accomplish the desired change of nucleotide sequence. Such methods can be applied in a manner in which unmethylated nucleotides (e.g., each unmethylated cytosine) is modified to a different nucleotide. For example, in some embodiments, such a reagent can deaminate unmethylated cytosine nucleotides to produce deoxy uracil residues. Examples of such reagents include, but are not limited to, a methylation-sensitive restriction enzyme, a methylation-dependent restriction enzyme, and a bisulfite reagent.

A change in the nucleic acid nucleotide sequence by a methylation-specific reagent can also result in a nucleic acid molecule in which each methylated nucleotide is modified to a different nucleotide.

As used herein, the term “UDP glucose modified with a chemoselective group” refers to a uridine diphosphoglucose molecule that has been functionalized, particularly at the 6-hydroxyl position, with a functional group capable of reaction with an affinity tag via click chemistry.

The term “oxidized 5-methylcytosine” refers to an oxidized 5-methylcytosine residue that has been oxidized at the 5-position. Oxidized 5-methylcytosine residues thus include 5-hydroxymethylcytosine, 5-formylcytosine, and 5-carboxymethylcytosine. The oxidized 5-methylcytosine residues that undergo reaction with an organic borane according to one embodiment of the invention are 5-formylcytosine and 5-carboxymethylcytosine.

The term “methylation assay” refers to any assay for determining the methylation state of one or more CpG dinucleotide sequences within a sequence of a nucleic acid.

The term “MS AP-PCR” (Methylation-Sensitive Arbitrarily-Primed Polymerase Chain Reaction) refers to the art-recognized technology that allows for a global scan of the genome using CG-rich primers to focus on the regions most likely to contain CpG dinucleotides, and described by Gonzalgo et al. (1997) Cancer Research 57: 594-599.

The term “MethyLight™” refers to the art-recognized fluorescence-based real-time PCR technique described by Eads et al. (1999) Cancer Res. 59: 2302-2306.

The term “HeavyMethyl™” refers to an assay wherein methylation specific blocking probes (also referred to herein as blockers) covering CpG positions between, or covered by, the amplification primers enable methylation-specific selective amplification of a nucleic acid sample.

The term “HeavyMethyl™ MethyLight™” assay refers to a HeavyMethyl™ MethyLight™ assay, which is a variation of the MethyLight™ assay, wherein the MethyLight™ assay is combined with methylation specific blocking probes covering CpG positions between the amplification primers.

The term “Ms-SNuPE” (Methylation-sensitive Single Nucleotide Primer Extension) refers to the art-recognized assay described by Gonzalgo & Jones (1997) Nucleic Acids Res. 25: 2529-2531.

The term “MSP” (Methylation-specific PCR) refers to the art-recognized methylation assay described by Herman et al. (1996) Proc. Natl. Acad. Sci. USA 93: 9821-9826, and by U.S. Pat. No. 5,786,146.

The term “COBRA” (Combined Bisulfite Restriction Analysis) refers to the art-recognized methylation assay described by Xiong & Laird (1997) Nucleic Acids Res. 25: 2532-2534.

The term “MCA” (Methylated CpG Island Amplification) refers to the methylation assay described by Toyota et al. (1999) Cancer Res. 59: 2307-12, and in WO 00/26401A1.

As used herein, a “selected nucleotide” refers to one nucleotide of the four typically occurring nucleotides in a nucleic acid molecule (C, G, T, and A for DNA and C, G, U, and A for RNA), and can include methylated derivatives of the typically occurring nucleotides (e.g., when C is the selected nucleotide, both methylated and unmethylated C are included within the meaning of a selected nucleotide), whereas a methylated selected nucleotide refers specifically to a methylated typically occurring nucleotide and an unmethylated selected nucleotides refers specifically to an unmethylated typically occurring nucleotide.

The term “methylation-specific restriction enzyme” refers to a restriction enzyme that selectively digests a nucleic acid dependent on the methylation state of its recognition site. In the case of a restriction enzyme that specifically cuts if the recognition site is not methylated or is hemi-methylated (a methylation-sensitive enzyme), the cut will not take place (or will take place with a significantly reduced efficiency) if the recognition site is methylated on one or both strands. In the case of a restriction enzyme that specifically cuts only if the recognition site is methylated (a methylation-dependent enzyme), the cut will not take place (or will take place with a significantly reduced efficiency) if the recognition site is not methylated. Preferred are methylation-specific restriction enzymes, the recognition sequence of which contains a CG dinucleotide (for instance a recognition sequence such as CGCG or CCCGGG). Further preferred for some embodiments are restriction enzymes that do not cut if the cytosine in this dinucleotide is methylated at the carbon atom C5.

As used herein, a “different nucleotide” refers to a nucleotide that is chemically different from a selected nucleotide, typically such that the different nucleotide has Watson-Crick base-pairing properties that differ from the selected nucleotide, whereby the typically occurring nucleotide that is complementary to the selected nucleotide is not the same as the typically occurring nucleotide that is complementary to the different nucleotide. For example, when C is the selected nucleotide, U or T can be the different nucleotide, which is exemplified by the complementarity of C to G and the complementarity of U or T to A. As used herein, a nucleotide that is complementary to the selected nucleotide or that is complementary to the different nucleotide refers to a nucleotide that base-pairs, under high stringency conditions, with the selected nucleotide or different nucleotide with higher affinity than the complementary nucleotide's base-paring with three of the four typically occurring nucleotides. An example of complementarity is Watson-Crick base pairing in DNA (e.g., A-T and C-G) and RNA (e.g., A-U and C-G). Thus, for example, G base-pairs, under high stringency conditions, with higher affinity to C than G base-pairs to G, A, or T and, therefore, when C is the selected nucleotide, G is a nucleotide complementary to the selected nucleotide.

As used herein, the “sensitivity” of a given marker (or set of markers used together) refers to the percentage of samples that report a DNA methylation value above a threshold value that distinguishes between neoplastic and non-neoplastic samples. In some embodiments, a positive is defined as a histology-confirmed neoplasia that reports a DNA methylation value above a threshold value (e.g., the range associated with disease), and a false negative is defined as a histology-confirmed neoplasia that reports a DNA methylation value below the threshold value (e.g., the range associated with no disease). The value of sensitivity, therefore, reflects the probability that a DNA methylation measurement for a given marker obtained from a known diseased sample will be in the range of disease-associated measurements. As defined here, the clinical relevance of the calculated sensitivity value represents an estimation of the probability that a given marker would detect the presence of a clinical condition when applied to a subject with that condition.

As used herein, the “specificity” of a given marker (or set of markers used together) refers to the percentage of non-neoplastic samples that report a DNA methylation value below a threshold value that distinguishes between neoplastic and non-neoplastic samples. In some embodiments, a negative is defined as a histology-confirmed non-neoplastic sample that reports a DNA methylation value below the threshold value (e.g., the range associated with no disease) and a false positive is defined as a histology-confirmed non-neoplastic sample that reports a DNA methylation value above the threshold value (e.g., the range associated with disease). The value of specificity, therefore, reflects the probability that a DNA methylation measurement for a given marker obtained from a known non-neoplastic sample will be in the range of non-disease associated measurements. As defined here, the clinical relevance of the calculated specificity value represents an estimation of the probability that a given marker would detect the absence of a clinical condition when applied to a patient without that condition.

The term “AUC” as used herein is an abbreviation for the “area under a curve”. In particular it refers to the area under a Receiver Operating Characteristic (ROC) curve. The ROC curve is a plot of the true positive rate against the false positive rate for the different possible cut points of a diagnostic test. It shows the trade-off between sensitivity and specificity depending on the selected cut point (any increase in sensitivity will be accompanied by a decrease in specificity). The area under an ROC curve (AUC) is a measure for the accuracy of a diagnostic test (the larger the area the better; the optimum is 1; a random test would have a ROC curve lying on the diagonal with an area of 0.5; for reference: J. P. Egan. (1975) Signal Detection Theory and ROC Analysis, Academic Press, New York).

The term “neoplasm” as used herein refers to any new and abnormal growth of tissue. Thus, a neoplasm can be a premalignant neoplasm or a malignant neoplasm.

The term “neoplasm-specific marker,” as used herein, refers to any biological material or element that can be used to indicate the presence of a neoplasm. Examples of biological materials include, without limitation, nucleic acids, polypeptides, carbohydrates, fatty acids, cellular components (e.g., cell membranes and mitochondria), and whole cells. In some instances, markers are particular nucleic acid regions (e.g., genes, intragenic regions, specific loci, etc.). Regions of nucleic acid that are markers may be referred to, e.g., as “marker genes,” “marker regions,” “marker sequences,” “marker loci,” etc.

As used herein, the term “adenoma” refers to a benign tumor of glandular origin. Although these growths are benign, over time they may progress to become malignant.

The term “pre-cancerous” or “pre-neoplastic” and equivalents thereof refer to any cellular proliferative disorder that is undergoing malignant transformation.

A “site” of a neoplasm, adenoma, cancer, etc. is the tissue, organ, cell type, anatomical area, body part, etc. in a subject's body where the neoplasm, adenoma, cancer, etc. is located.

As used herein, a “diagnostic” test application includes the detection or identification of a disease state or condition of a subject, determining the likelihood that a subject will contract a given disease or condition, determining the likelihood that a subject with a disease or condition will respond to therapy, determining the prognosis of a subject with a disease or condition (or its likely progression or regression), and determining the effect of a treatment on a subject with a disease or condition. For example, a diagnostic can be used for detecting the presence or likelihood of a subject contracting a neoplasm or the likelihood that such a subject will respond favorably to a compound (e.g., a pharmaceutical, e.g., a drug) or other treatment.

The term “isolated” when used in relation to a nucleic acid, as in “an isolated oligonucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids, such as DNA and RNA, are found in the state they exist in nature. Examples of non-isolated nucleic acids include: a given DNA sequence (e.g., a gene) found on the host cell chromosome in proximity to neighboring genes; RNA sequences, such as a specific mRNA sequence encoding a specific protein, found in the cell as a mixture with numerous other mRNAs which encode a multitude of proteins. However, isolated nucleic acid encoding a particular protein includes, by way of example, such nucleic acid in cells ordinarily expressing the protein, where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be utilized to express a protein, the oligonucleotide will contain at a minimum the sense or coding strand (i.e., the oligonucleotide may be single-stranded), but may contain both the sense and anti-sense strands (i.e., the oligonucleotide may be double-stranded). An isolated nucleic acid may, after isolation from its natural or typical environment, be combined with other nucleic acids or molecules. For example, an isolated nucleic acid may be present in a host cell in which it has been placed, e.g., for heterologous expression.

The term “purified” refers to molecules, either nucleic acid or amino acid sequences that are removed from their natural environment, isolated, or separated. An “isolated nucleic acid sequence” may therefore be a purified nucleic acid sequence. “Substantially purified” molecules are at least 60% free, preferably at least 75% free, and more preferably at least 90% free from other components with which they are naturally associated. As used herein, the terms “purified” or “to purify” also refer to the removal of contaminants from a sample. The removal of contaminating proteins results in an increase in the percent of polypeptide or nucleic acid of interest in the sample. In another example, recombinant polypeptides are expressed in plant, bacterial, yeast, or mammalian host cells and the polypeptides are purified by the removal of host cell proteins; the percent of recombinant polypeptides is thereby increased in the sample.

The term “composition comprising” a given polynucleotide sequence or polypeptide refers broadly to any composition containing the given polynucleotide sequence or polypeptide. The composition may comprise an aqueous solution containing salts (e.g., NaCl), detergents (e.g., SDS), and other components (e.g., Denhardt's solution, dry milk, salmon sperm DNA, etc.).

The term “sample” is used in its broadest sense. In one sense it can refer to an animal cell or tissue. In another sense, it refers to a specimen or culture obtained from any source, as well as biological and environmental samples. Biological samples may be obtained from plants or animals (including humans) and encompass fluids, solids, tissues, and gases. Environmental samples include environmental material such as surface matter, soil, water, and industrial samples. These examples are not to be construed as limiting the sample types applicable to the present invention.

As used herein, a “remote sample” as used in some contexts relates to a sample collected from a site that is not the cell, tissue, or organ source of the sample.

As used herein, the terms “patient” or “subject” refer to organisms to be subject to various tests provided by the technology. The term “subject” includes animals, preferably mammals, including humans. In a preferred embodiment, the subject is a primate. In an even more preferred embodiment, the subject is a human. Further with respect to diagnostic methods, a preferred subject is a vertebrate subject. A preferred vertebrate is warm-blooded; a preferred warm-blooded vertebrate is a mammal. A preferred mammal is most preferably a human. As used herein, the term “subject’ includes both human and animal subjects. Thus, veterinary therapeutic uses are provided herein. As such, the present technology provides for the diagnosis of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; pinnipeds; and horses. Thus, also provided is the diagnosis and treatment of livestock, including, but not limited to, domesticated swine, ruminants, ungulates, horses (including race horses), and the like. The presently-disclosed subject matter further includes a system for diagnosing a lung cancer in a subject. The system can be provided, for example, as a commercial kit that can be used to screen for a risk of lung cancer or diagnose a lung cancer in a subject from whom a biological sample has been collected. An exemplary system provided in accordance with the present technology includes assessing the methylation state of a marker described herein.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to delivery systems comprising two or more separate containers that each contain a subportion of the total kit components. The containers may be delivered to the intended recipient together or separately. For example, a first container may contain an enzyme for use in an assay, while a second container contains oligonucleotides. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

As used herein, the term “information” refers to any collection of facts or data. In reference to information stored or processed using a computer system(s), including but not limited to internets, the term refers to any data stored in any format (e.g., analog, digital, optical, etc.). As used herein, the term “information related to a subject” refers to facts or data pertaining to a subject (e.g., a human, plant, or animal). The term “genomic information” refers to information pertaining to a genome including, but not limited to, nucleic acid sequences, genes, percentage methylation, allele frequencies, RNA expression levels, protein expression, phenotypes correlating to genotypes, etc. “Allele frequency information” refers to facts or data pertaining to allele frequencies, including, but not limited to, allele identities, statistical correlations between the presence of an allele and a characteristic of a subject (e.g., a human subject), the presence or absence of an allele in an individual or population, the percentage likelihood of an allele being present in an individual having one or more particular characteristics, etc.

### DETAILED DESCRIPTION

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

Provided herein is technology for PNET screening and particularly, but not exclusively, to methods, compositions, and related uses for detecting the presence of PNET and/or related NET types (e.g., lung NET, small bowel NET). As the technology is described herein, the section headings used are for organizational purposes only and are not to be construed as limiting the subject matter in any way.

Indeed, as described in Example I, experiments conducted during the course for identifying embodiments for the present invention identified a novel set of 198 differentially methylated regions (DMRs) for discriminating PNET derived DNA from non-neoplastic control DNA. From these 198 novel DNA methylation markers, further experiments identified markers capable of distinguishing PNET from normal pancreatic tissue and detecting PNET in blood.

Although the disclosure herein refers to certain illustrated embodiments, it is to be understood that these embodiments are presented by way of example and not by way of limitation.

In particular aspects, the present technology provides compositions and methods for identifying, determining, and/or classifying a cancer such as PNET. The methods comprise determining the methylation status of at least one methylation marker in a biological sample isolated from a subject (e.g., stool sample, pancreatic tissue sample, plasma sample), wherein a change in the methylation state of the marker is indicative of the presence, class, or site of PNET. Particular embodiments relate to markers comprising a differentially methylated region (DMR, e.g., DMR 1-198, see Tables TA and 2A) that are used for diagnosis (e.g., screening) of PNET and various NET types (e.g., lung NET, small bowel NET).

In addition to embodiments wherein the methylation analysis of at least one marker, a region of a marker, or a base of a marker comprising a DMR (e.g., DMR, e.g., DMR 1-198) provided herein and listed in Tables 1A and 2A is analyzed, the technology also provides panels of markers comprising at least one marker, region of a marker, or base of a marker comprising a DMR with utility for the detection of cancers, in particular PNET.

Some embodiments of the technology are based upon the analysis of the CpG methylation status of at least one marker, region of a marker, or base of a marker comprising a DMR.

In some embodiments, the present technology provides for the use of a reagent that modifies DNA in a methylation-specific manner (e.g., a methylation-sensitive restriction enzyme, a methylation-dependent restriction enzyme, and a bisulfite reagent) in combination with one or more methylation assays to determine the methylation status of CpG dinucleotide sequences within at least one marker comprising a DMR (e.g., DMR 1-198, see Tables 1A and 2A). Genomic CpG dinucleotides can be methylated or unmethylated (alternatively known as up- and down-methylated respectively). However the methods of the present invention are suitable for the analysis of biological samples of a heterogeneous nature, e.g., a low concentration of tumor cells, or biological materials therefrom, within a background of a remote sample (e.g., blood, organ effluent, or stool). Accordingly, when analyzing the methylation status of a CpG position within such a sample one may use a quantitative assay for determining the level (e.g., percent, fraction, ratio, proportion, or degree) of methylation at a particular CpG position.

According to the present technology, determination of the methylation status of CpG dinucleotide sequences in markers comprising a DMR has utility both in the diagnosis and characterization of cancers such as PNET.

### Combinations of Markers

A frequently used method for analyzing a nucleic acid for the presence of 5-methylcytosine is based upon the bisulfite method described by Frommer, et al. for the detection of 5-methylcytosines in DNA (Frommer et al. (1992) Proc. Natl. Acad. Sci. USA 89: 1827-31 explicitly incorporated herein by reference in its entirety for all purposes) or variations thereof. The bisulfite method of mapping 5-methylcytosines is based on the observation that cytosine, but not 5-methylcytosine, reacts with hydrogen sulfite ion (also known as bisulfite). The reaction is usually performed according to the following steps: first, cytosine reacts with hydrogen sulfite to form a sulfonated cytosine. Next, spontaneous deamination of the sulfonated reaction intermediate results in a sulfonated uracil. Finally, the sulfonated uracil is desulfonated under alkaline conditions to form uracil. Detection is possible because uracil base pairs with adenine (thus behaving like thymine), whereas 5-methylcytosine base pairs with guanine (thus behaving like cytosine). This makes the discrimination of methylated cytosines from non-methylated cytosines possible by, e.g., bisulfite genomic sequencing (Grigg G, & Clark S, Bioessays (1994) 16: 431-36; Grigg G, DNA Seq. (1996) 6: 189-98), methylation-specific PCR (MSP) as is disclosed, e.g., in U.S. Pat. No. 5,786,146, or using an assay comprising sequence-specific probe cleavage, e.g., a QuARTS flap endonuclease assay (see, e.g., Zou et al. (2010) “Sensitive quantification of methylated markers with a novel methylation specific technology” Clin Chem 56: A199; and in U.S. Pat. Nos. 8,361,720; 8,715,937; 8,916,344; and 9,212,392).

Some conventional technologies are related to methods comprising enclosing the DNA to be analyzed in an agarose matrix, thereby preventing the diffusion and renaturation of the DNA (bisulfite only reacts with single-stranded DNA), and replacing precipitation and purification steps with a fast dialysis (Olek A, et al. (1996) “A modified and improved method for bisulfite based cytosine methylation analysis” Nucleic Acids Res. 24: 5064-6). It is thus possible to analyze individual cells for methylation status, illustrating the utility and sensitivity of the method. An overview of conventional methods for detecting 5-methylcytosine is provided by Rein, T., et al. (1998) Nucleic Acids Res. 26: 2255.

The bisulfite technique typically involves amplifying short, specific fragments of a known nucleic acid subsequent to a bisulfite treatment, then either assaying the product by sequencing (Olek & Walter (1997) Nat. Genet. 17: 275-6) or a primer extension reaction (Gonzalgo & Jones (1997) Nucleic Acids Res. 25: 2529-31; WO 95/00669; U.S. Pat. No. 6,251,594) to analyze individual cytosine positions. Some methods use enzymatic digestion (Xiong & Laird (1997) Nucleic Acids Res. 25: 2532-4). Detection by hybridization has also been described in the art (Olek et al., WO 99/28498). Additionally, use of the bisulfite technique for methylation detection with respect to individual genes has been described (Grigg & Clark (1994) Bioessays 16: 431-6; Zeschnigk et al. (1997) Hum Mol Genet. 6: 387-95; Feil et al. (1994) Nucleic Acids Res. 22: 695; Martin et al. (1995) Gene 157: 261-4; WO 9746705; WO 9515373).

Various methylation assay procedures can be used in conjunction with bisulfite treatment according to the present technology. These assays allow for determination of the methylation state of one or a plurality of CpG dinucleotides (e.g., CpG islands) within a nucleic acid sequence. Such assays involve, among other techniques, sequencing of bisulfite-treated nucleic acid, PCR (for sequence-specific amplification), Southern blot analysis, and use of methylation-specific restriction enzymes, e.g., methylation-sensitive or methylation-dependent enzymes.

For example, genomic sequencing has been simplified for analysis of methylation patterns and 5-methylcytosine distributions by using bisulfite treatment (Frommer et al. (1992) Proc. Natl. Acad. Sci. USA 89: 1827-1831). Additionally, restriction enzyme digestion of PCR products amplified from bisulfite-converted DNA finds use in assessing methylation state, e.g., as described by Sadri & Hornsby (1997) Nucl. Acids Res. 24: 5058-5059 or as embodied in the method known as COBRA (Combined Bisulfite Restriction Analysis) (Xiong & Laird (1997) Nucleic Acids Res. 25: 2532-2534).

COBRA™ analysis is a quantitative methylation assay useful for determining DNA methylation levels at specific loci in small amounts of genomic DNA (Xiong & Laird, Nucleic Acids Res. 25:2532-2534, 1997). Briefly, restriction enzyme digestion is used to reveal methylation-dependent sequence differences in PCR products of sodium bisulfite-treated DNA. Methylation-dependent sequence differences are first introduced into the genomic DNA by standard bisulfite treatment according to the procedure described by Frommer et al. (Proc. Natl. Acad. Sci. USA 89:1827-1831, 1992). PCR amplification of the bisulfite converted DNA is then performed using primers specific for the CpG islands of interest, followed by restriction endonuclease digestion, gel electrophoresis, and detection using specific, labeled hybridization probes. Methylation levels in the original DNA sample are represented by the relative amounts of digested and undigested PCR product in a linearly quantitative fashion across a wide spectrum of DNA methylation levels. In addition, this technique can be reliably applied to DNA obtained from microdissected paraffin-embedded tissue samples.

Typical reagents (e.g., as might be found in a typical COBRA™-based kit) for COBRA™ analysis may include, but are not limited to: PCR primers for specific loci (e.g., specific genes, markers, DMR, regions of genes, regions of markers, bisulfite treated DNA sequence, CpG island, etc.); restriction enzyme and appropriate buffer; gene-hybridization oligonucleotide; control hybridization oligonucleotide; kinase labeling kit for oligonucleotide probe; and labeled nucleotides. Additionally, bisulfite conversion reagents may include: DNA denaturation buffer; sulfonation buffer; DNA recovery reagents or kits (e.g., precipitation, ultrafiltration, affinity column); desulfonation buffer; and DNA recovery components. Assays such as “MethyLight™” (a fluorescence-based real-time PCR technique) (Eads et al., Cancer Res. 59:2302-2306, 1999), Ms-SNuPE™ (Methylation-sensitive Single Nucleotide Primer Extension) reactions (Gonzalgo & Jones, Nucleic Acids Res. 25:2529-2531, 1997), methylation-specific PCR (“MSP”; Herman et al., Proc. Natl. Acad. Sci. USA 93:9821-9826, 1996; U.S. Pat. No. 5,786,146), and methylated CpG island amplification (“MCA”; Toyota et al., Cancer Res. 59:2307-12, 1999) are used alone or in combination with one or more of these methods.

The “HeavyMethyl™” assay, technique is a quantitative method for assessing methylation differences based on methylation-specific amplification of bisulfite-treated DNA. Methylation-specific blocking probes (“blockers”) covering CpG positions between, or covered by, the amplification primers enable methylation-specific selective amplification of a nucleic acid sample.

The term “HeavyMethyl™ MethyLight™” assay refers to a HeavyMethyl™ MethyLight™ assay, which is a variation of the MethyLight™ assay, wherein the MethyLight™ assay is combined with methylation specific blocking probes covering CpG positions between the amplification primers. The HeavyMethyl™ assay may also be used in combination with methylation specific amplification primers.

Typical reagents (e.g., as might be found in a typical MethyLight™-based kit) for HeavyMethyl™ analysis may include, but are not limited to: PCR primers for specific loci (e.g., specific genes, markers, regions of genes, regions of markers, bisulfite treated DNA sequence, CpG island, or bisulfite treated DNA sequence or CpG island, etc.); blocking oligonucleotides; optimized PCR buffers and deoxynucleotides; and Taq polymerase. MSP (methylation-specific PCR) allows for assessing the methylation status of virtually any group of CpG sites within a CpG island, independent of the use of methylation-sensitive restriction enzymes (Herman et al. Proc. Natl. Acad. Sci. USA 93:9821-9826, 1996; U.S. Pat. No. 5,786,146). Briefly, DNA is modified by sodium bisulfite, which converts unmethylated, but not methylated cytosines, to uracil, and the products are subsequently amplified with primers specific for methylated versus unmethylated DNA. MSP requires only small quantities of DNA, is sensitive to 0.1% methylated alleles of a given CpG island locus, and can be performed on DNA extracted from paraffin-embedded samples. Typical reagents (e.g., as might be found in a typical MSP-based kit) for MSP analysis may include, but are not limited to: methylated and unmethylated PCR primers for specific loci (e.g., specific genes, markers, regions of genes, regions of markers, bisulfite treated DNA sequence, CpG island, etc.); optimized PCR buffers and deoxynucleotides, and specific probes.

The MethyLight™ assay is a high-throughput quantitative methylation assay that utilizes fluorescence-based real-time PCR (e.g., TaqMan®) that requires no further manipulations after the PCR step (Eads et al., Cancer Res. 59:2302-2306, 1999). Briefly, the MethyLight™ process begins with a mixed sample of genomic DNA that is converted, in a sodium bisulfite reaction, to a mixed pool of methylation-dependent sequence differences according to standard procedures (the bisulfite process converts unmethylated cytosine residues to uracil). Fluorescence-based PCR is then performed in a “biased” reaction, e.g., with PCR primers that overlap known CpG dinucleotides. Sequence discrimination occurs both at the level of the amplification process and at the level of the fluorescence detection process.

The MethyLight™ assay is used as a quantitative test for methylation patterns in a nucleic acid, e.g., a genomic DNA sample, wherein sequence discrimination occurs at the level of probe hybridization. In a quantitative version, the PCR reaction provides for a methylation specific amplification in the presence of a fluorescent probe that overlaps a particular putative methylation site. An unbiased control for the amount of input DNA is provided by a reaction in which neither the primers, nor the probe, overlie any CpG dinucleotides. Alternatively, a qualitative test for genomic methylation is achieved by probing the biased PCR pool with either control oligonucleotides that do not cover known methylation sites (e.g., a fluorescence-based version of the HeavyMethyl™ and MSP techniques) or with oligonucleotides covering potential methylation sites.

The MethyLight™ process is used with any suitable probe (e.g. a “TaqMan®” probe, a Lightcycler® probe, etc.) For example, in some applications double-stranded genomic DNA is treated with sodium bisulfite and subjected to one of two sets of PCR reactions using TaqMan® probes, e.g., with MSP primers and/or HeavyMethyl blocker oligonucleotides and a TaqMan® probe. The TaqMan® probe is dual-labeled with fluorescent “reporter” and “quencher” molecules and is designed to be specific for a relatively high GC content region so that it melts at about a 10° C. higher temperature in the PCR cycle than the forward or reverse primers. This allows the TaqMan® probe to remain fully hybridized during the PCR annealing/extension step. As the Taq polymerase enzymatically synthesizes a new strand during PCR, it will eventually reach the annealed TaqMan® probe. The Taq polymerase 5′ to 3′ endonuclease activity will then displace the TaqMan® probe by digesting it to release the fluorescent reporter molecule for quantitative detection of its now unquenched signal using a real-time fluorescent detection system.

Typical reagents (e.g., as might be found in a typical MethyLight™-based kit) for MethyLight™ analysis may include, but are not limited to: PCR primers for specific loci (e.g., specific genes, markers, regions of genes, regions of markers, bisulfite treated DNA sequence, CpG island, etc.); TaqMan® or Lightcycler® probes; optimized PCR buffers and deoxynucleotides; and Taq polymerase.

The QM™ (quantitative methylation) assay is an alternative quantitative test for methylation patterns in genomic DNA samples, wherein sequence discrimination occurs at the level of probe hybridization. In this quantitative version, the PCR reaction provides for unbiased amplification in the presence of a fluorescent probe that overlaps a particular putative methylation site. An unbiased control for the amount of input DNA is provided by a reaction in which neither the primers, nor the probe, overlie any CpG dinucleotides. Alternatively, a qualitative test for genomic methylation is achieved by probing the biased PCR pool with either control oligonucleotides that do not cover known methylation sites (a fluorescence-based version of the HeavyMethyl™ and MSP techniques) or with oligonucleotides covering potential methylation sites.

The QM™ process can be used with any suitable probe, e.g., “TaqMan®” probes, Lightcycler® probes, in the amplification process. For example, double-stranded genomic DNA is treated with sodium bisulfite and subjected to unbiased primers and the TaqMan® probe. The TaqMan® probe is dual-labeled with fluorescent “reporter” and “quencher” molecules, and is designed to be specific for a relatively high GC content region so that it melts out at about a 10° C. higher temperature in the PCR cycle than the forward or reverse primers. This allows the TaqMan® probe to remain fully hybridized during the PCR annealing/extension step. As the Taq polymerase enzymatically synthesizes a new strand during PCR, it will eventually reach the annealed TaqMan® probe. The Taq polymerase 5′ to 3′ endonuclease activity will then displace the TaqMan® probe by digesting it to release the fluorescent reporter molecule for quantitative detection of its now unquenched signal using a real-time fluorescent detection system. Typical reagents (e.g., as might be found in a typical QM™-based kit) for QM™ analysis may include, but are not limited to: PCR primers for specific loci (e.g., specific genes, markers, regions of genes, regions of markers, bisulfite treated DNA sequence, CpG island, etc.); TaqMan® or Lightcycler® probes; optimized PCR buffers and deoxynucleotides; and Taq polymerase.

The Ms-SNuPE™ technique is a quantitative method for assessing methylation differences at specific CpG sites based on bisulfite treatment of DNA, followed by single-nucleotide primer extension (Gonzalgo & Jones, Nucleic Acids Res. 25:2529-2531, 1997). Briefly, genomic DNA is reacted with sodium bisulfite to convert unmethylated cytosine to uracil while leaving 5-methylcytosine unchanged. Amplification of the desired target sequence is then performed using PCR primers specific for bisulfite-converted DNA, and the resulting product is isolated and used as a template for methylation analysis at the CpG site of interest. Small amounts of DNA can be analyzed (e.g., microdissected pathology sections) and it avoids utilization of restriction enzymes for determining the methylation status at CpG sites.

Typical reagents (e.g., as might be found in a typical Ms-SNuPE™-based kit) for Ms-SNuPE™ analysis may include, but are not limited to: PCR primers for specific loci (e.g., specific genes, markers, regions of genes, regions of markers, bisulfite treated DNA sequence, CpG island, etc.); optimized PCR buffers and deoxynucleotides; gel extraction kit; positive control primers; Ms-SNuPE™ primers for specific loci; reaction buffer (for the Ms-SNuPE reaction); and labeled nucleotides. Additionally, bisulfite conversion reagents may include: DNA denaturation buffer; sulfonation buffer; DNA recovery reagents or kit (e.g., precipitation, ultrafiltration, affinity column); desulfonation buffer; and DNA recovery components.

Reduced Representation Bisulfite Sequencing (RRBS) begins with bisulfite treatment of nucleic acid to convert all unmethylated cytosines to uracil, followed by restriction enzyme digestion (e.g., by an enzyme that recognizes a site including a CG sequence such as MspI) and complete sequencing of fragments after coupling to an adapter ligand. The choice of restriction enzyme enriches the fragments for CpG dense regions, reducing the number of redundant sequences that may map to multiple gene positions during analysis. As such, RRBS reduces the complexity of the nucleic acid sample by selecting a subset (e.g., by size selection using preparative gel electrophoresis) of restriction fragments for sequencing. As opposed to whole-genome bisulfite sequencing, every fragment produced by the restriction enzyme digestion contains DNA methylation information for at least one CpG dinucleotide. As such, RRBS enriches the sample for promoters, CpG islands, and other genomic features with a high frequency of restriction enzyme cut sites in these regions and thus provides an assay to assess the methylation state of one or more genomic loci.

A typical protocol for RRBS comprises the steps of digesting a nucleic acid sample with a restriction enzyme such as MspI, filling in overhangs and A-tailing, ligating adaptors, bisulfite conversion, and PCR. See, e.g., et al. (2005) “Genome-scale DNA methylation mapping of clinical samples at single-nucleotide resolution” Nat Methods 7: 133-6; Meissner et al. (2005) “Reduced representation bisulfite sequencing for comparative high-resolution DNA methylation analysis” Nucleic Acids Res. 33: 5868-77.

In some embodiments, a quantitative allele-specific real-time target and signal amplification (QuARTS) assay is used to evaluate methylation state. Three reactions sequentially occur in each QuARTS assay, including amplification (reaction 1) and target probe cleavage (reaction 2) in the primary reaction; and FRET cleavage and fluorescent signal generation (reaction 3) in the secondary reaction. When target nucleic acid is amplified with specific primers, a specific detection probe with a flap sequence loosely binds to the amplicon. The presence of the specific invasive oligonucleotide at the target binding site causes a 5′ nuclease, e.g., a FEN-1 endonuclease, to release the flap sequence by cutting between the detection probe and the flap sequence. The flap sequence is complementary to a non-hairpin portion of a corresponding FRET cassette. Accordingly, the flap sequence functions as an invasive oligonucleotide on the FRET cassette and effects a cleavage between the FRET cassette fluorophore and a quencher, which produces a fluorescent signal. The cleavage reaction can cut multiple probes per target and thus release multiple fluorophores per flap, providing exponential signal amplification. QuARTS can detect multiple targets in a single reaction well by using FRET cassettes with different dyes. See, e.g., in Zou et al. (2010) “Sensitive quantification of methylated markers with a novel methylation specific technology” Clin Chem 56: A199), and U.S. Pat. Nos. 8,361,720; 8,715,937; 8,916,344; and 9,212,392, each of which is incorporated herein by reference for all purposes.

The term “bisulfite reagent” refers to a reagent comprising bisulfite, disulfite, hydrogen sulfite, or combinations thereof, useful as disclosed herein to distinguish between methylated and unmethylated CpG dinucleotide sequences. Methods of said treatment are known in the art (e.g., PCT/EP2004/011715 and WO 2013/116375, each of which is incorporated by reference in its entirety). In some embodiments, bisulfite treatment is conducted in the presence of denaturing solvents such as but not limited to n-alkyleneglycol or diethylene glycol dimethyl ether (DME), or in the presence of dioxane or dioxane derivatives. In some embodiments the denaturing solvents are used in concentrations between 1% and 35% (v/v). In some embodiments, the bisulfite reaction is carried out in the presence of scavengers such as but not limited to chromane derivatives, e.g., 6-hydroxy-2,5,7,8,-tetramethylchromane 2-carboxylic acid or trihydroxybenzone acid and derivates thereof, e.g., Gallic acid (see: PCT/EP2004/011715, which is incorporated by reference in its entirety). In certain preferred embodiments, the bisulfite reaction comprises treatment with ammonium hydrogen sulfite, e.g., as described in WO 2013/116375.

In some embodiments, fragments of the treated DNA are amplified using sets of primer oligonucleotides according to the present invention (e.g., see Table V) and an amplification enzyme. The amplification of several DNA segments can be carried out simultaneously in one and the same reaction vessel. Typically, the amplification is carried out using a polymerase chain reaction (PCR). Amplicons are typically 100 to 2000 base pairs in length.

In some embodiments, the technology relates to assessing the methylation state of combinations of markers comprising a DMR from Tables 1A and 2A (e.g., DMR Nos. 1-198). In some embodiments, assessing the methylation state of more than one marker increases the specificity and/or sensitivity of a screen or diagnostic for identifying a neoplasm in a subject (e.g., PNETs as described herein).

In another embodiment, the invention provides a method for converting an oxidized 5-methylcytosine residue in cell-free DNA to a dihydrouracil residue (see, Liu et al., 2019, Nat Biotechnol. 37, pp. 424-429; U.S. Patent Application Publication No. 202000370114). The method involves reaction of an oxidized 5mC residue selected from 5-formylcytosine (5fC), 5-carboxymethylcytosine (5caC), and combinations thereof, with an organic borane. The oxidized 5mC residue may be naturally occurring or, more typically, the result of a prior oxidation of a 5mC or 5hmC residue, e.g., oxidation of 5mC or 5hmC with a TET family enzyme (e.g., TET1, TET2, or TET3), or chemical oxidation of 5 mC or 5hmC, e.g., with potassium perruthenate (KRuO₄) or an inorganic peroxo compound or composition such as peroxotungstate (see, e.g., Okamoto et al. (2011) Chem. Commun. 47:11231-33) and a copper (II) perchlorate/2,2,6,6-tetramethylpiperidine-1-oxyl (TEMPO) combination (see Matsushita et al. (2017) Chem. Commun. 53:5756-59).

The organic borane may be characterized as a complex of borane and a nitrogen-containing compound selected from nitrogen heterocycles and tertiary amines. The nitrogen heterocycle may be monocyclic, bicyclic, or polycyclic, but is typically monocyclic, in the form of a 5- or 6-membered ring that contains a nitrogen heteroatom and optionally one or more additional heteroatoms selected from N, O, and S. The nitrogen heterocycle may be aromatic or alicyclic. Preferred nitrogen heterocycles herein include 2-pyrroline, 2H-pyrrole, 1H-pyrrole, pyrazolidine, imidazolidine, 2-pyrazoline, 2-imidazoline, pyrazole, imidazole, 1,2,4-triazole, 1,2,4-triazole, pyridazine, pyrimidine, pyrazine, 1,2,4-triazine, and 1,3,5-triazine, any of which may be unsubstituted or substituted with one or more non-hydrogen substituents. Typical non-hydrogen substituents are alkyl groups, particularly lower alkyl groups, such as methyl, ethyl, n-propyl, isopropyl, n-butyl, isobutyl, t-butyl, and the like. Exemplary compounds include pyridine borane, 2-methylpyridine borane (also referred to as 2-picoline borane), and 5-ethyl-2-pyridine.

The reaction of the organic borane with the oxidized 5mC residue in cell-free DNA is advantageous insofar as non-toxic reagents and mild reaction conditions can be employed; there is no need for any bisulfite, nor for any other potentially DNA-degrading reagents. Furthermore, conversion of an oxidized 5mC residue to dihydrouracil with the organic borane can be carried out without need for isolation of any intermediates, in a “one-pot” or “one-tube” reaction. This is quite significant, since the conversion involves multiple steps, i.e., (1) reduction of the alkene bond linking C-4 and C-5 in the oxidized 5mC, (2) deamination, and (3) either decarboxylation, if the oxidized 5mC is 5caC, or deformylation, if the oxidized 5mC is 5fC.

In addition to a method for converting an oxidized 5-methylcytosine residue in cell-free DNA to a dihydrouracil residue, the invention also provides a reaction mixture related to the aforementioned method. The reaction mixture comprises a sample of cell-free DNA containing at least one oxidized 5-methylcytosine residue selected from 5caC, 5fC, and combinations thereof, and an organic borane effective to reduce, deaminate, and either decarboxylate or deformylate the at least one oxidized 5-methylcytosine residue. The organic borane is a complex of borane and a nitrogen-containing compound selected from nitrogen heterocycles and tertiary amines, as explained above. In a preferred embodiment, the reaction mixture is substantially free of bisulfite, meaning substantially free of bisulfite ion and bisulfite salts. Ideally, the reaction mixture contains no bisulfite.

In a related aspect of the invention, a kit is provided for converting 5mC residues in cell-free DNA to dihydrouracil residues, where the kit includes a reagent for blocking 5hmC residues, a reagent for oxidizing 5mC residues beyond hydroxymethylation to provide oxidized 5mC residues, and an organic borane effective to reduce, deaminate, and either decarboxylate or deformylate the oxidized 5mC residues. The kit may also include instructions for using the components to carry out the above-described method.

In another embodiment, a method is provided that makes use of the above-described oxidation reaction. The method enables detecting the presence and location of 5-methylcytosine residues in cell-free DNA, and comprises the following steps:

(a) modifying 5hmC residues in fragmented, adapter-ligated cell-free DNA to provide an affinity tag thereon, wherein the affinity tag enables removal of modified 5hmC-containing DNA from the cell-free DNA;

(b) removing the modified 5hmC-containing DNA from the cell-free DNA, leaving DNA containing unmodified 5mC residues;

(c) oxidizing the unmodified 5mC residues to give DNA containing oxidized 5mC residues selected from 5caC, 5fC, and combinations thereof;

(d) contacting the DNA containing oxidized 5mC residues with an organic borane effective to reduce, deaminate, and either decarboxylate or deformylate the oxidized 5mC residues, thereby providing DNA containing dihydrouracil residues in place of the oxidized 5mC residues;

(e) amplifying and sequencing the DNA containing dihydrouracil residues;

(f) determining a 5-methylation pattern from the sequencing results in (e).

The cell-free DNA is extracted from a body sample from a subject, where the body sample is typically whole blood, plasma, or serum, most typically plasma, but the sample may also be urine, saliva, mucosal excretions, sputum, stool, or tears. In some embodiments, the cell-free DNA is derived from a tumor. In other embodiments, the cell-free DNA is from a patient with a disease or other pathogenic condition. The cell-free DNA may or may not derive from a tumor. In step (a), it should be noted that the cell-free DNA in which 5hmC residues are to be modified is in purified, fragmented form, and adapter-ligated. DNA purification in this context can be carried out using any suitable method known to those of ordinary skill in the art and/or described in the pertinent literature, and, while cell-free DNA can itself be highly fragmented, further fragmentation may occasionally be desirable, as described, for example, in U.S. Patent Publication No. 2017/0253924. The cell-free DNA fragments are generally in the size range of about 20 nucleotides to about 500 nucleotides, more typically in the range of about 20 nucleotides to about 250 nucleotides. The purified cell-free DNA fragments that are modified in step (a) have been end-repaired using conventional means (e.g., a restriction enzyme) so that the fragments have a blunt end at each 3′ and 5′ terminus. In a preferred method, as described in WO 2017/176630, the blunted fragments have also been provided with a 3′ overhang comprising a single adenine residue using a polymerase such as Taq polymerase. This facilitates subsequent ligation of a selected universal adapter, i.e., an adapter such as a Y-adapter or a hairpin adapter that ligates to both ends of the cell-free DNA fragments and contains at least one molecular barcode. Use of adapters also enables selective PCR enrichment of adapter-ligated DNA fragments.

In step (a), then, the “purified, fragmented cell-free DNA” comprises adapter-ligated DNA fragments. Modification of 5hmC residues in these cell-free DNA fragments with an affinity tag, as specified in step (a), is done so as to enable subsequent removal of the modified 5hmC-containing DNA from the cell-free DNA. In one embodiment, the affinity tag comprises a biotin moiety, such as biotin, desthiobiotin, oxybiotin, 2-iminobiotin, diaminobiotin, biotin sulfoxide, biocytin, or the like. Use of a biotin moiety as the affinity tag allows for facile removal with streptavidin, e.g., streptavidin beads, magnetic streptavidin beads, etc.

Tagging 5hmC residues with a biotin moiety or other affinity tag is accomplished by covalent attachment of a chemoselective group to 5hmC residues in the DNA fragments, where the chemoselective group is capable of undergoing reaction with a functionalized affinity tag so as to link the affinity tag to the 5hmC residues. In one embodiment, the chemoselective group is UDP glucose-6-azide, which undergoes a spontaneous 1,3-cycloaddition reaction with an alkyne-functionalized biotin moiety, as described in Robertson et al. (2011) Biochem. Biophys. Res. Comm. 411(1):40-3, U.S. Pat. No. 8,741,567, and WO 2017/176630. Addition of an alkyne-functionalized biotin-moiety thus results in covalent attachment of the biotin moiety to each 5hmC residue.

The affinity-tagged DNA fragments can then be pulled down in step (b) using, in one embodiment, streptavidin, in the form of streptavidin beads, magnetic streptavidin beads, or the like, and set aside for later analysis, if so desired. The supernatant remaining after removal of the affinity-tagged fragments contains DNA with unmodified 5mC residues and no 5hmC residues.

In step (c), the unmodified 5mC residues are oxidized to provide 5caC residues and/or 5fC residues, using any suitable means. The oxidizing agent is selected to oxidize 5mC residues beyond hydroxymethylation, i.e., to provide 5caC and/or 5fC residues. Oxidation may be carried out enzymatically, using a catalytically active TET family enzyme. A “TET family enzyme” or a “TET enzyme” as those terms are used herein refer to a catalytically active “TET family protein” or a “TET catalytically active fragment” as defined in U.S. Pat. No. 9,115,386, the disclosure of which is incorporated by reference herein. A preferred TET enzyme in this context is TET2; see Ito et al. (2011) Science 333(6047):1300-1303. Oxidation may also be carried out chemically, as described in the preceding section, using a chemical oxidizing agent. Examples of suitable oxidizing agent include, without limitation: a perruthenate anion in the form of an inorganic or organic perruthenate salt, including metal perruthenates such as potassium perruthenate (KRuO₄), tetraalkylammonium perruthenates such as tetrapropylammonium perruthenate (TPAP) and tetrabutylammonium perruthenate (TBAP), and polymer supported perruthenate (PSP); and inorganic peroxo compounds and compositions such as peroxotungstate or a copper (II) perchlorate/TEMPO combination. It is unnecessary at this point to separate 5fC-containing fragments from 5caC-containing fragments, insofar as in the next step of the process, step (e) converts both 5fC residues and 5caC residues to dihydrouracil (DHU).

In some embodiments, 5-hydroxymethylcytosine residues are blocked with β-glucosyltransferase (β3GT), while 5-methylcytosine residues are oxidized with a TET enzyme effective to provide a mixture of 5-formylcytosine and 5-carboxymethylcytosine. The mixture containing both of these oxidized species can be reacted with 2-picoline borane or another organic borane to give dihydrouracil. In a variation on this embodiment, 5hmC-containing fragments are not removed in step (b). Rather, “TET-Assisted Picoline Borane Sequencing (TAPS),” 5mC-containing fragments and 5hmC-containing fragments are together enzymatically oxidized to provide 5fC- and 5caC-containing fragments. Reaction with 2-picoline borane results in DHU residues wherever 5mC and 5hmC residues were originally present. “Chemical Assisted Picoline Borane Sequencing (CAPS),” involves selective oxidation of 5hmC-containing fragments with potassium perruthenate, leaving 5mC residues unchanged.

There are numerous advantages to the method of this embodiment: bisulfite is unnecessary, nontoxic reagents and reactants are employed; and the process proceeds under mild conditions. In addition, the entire process can be performed in a single tube, without need for isolation of any intermediates.

In a related embodiment, the above method includes a further step: (g) identifying a hydroxymethylation pattern in the 5hmC-containing DNA removed from the cell-free DNA in step (b). This can be carried out using the techniques described in detail in WO 2017/176630. The process can be carried out without removal or isolation of intermediates in a one-tube method. For example, initially, cell-free DNA fragments, preferably adapter-ligated DNA fragments, are subjected to functionalization with OGT-catalyzed uridine diphosphoglucose 6-azide, followed by biotinylation via the chemoselective azide groups. This procedure results in covalently attached biotin at each 5hmC site. In a next step, the biotinylated strands and strands containing unmodified (native) 5mC are pulled down simultaneously for further processing. The native 5mC-containing strands are pulled down using an anti-5mC antibody or a methyl-CpG-binding domain (MBD) protein, as is known in the art. Then, with the 5hmC residues blocked, the unmodified 5mC residues are selectively oxidized using any suitable technique for converting 5mC to 5fC and/or 5caC, as described elsewhere herein.

The fragments obtained by means of the amplification can carry a directly or indirectly detectable label. In some embodiments, the labels are fluorescent labels, radionuclides, or detachable molecule fragments having a typical mass that can be detected in a mass spectrometer. Where said labels are mass labels, some embodiments provide that the labeled amplicons have a single positive or negative net charge, allowing for better delectability in the mass spectrometer. The detection may be carried out and visualized by means of, e.g., matrix assisted laser desorption/ionization mass spectrometry (MALDI) or using electron spray mass spectrometry (ESI).

Methods for isolating DNA suitable for these assay technologies are known in the art. In particular, some embodiments comprise isolation of nucleic acids as described in U.S. patent application Ser. No. 13/470,251 (“Isolation of Nucleic Acids”), incorporated herein by reference in its entirety.

In some embodiments, the markers described herein find use in QUARTS assays performed on stool samples. In some embodiments, methods for producing DNA samples and, in particular, to methods for producing DNA samples that comprise highly purified, low-abundance nucleic acids in a small volume (e.g., less than 100, less than 60 microliters) and that are substantially and/or effectively free of substances that inhibit assays used to test the DNA samples (e.g., PCR, INVADER, QuARTS assays, etc.) are provided. Such DNA samples find use in diagnostic assays that qualitatively detect the presence of, or quantitatively measure the activity, expression, or amount of, a gene, a gene variant (e.g., an allele), or a gene modification (e.g., methylation) present in a sample taken from a patient. For example, some cancers are correlated with the presence of particular mutant alleles or particular methylation states, and thus detecting and/or quantifying such mutant alleles or methylation states has predictive value in the diagnosis and treatment of cancer.

Many valuable genetic markers are present in extremely low amounts in samples and many of the events that produce such markers are rare. Consequently, even sensitive detection methods such as PCR require a large amount of DNA to provide enough of a low-abundance target to meet or supersede the detection threshold of the assay. Moreover, the presence of even low amounts of inhibitory substances compromise the accuracy and precision of these assays directed to detecting such low amounts of a target. Accordingly, provided herein are methods providing the requisite management of volume and concentration to produce such DNA samples.

In some embodiments, the sample comprises stool, tissue sample (e.g., pancreatic tissue), an organ secretion, CSF, saliva, blood, or urine. In some embodiments, the subject is human. Such samples can be obtained by any number of means known in the art, such as will be apparent to the skilled person. Cell free or substantially cell free samples can be obtained by subjecting the sample to various techniques known to those of skill in the art which include, but are not limited to, centrifugation and filtration. Although it is generally preferred that no invasive techniques are used to obtain the sample, it still may be preferable to obtain samples such as tissue homogenates, tissue sections, and biopsy specimens. The technology is not limited in the methods used to prepare the samples and provide a nucleic acid for testing. For example, in some embodiments, a DNA is isolated from a stool sample or from blood or from a plasma sample using direct gene capture, e.g., as detailed in U.S. Pat. Nos. 8,808,990 and 9,169,511, and in WO 2012/155072, or by a related method.

The analysis of markers can be carried out separately or simultaneously with additional markers within one test sample. For example, several markers can be combined into one test for efficient processing of multiple samples and for potentially providing greater diagnostic and/or prognostic accuracy. In addition, one skilled in the art would recognize the value of testing multiple samples (for example, at successive time points) from the same subject. Such testing of serial samples can allow the identification of changes in marker methylation states over time. Changes in methylation state, as well as the absence of change in methylation state, can provide useful information about the disease status that includes, but is not limited to, identifying the approximate time from onset of the event, the presence and amount of salvageable tissue, the appropriateness of drug therapies, the effectiveness of various therapies, and identification of the subject's outcome, including risk of future events. The analysis of biomarkers can be carried out in a variety of physical formats. For example, the use of microtiter plates or automation can be used to facilitate the processing of large numbers of test samples. Alternatively, single sample formats could be developed to facilitate immediate treatment and diagnosis in a timely fashion, for example, in ambulatory transport or emergency room settings.

It is contemplated that embodiments of the technology are provided in the form of a kit. The kits comprise embodiments of the compositions, devices, apparatuses, etc. described herein, and instructions for use of the kit. Such instructions describe appropriate methods for preparing an analyte from a sample, e.g., for collecting a sample and preparing a nucleic acid from the sample. Individual components of the kit are packaged in appropriate containers and packaging (e.g., vials, boxes, blister packs, ampules, jars, bottles, tubes, and the like) and the components are packaged together in an appropriate container (e.g., a box or boxes) for convenient storage, shipping, and/or use by the user of the kit. It is understood that liquid components (e.g., a buffer) may be provided in a lyophilized form to be reconstituted by the user. Kits may include a control or reference for assessing, validating, and/or assuring the performance of the kit. For example, a kit for assaying the amount of a nucleic acid present in a sample may include a control comprising a known concentration of the same or another nucleic acid for comparison and, in some embodiments, a detection reagent (e.g., a primer) specific for the control nucleic acid. The kits are appropriate for use in a clinical setting and, in some embodiments, for use in a user's home. The components of a kit, in some embodiments, provide the functionalities of a system for preparing a nucleic acid solution from a sample. In some embodiments, certain components of the system are provided by the user.

Various cancers are predicted by various combinations of markers, e.g., as identified by statistical techniques related to specificity and sensitivity of prediction. The technology provides methods for identifying predictive combinations and validated predictive combinations for some cancers.

### Methods

In some embodiments of the technology, methods are provided that comprise the following steps:
1) contacting a nucleic acid (e.g., genomic DNA, e.g., isolated from pancreatic tissue) obtained from the subject with at least one reagent or series of reagents that distinguishes between methylated and non-methylated CpG dinucleotides within at least one marker selected from a chromosomal region having an annotation recited in Table 1A, and
2) detecting PNET (e.g., afforded with a sensitivity of greater than or equal to 80% and a specificity of greater than or equal to 80%).

In some embodiments of the technology, methods are provided that comprise the following steps:
1) contacting a nucleic acid (e.g., genomic DNA, e.g., isolated from pancreatic tissue) obtained from the subject with at least one reagent or series of reagents that distinguishes between methylated and non-methylated CpG dinucleotides within at least one marker selected from a chromosomal region having an annotation selected from the group consisting of ANXA2, CACNA1C_A, CDHR2, FBXL16_B, GP1BB_A, GP1BB_C, HCN2, HPCAL1, LOC100129726, MAX.chr17.77788758-77788971, PDZD2, PTPRN2, RASSF3, RTN2, RUNDC3A, RXRA, SLC38A2, SPTBN4, SRRM3, STX10_B, TMC6_A, TSPO, CUX1, FAM78A, FNBP1, IER2, MOBKL2A, PNMAL2, STPR4_A, LGALS3, and MYO15B, and
2) detecting PNET (e.g., afforded with a sensitivity of greater than or equal to 80% and a specificity of greater than or equal to 80%).

In some embodiments of the technology, methods are provided that comprise the following steps:
1) contacting a nucleic acid (e.g., genomic DNA, e.g., isolated from a blood sample (e.g., plasma sample, whole blood sample, leukocyte sample, serum sample) obtained from the subject with at least one reagent or series of reagents that distinguishes between methylated and non-methylated CpG dinucleotides within at least one marker selected from a chromosomal region having an annotation recited in Table 2A, and
2) detecting PNET (e.g., afforded with a sensitivity of greater than or equal to 80% and a specificity of greater than or equal to 80%).

In some embodiments of the technology, methods are provided that comprise the following steps:
2) contacting a nucleic acid (e.g., genomic DNA, e.g., isolated from a blood sample (e.g., plasma sample, whole blood sample, leukocyte sample, serum sample) obtained from the subject with at least one reagent or series of reagents that distinguishes between methylated and non-methylated CpG dinucleotides within at least one marker selected from a chromosomal region having an annotation selected from the group consisting of ANXA2, CACNA1C_A, CDHR2, FBXL16_B, GP1BB_A, GP1BB_C, HCN2, HPCAL1, LOC100129726, MAX.chr17.77788758-77788971, PDZD2, PTPRN2, RASSF3, RTN2, RUNDC3A, RXRA, SLC38A2, SPTBN4, SRRM3, STX10_B, TMC6_A, TSPO, CUX1, FAM78A, FNBP1, IER2, MOBKL2A, PNMAL2, STPR4_A, LGALS3, and MYO15B, and

2) detecting PNET (e.g., afforded with a sensitivity of greater than or equal to 80% and a specificity of greater than or equal to 80%).

In some embodiments of the technology, methods are provided that comprise the following steps:
3) contacting a nucleic acid (e.g., genomic DNA, e.g., isolated from a blood sample (e.g., plasma sample, whole blood sample, leukocyte sample, serum sample) obtained from the subject with at least one reagent or series of reagents that distinguishes between methylated and non-methylated CpG dinucleotides within at least one marker selected from a chromosomal region having an annotation selected from the group consisting of SRRM3, HCN2, SPTBN4, TMC6_A, GP1BB_C, GP1BB_A, STX10_B, CACNA1C_A, CDHR2, PTPRN2, MAX.chr17.77788758.77788971, FBXL16_B, RTN2, HPCAL1, RASSF3, TSPO, RUNDC3A, SLC38A2, MAX.chr19.2478419.2478656, PDZD2, LOC100129726, CUX1, ANXA2, RXRA, S1PR4_A, FNBP1, FAM78A, IER2, PNMAL2, and MOBKL2A, and

2) detecting metastatic PNET (e.g., afforded with a sensitivity of greater than or equal to 80% and a specificity of greater than or equal to 80%).

In some embodiments of the technology, methods are provided that comprise the following steps:
4) contacting a nucleic acid (e.g., genomic DNA, e.g., isolated from a blood sample (e.g., plasma sample, whole blood sample, leukocyte sample, serum sample) obtained from the subject with at least one reagent or series of reagents that distinguishes between methylated and non-methylated CpG dinucleotides within at least one marker selected from a chromosomal region having an annotation selected from the group consisting of SRRM3, HCN2, SPTBN4, TMC6_A, GP1BB_C, GP1BB_A, STX10_B, CACNA1C_A, CDHR2, PTPRN2, MAX.chr17.77788758.77788971, FBXL16_B, RTN2, HPCAL1, RASSF3, TSPO, RUNDC3A, SLC38A2, MAX.chr19.2478419.2478656, PDZD2, LOC100129726, CUX1, ANXA2, RXRA, S1PR4_A, FNBP1, FAM78A, IER2, PNMAL2, and MOBKL2A, and

2) detecting lung NET (e.g., afforded with a sensitivity of greater than or equal to 80% and a specificity of greater than or equal to 80%).

In some embodiments of the technology, methods are provided that comprise the following steps:
5) contacting a nucleic acid (e.g., genomic DNA, e.g., isolated from a blood sample (e.g., plasma sample, whole blood sample, leukocyte sample, serum sample) obtained from the subject with at least one reagent or series of reagents that distinguishes between methylated and non-methylated CpG dinucleotides within at least one marker selected from a chromosomal region having an annotation selected from the group consisting of SRRM3, HCN2, SPTBN4, TMC6_A, GP1BB_C, GP1BB_A, STX10_B, CACNA1C_A, CDHR2, PTPRN2, MAX.chr17.77788758.77788971, FBXL16_B, RTN2, HPCAL1, RASSF3, TSPO, RUNDC3A, SLC38A2, MAX.chr19.2478419.2478656, PDZD2, LOC100129726, CUX1, ANXA2, RXRA, S1PR4_A, FNBP1, FAM78A, IER2, PNMAL2, and MOBKL2A, and

2) detecting small bowel NET (e.g., afforded with a sensitivity of greater than or equal to 80% and a specificity of greater than or equal to 80%).

In some embodiments of the technology, methods are provided that comprise the following steps:

1) measuring a methylation level for one or more genes in a biological sample of a human individual through treating genomic DNA in the biological sample with a reagent that modifies DNA in a methylation-specific manner (e.g., wherein the reagent is a bisulfite reagent, a methylation-sensitive restriction enzyme, or a methylation-dependent restriction enzyme), wherein the one or more genes is selected from ANXA2, CACNA1C_A, CDHR2, FBXL16_B, GP1BB_A, GP1BB_C, HCN2, HPCAL1, LOC100129726, MAX.chr17.77788758-77788971, PDZD2, PTPRN2, RASSF3, RTN2, RUNDC3A, RXRA, SLC38A2, SPTBN4, SRRM3, STX10_B, TMC6_A, TSPO, CUX1, FAM78A, FNBP1, IER2, MOBKL2A, PNMAL2, S1PR4_A, LGALS3, and MYO15B;

2) amplifying the treated genomic DNA using a set of primers for the selected one or more genes; and

3) determining the methylation level of the one or more genes by polymerase chain reaction, nucleic acid sequencing, mass spectrometry, methylation-specific nuclease, mass-based separation, and target capture.

In some embodiments of the technology, methods are provided that comprise the following steps:

1) measuring an amount of at least one methylated marker gene in DNA from the sample, wherein the one or more genes is selected from ANXA2, CACNA1C_A, CDHR2, FBXL16_B, GP1BB_A, GP1BB_C, HCN2, HPCAL1, LOC100129726, MAX.chr17.77788758-77788971, PDZD2, PTPRN2, RASSF3, RTN2, RUNDC3A, RXRA, SLC38A2, SPTBN4, SRRM3, STX10_B, TMC6_A, TSPO, CUX1, FAM78A, FNBP1, IER2, MOBKL2A, PNMAL2, STPR4_A, LGALS3, and MYO15B;

2) measuring the amount of at least one reference marker in the DNA; and

3) calculating a value for the amount of the at least one methylated marker gene measured in the DNA as a percentage of the amount of the reference marker gene measured in the DNA, wherein the value indicates the amount of the at least one methylated marker DNA measured in the sample.

In some embodiments of the technology, methods are provided that comprise the following steps:

1) measuring a methylation level of a CpG site for one or more genes in a biological sample of a human individual through treating genomic DNA in the biological sample with bisulfite a reagent capable of modifying DNA in a methylation-specific manner (e.g., a methylation-sensitive restriction enzyme, a methylation-dependent restriction enzyme, and a bisulfite reagent);

2) amplifying the modified genomic DNA using a set of primers for the selected one or more genes; and

3) determining the methylation level of the CpG site by methylation-specific PCR, quantitative methylation-specific PCR, methylation-sensitive DNA restriction enzyme analysis, quantitative bisulfite pyrosequencing, or bisulfite genomic sequencing PCR;
wherein the one or more genes is selected from ANXA2, CACNA1C_A, CDHR2, FBXL16_B, GP1BB_A, GP1BB_C, HCN2, HPCAL1, LOC100129726, MAX.chr17.77788758-77788971, PDZD2, PTPRN2, RASSF3, RTN2, RUNDC3A, RXRA, SLC38A2, SPTBN4, SRRM3, STX10_B, TMC6_A, TSPO, CUX1, FAM78A, FNBP1, IER2, MOBKL2A, PNMAL2, STPR4_A, LGALS3, and MYO15B.

Preferably, the sensitivity for such methods is from about 70% to about 100%, or from about 80% to about 90%, or from about 80% to about 85%. Preferably, the specificity is from about 70% to about 100%, or from about 80% to about 90%, or from about 80% to about 85%.

Genomic DNA may be isolated by any means, including the use of commercially available kits. Briefly, wherein the DNA of interest is encapsulated in by a cellular membrane the biological sample must be disrupted and lysed by enzymatic, chemical or mechanical means. The DNA solution may then be cleared of proteins and other contaminants, e.g., by digestion with proteinase K. The genomic DNA is then recovered from the solution. This may be carried out by means of a variety of methods including salting out, organic extraction, or binding of the DNA to a solid phase support. The choice of method will be affected by several factors including time, expense, and required quantity of DNA. All clinical sample types comprising neoplastic matter or pre-neoplastic matter are suitable for use in the present method, e.g., cell lines, histological slides, biopsies, paraffin-embedded tissue, body fluids, stool, breast tissue, endometrial tissue, leukocytes, colonic effluent, urine, blood plasma, blood serum, whole blood, isolated blood cells, cells isolated from the blood, and combinations thereof.

The technology is not limited in the methods used to prepare the samples and provide a nucleic acid for testing. For example, in some embodiments, a DNA is isolated from a stool sample or from blood or from a plasma sample using direct gene capture, e.g., as detailed in U.S. Pat. Appl. Ser. No. 61/485,386 or by a related method.

The genomic DNA sample is then treated with at least one reagent, or series of reagents, that distinguishes between methylated and non-methylated CpG dinucleotides within at least one marker comprising a DMR (e.g., DMR 1-198 e.g., as provided by Tables 1A and 2A).

In some embodiments, the reagent converts cytosine bases which are unmethylated at the 5′-position to uracil, thymine, or another base which is dissimilar to cytosine in terms of hybridization behavior. However in some embodiments, the reagent may be a methylation sensitive restriction enzyme.

In some embodiments, the genomic DNA sample is treated in such a manner that cytosine bases that are unmethylated at the 5′ position are converted to uracil, thymine, or another base that is dissimilar to cytosine in terms of hybridization behavior. In some embodiments, this treatment is carried out with bisulfite (hydrogen sulfite, disulfite) followed by alkaline hydrolysis.

The treated nucleic acid is then analyzed to determine the methylation state of the target gene sequences (at least one gene, genomic sequence, or nucleotide from a marker comprising a DMR, e.g., at least one DMR chosen from DMR 1-198, e.g., as provided in Tables TA and 2A). The method of analysis may be selected from those known in the art, including those listed herein, e.g., QuARTS and MSP as described herein.

Aberrant methylation, more specifically hypermethylation of a marker comprising a DMR (e.g., DMR 1-66, e.g., as provided by Table 3) is associated with PNET.

The technology relates to the analysis of any sample associated with an PNET. For example, in some embodiments the sample comprises a tissue and/or biological fluid obtained from a patient. In some embodiments, the sample comprises a secretion. In some embodiments, the sample comprises blood, serum, plasma, gastric secretions, pancreatic juice, a gastrointestinal biopsy sample, microdissected cells from a breast biopsy, and/or cells recovered from stool. In some embodiments, the sample comprises pancreatic tissue. In some embodiments, the subject is human. The sample may include cells, secretions, or tissues from the endometrium, breast, liver, bile ducts, pancreas, stomach, colon, rectum, esophagus, small intestine, appendix, duodenum, polyps, gall bladder, anus, and/or peritoneum. In some embodiments, the sample comprises cellular fluid, ascites, urine, feces, pancreatic fluid, fluid obtained during endoscopy, blood, mucus, or saliva. In some embodiments, the sample is a stool sample. In some embodiments, the sample is a pancreatic tissue sample.

Such samples can be obtained by any number of means known in the art, such as will be apparent to the skilled person. For instance, urine and fecal samples are easily attainable, while blood, ascites, serum, or pancreatic fluid samples can be obtained parenterally by using a needle and syringe, for instance. Cell free or substantially cell free samples can be obtained by subjecting the sample to various techniques known to those of skill in the art which include, but are not limited to, centrifugation and filtration. Although it is generally preferred that no invasive techniques are used to obtain the sample, it still may be preferable to obtain samples such as tissue homogenates, tissue sections, and biopsy specimens

In some embodiments, the technology relates to a method for treating a patient (e.g., a patient with PNET, with early stage PNET, or who may develop PNET), the method comprising determining the methylation state of one or more DMR as provided herein and administering a treatment to the patient based on the results of determining the methylation state. The treatment may be administration of a pharmaceutical compound, a vaccine, an immunotherapy, performing a surgery, imaging the patient, performing another test. Preferably, said use is in a method of clinical screening, a method of prognosis assessment, a method of monitoring the results of therapy, a method to identify patients most likely to respond to a particular therapeutic treatment, a method of imaging a patient or subject, and a method for drug screening and development.

In some embodiments of the technology, a method for diagnosing a PNET in a subject is provided. The terms “diagnosing” and “diagnosis” as used herein refer to methods by which the skilled artisan can estimate and even determine whether or not a subject is suffering from a given disease or condition or may develop a given disease or condition in the future. The skilled artisan often makes a diagnosis on the basis of one or more diagnostic indicators, such as for example a biomarker (e.g., a DMR as disclosed herein), the methylation state of which is indicative of the presence, severity, or absence of the condition.

Along with diagnosis, clinical cancer prognosis relates to determining the aggressiveness of the cancer and the likelihood of tumor recurrence to plan the most effective therapy. If a more accurate prognosis can be made or even a potential risk for developing the cancer can be assessed, appropriate therapy, and in some instances less severe therapy for the patient can be chosen. Assessment (e.g., determining methylation state) of cancer biomarkers is useful to separate subjects with good prognosis and/or low risk of developing cancer who will need no therapy or limited therapy from those more likely to develop cancer or suffer a recurrence of cancer who might benefit from more intensive treatments.

As such, “making a diagnosis” or “diagnosing”, as used herein, is further inclusive of determining a risk of developing cancer or determining a prognosis, which can provide for predicting a clinical outcome (with or without medical treatment), selecting an appropriate treatment (or whether treatment would be effective), or monitoring a current treatment and potentially changing the treatment, based on the measure of the diagnostic biomarkers (e.g., DMR) disclosed herein. Further, in some embodiments of the presently disclosed subject matter, multiple determination of the biomarkers over time can be made to facilitate diagnosis and/or prognosis. A temporal change in the biomarker can be used to predict a clinical outcome, monitor the progression of a PNET, and/or monitor the efficacy of appropriate therapies directed against the cancer. In such an embodiment for example, one might expect to see a change in the methylation state of one or more biomarkers (e.g., DMR) disclosed herein (and potentially one or more additional biomarker(s), if monitored) in a biological sample over time during the course of an effective therapy.

The presently disclosed subject matter further provides in some embodiments a method for determining whether to initiate or continue prophylaxis or treatment of a cancer in a subject. In some embodiments, the method comprises providing a series of biological samples over a time period from the subject; analyzing the series of biological samples to determine a methylation state of at least one biomarker disclosed herein in each of the biological samples; and comparing any measurable change in the methylation states of one or more of the biomarkers in each of the biological samples. Any changes in the methylation states of biomarkers over the time period can be used to predict risk of developing cancer, predict clinical outcome, determine whether to initiate or continue the prophylaxis or therapy of the cancer, and whether a current therapy is effectively treating the cancer. For example, a first time point can be selected prior to initiation of a treatment and a second time point can be selected at some time after initiation of the treatment. Methylation states can be measured in each of the samples taken from different time points and qualitative and/or quantitative differences noted. A change in the methylation states of the biomarker levels from the different samples can be correlated with PNET risk, prognosis, determining treatment efficacy, and/or progression of the cancer in the subject.

In preferred embodiments, the methods and compositions of the invention are for treatment or diagnosis of disease at an early stage, for example, before symptoms of the disease appear. In some embodiments, the methods and compositions of the invention are for treatment or diagnosis of disease at a clinical stage.

As noted, in some embodiments, multiple determinations of one or more diagnostic or prognostic biomarkers can be made, and a temporal change in the marker can be used to determine a diagnosis or prognosis. For example, a diagnostic marker can be determined at an initial time, and again at a second time. In such embodiments, an increase in the marker from the initial time to the second time can be diagnostic of a particular type or severity of cancer, or a given prognosis. Likewise, a decrease in the marker from the initial time to the second time can be indicative of a particular type or severity of cancer, or a given prognosis. Furthermore, the degree of change of one or more markers can be related to the severity of the cancer and future adverse events. The skilled artisan will understand that, while in certain embodiments comparative measurements can be made of the same biomarker at multiple time points, one can also measure a given biomarker at one time point, and a second biomarker at a second time point, and a comparison of these markers can provide diagnostic information.

As used herein, the phrase “determining the prognosis” refers to methods by which the skilled artisan can predict the course or outcome of a condition in a subject. The term “prognosis” does not refer to the ability to predict the course or outcome of a condition with 100% accuracy, or even that a given course or outcome is predictably more or less likely to occur based on the methylation state of a biomarker (e.g., a DMR). Instead, the skilled artisan will understand that the term “prognosis” refers to an increased probability that a certain course or outcome will occur; that is, that a course or outcome is more likely to occur in a subject exhibiting a given condition, when compared to those individuals not exhibiting the condition. For example, in individuals not exhibiting the condition (e.g., having a normal methylation state of one or more DMR), the chance of a given outcome (e.g., suffering from a PNET) may be very low.

In some embodiments, a statistical analysis associates a prognostic indicator with a predisposition to an adverse outcome. For example, in some embodiments, a methylation state different from that in a normal control sample obtained from a patient who does not have a cancer can signal that a subject is more likely to suffer from a cancer than subjects with a level that is more similar to the methylation state in the control sample, as determined by a level of statistical significance. Additionally, a change in methylation state from a baseline (e.g., “normal”) level can be reflective of subject prognosis, and the degree of change in methylation state can be related to the severity of adverse events. Statistical significance is often determined by comparing two or more populations and determining a confidence interval and/or a p value. See, e.g., Dowdy and Wearden, Statistics for Research, John Wiley & Sons, New York, 1983, incorporated herein by reference in its entirety. Exemplary confidence intervals of the present subject matter are 90%, 95%, 97.5%, 98%, 99%, 99.5%, 99.9% and 99.99%, while exemplary p values are 0.1, 0.05, 0.025, 0.02, 0.01, 0.005, 0.001, and 0.0001.

In other embodiments, a threshold degree of change in the methylation state of a prognostic or diagnostic biomarker disclosed herein (e.g., a DMR) can be established, and the degree of change in the methylation state of the biomarker in a biological sample is simply compared to the threshold degree of change in the methylation state. A preferred threshold change in the methylation state for biomarkers provided herein is about 5%, about 10%, about 15%, about 20%, about 25%, about 30%, about 50%, about 75%, about 100%, and about 150%. In yet other embodiments, a “nomogram” can be established, by which a methylation state of a prognostic or diagnostic indicator (biomarker or combination of biomarkers) is directly related to an associated disposition towards a given outcome. The skilled artisan is acquainted with the use of such nomograms to relate two numeric values with the understanding that the uncertainty in this measurement is the same as the uncertainty in the marker concentration because individual sample measurements are referenced, not population averages.

In some embodiments, a control sample is analyzed concurrently with the biological sample, such that the results obtained from the biological sample can be compared to the results obtained from the control sample. Additionally, it is contemplated that standard curves can be provided, with which assay results for the biological sample may be compared. Such standard curves present methylation states of a biomarker as a function of assay units, e.g., fluorescent signal intensity, if a fluorescent label is used. Using samples taken from multiple donors, standard curves can be provided for control methylation states of the one or more biomarkers in normal tissue, as well as for “at-risk” levels of the one or more biomarkers in tissue taken from donors with metaplasia or from donors with a PNET. In certain embodiments of the method, a subject is identified as having metaplasia upon identifying an aberrant methylation state of one or more DMR provided herein in a biological sample obtained from the subject. In other embodiments of the method, the detection of an aberrant methylation state of one or more of such biomarkers in a biological sample obtained from the subject results in the subject being identified as having cancer.

The analysis of markers can be carried out separately or simultaneously with additional markers within one test sample. For example, several markers can be combined into one test for efficient processing of a multiple of samples and for potentially providing greater diagnostic and/or prognostic accuracy. In addition, one skilled in the art would recognize the value of testing multiple samples (for example, at successive time points) from the same subject. Such testing of serial samples can allow the identification of changes in marker methylation states over time. Changes in methylation state, as well as the absence of change in methylation state, can provide useful information about the disease status that includes, but is not limited to, identifying the approximate time from onset of the event, the presence and amount of salvageable tissue, the appropriateness of drug therapies, the effectiveness of various therapies, and identification of the subject's outcome, including risk of future events.

The analysis of biomarkers can be carried out in a variety of physical formats. For example, the use of microtiter plates or automation can be used to facilitate the processing of large numbers of test samples. Alternatively, single sample formats could be developed to facilitate immediate treatment and diagnosis in a timely fashion, for example, in ambulatory transport or emergency room settings.

In some embodiments, the subject is diagnosed as having a PNET if, when compared to a control methylation state, there is a measurable difference in the methylation state of at least one biomarker in the sample. Conversely, when no change in methylation state is identified in the biological sample, the subject can be identified as not having PNET, not being at risk for the cancer, or as having a low risk of the cancer. In this regard, subjects having the cancer or risk thereof can be differentiated from subjects having low to substantially no cancer or risk thereof. Those subjects having a risk of developing a PNET can be placed on a more intensive and/or regular screening schedule, including endoscopic surveillance. On the other hand, those subjects having low to substantially no risk may avoid being subjected to additional testing for PNET (e.g., invasive procedure), until such time as a future screening, for example, a screening conducted in accordance with the present technology, indicates that a risk of PNET has appeared in those subjects.

As mentioned above, depending on the embodiment of the method of the present technology, detecting a change in methylation state of the one or more biomarkers can be a qualitative determination or it can be a quantitative determination. As such, the step of diagnosing a subject as having, or at risk of developing, an PNET indicates that certain threshold measurements are made, e.g., the methylation state of the one or more biomarkers in the biological sample varies from a predetermined control methylation state. In some embodiments of the method, the control methylation state is any detectable methylation state of the biomarker. In other embodiments of the method where a control sample is tested concurrently with the biological sample, the predetermined methylation state is the methylation state in the control sample. In other embodiments of the method, the predetermined methylation state is based upon and/or identified by a standard curve. In other embodiments of the method, the predetermined methylation state is a specific state or range of state. As such, the predetermined methylation state can be chosen, within acceptable limits that will be apparent to those skilled in the art, based in part on the embodiment of the method being practiced and the desired specificity, etc.

Further with respect to diagnostic methods, a preferred subject is a vertebrate subject. A preferred vertebrate is warm-blooded; a preferred warm-blooded vertebrate is a mammal. A preferred mammal is most preferably a human. As used herein, the term “subject’ includes both human and animal subjects. Thus, veterinary therapeutic uses are provided herein. As such, the present technology provides for the diagnosis of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Thus, also provided is the diagnosis and treatment of livestock, including, but not limited to, domesticated swine, ruminants, ungulates, horses (including race horses), and the like.

The presently-disclosed subject matter further includes a system for diagnosing a PNET in a subject. The system can be provided, for example, as a commercial kit that can be used to screen for a risk of a PNET or diagnose a PNET cancer in a subject from whom a biological sample has been collected. An exemplary system provided in accordance with the present technology includes assessing the methylation state of a DMR as provided in Tables 1A and 2A.

### EXAMPLES

### Example I

### Materials and Methods

Tissue and blood were obtained from Mayo Clinic biospecimen repositories with institutional IRB oversight. Samples were chosen with strict adherence to subject research authorization and inclusion/exclusion criteria (detailed in the study protocol). Cases consisted of 28 solid and 16 cystic pancreatic neuroendocrine tumors (PNETs). Controls included 13 non-neoplastic pancreas tissue and 18 buffy coat samples from cancer free patients. Tissue samples from patients with prior history of pancreatic ductal adenocarcinoma (PDAC), those who has received chemotherapy class drugs in the past 6 months or had therapeutic radiation to the abdomen were excluded from the study. Tissues were macro-dissected and histology reviewed by an expert pathologist. Samples were age matched, randomized, and blinded. DNA from tissues and blood samples were purified using the Qiagen QIAmp FFPE tissue kit and QIAamp DNA Blood Mini kit (Qiagen, Valencia Calif.), respectively. DNA was re-purified with AMPure XP beads (Beckman-Coulter, Brea Calif.) and quantified by PicoGreen (Thermo-Fisher, Waltham Mass.). DNA integrity was assessed using qPCR.

RRBS sequencing libraries were prepared using the NuGEN Ovation RRBS Methyl-Seq kit with modifications (Tecan Genomics, Redwood City, Calif.). Samples were combined in a 4-plex format and sequenced by the Mayo Genomics Facility on the Illumina HiSeq 4000 instrument (Illumina, San Diego Calif.). Reads were processed by Illumina pipeline modules for image analysis and base calling. Secondary analysis was performed using SAAP-RRBS, a Mayo developed bioinformatics suite. Briefly, reads were cleaned-up using Trim-Galore and aligned to the GRCh37/hg19 reference genome build with BSMAP. Methylation ratios were determined by calculating C/(C+T) or conversely, G/(G+A) for reads mapping to reverse strand, for CpGs with coverage ≥10× and base quality score ≥20.

Individual CpGs were ranked by hypermethylation ratio, namely the number of methylated cytosines at a given locus over the total cytosine count at that site. For cases, the ratios were required to be ≥0.20 (20%); for tissue controls, ≤0.05 (5%) tissue vs tissue analysis; ≥0.20 (20%) tissue vs buffy coat; for buffy coat controls, ≤0.01 (1%). CpG hypermethylation was defined as least 20% methylation in cases compared to ≤5% in tissue controls or; ≤1% for buffy coat controls. CpGs which did not meet these criteria were discarded. Subsequently, candidate CpGs were binned by genomic location into DMRs (differentially methylated regions) ranging from approximately 40-220 bp with a minimum cut-off of 5 CpGs per region. DMRs with excessively high CpG density (>30%) were excluded to avoid GC-related amplification problems in the validation phase. Two analyses were performed comparing PNET tissue vs normal tissue controls and PNET tissue vs buffy coat controls. Following regression, the two sets of DMRs were ranked by p-value, area under the receiver operating characteristic curve (AUC) and fractional methylation ratio between cases and all controls. No adjustments for false discovery were made during this phase as independent validation was planned a priori.

For each candidate region, a 2-D matrix was created which compared individual CpGs in a sample-to-sample fashion for both cases and controls. These CpG matrices were then compared back to the reference sequence to assess whether genomically contiguous methylation sites had been discarded during the initial filtering. From this subset of regions, final selections required coordinated and contiguous hypermethylation (in cases) of individual CpGs across the DMR sequence on a per sample level. Conversely, control samples had to have at least 5-fold less methylation than cases and the CpG pattern had to be more random and less coordinated. At least 10% of cancer samples were required to have at least a 50% hypermethylation ratio for every CpG site within the DMR.

In a separate but complementary analysis, experiments utilized proprietary DMR identification pipeline and regression package to derive DMRs based on average methylation values of the CpG dinucleotides. The differences in average methylation percentage were compared between PNET cases, tissue controls and buffy coat controls; a tiled reading frame within 100 base pairs of each mapped CpG was used to identify DMRs where control methylation was <5%; DMRs were only analyzed if the total depth of coverage was 10 reads per subject on average and the variance across subgroups was >0.

Following regression, DMRs were ranked by p-value, area under the receiver operating characteristic curve (AUC) and fold-change difference between cases and all controls. No adjustments for false discovery were made during this phase as independent validation was planned a priori.

A subset of the DMRs was chosen for further development. The criteria were primarily the logistic-derived area under the ROC curve metric which provides a performance assessment of the discriminant potential of the region. An AUC of 0.90 was chosen as the cut-off (0.95 for the case vs buffy coat comparison). In addition, the methylation fold-change ratio (average cancer hypermethylation ratio/average control hypermethylation ratio) was calculated and a lower limit of 5 was employed for tissue vs tissue comparisons and 100 for the tissue vs buffy coat comparisons. P values were required to be less than 0.01. DMRs had to be listed in both the average and individual CpG selection processes. Quantitative methylation specific PCR (qMSP) primers were designed for candidate regions using MethPrimer (see, Li L C and Dahiya R. Bioinformatics 2002 November; 18(11):1427-31) and QC checked on 20 ng (6250 equivalents) of positive and negative genomic methylation controls. Multiple annealing temperatures were tested for optimal discrimination. Validation was performed in two stages of qMSP. The first consisted of re-testing the sequenced DNA samples. This was done to verify that the DMRs were truly discriminant and not the result of over-fitting the extremely large next generation datasets. The second utilized a larger set of independent samples: 67 primary PNETs (50 solid, 17 cystic), 25 metastatic PNETs, 36 lung and 36 small bowel neuroendocrine tumors, 24 normal pancreatic control tissues, and 36 normal buffy coat samples.

Tissues were identified as before, with expert clinical and pathological review. DNA purification was performed using the Qiagen QIAmp FFPE tissue kit. The EZ-96 DNA Methylation kit (Zymo Research, Irvine Calif.) was used for the bisulfite conversion step. 10 ng of converted DNA (per marker) was amplified using SYBR Green detection on Roche 480 LightCyclers (Roche, Basel Switzerland). Serially diluted universal methylated genomic DNA (Zymo Research) was used as a quantitation standard. A CpG agnostic ACTB (0-actin) assay was used as an input reference and normalization control. Results were expressed as methylated copies (specific marker)/copies of ACTB.

Results were analyzed logistically for individual MDMs (methylated DNA marker) performance. For combinations of markers, two techniques were used. First, the rPart technique was applied to the entire MDM set and limited to combinations of 3 MDMs, upon which an rPart predicted probability of cancer was calculated. The second approach used random forest regression (rForest) which generated 500 individual rPart models that were fit to boot strap samples of the original data (roughly ⅔ of the data for training) and used to estimate the cross-validation error (⅓ of the data for testing) of the entire MDM panel and was repeated 500 times. to avoid spurious splits that either under- or overestimate the true cross-validation metrics. Results were then averaged across the 500 iterations.

### Results

Experiments utilized a proprietary methodology of sample preparation, sequencing, analyses pipelines, and filters (outlined in Methods) to identify and narrow PNET associated differentially methylated regions (DMRs) to those which could be queried and utilized in a clinical testing environment.

From the tissue-to-tissue analysis, 72 hypermethylated DMRs were identified (Table 1A and 1B). They included PNET specific regions as well as those regions that targeted a more universal cancer spectrum. The tissue to leukocyte (buffy coat) analysis yielded 126 hypermethylated tissue DMRs with less than 1% noise in WBCs (Table 2A and 2B). Individual AUCs for regions that met selection criteria ranged from 0.90-1.00 with many exceeding 0.95. The pNET tissue and buffy coat comparison yielded the most dramatic differences in methylation signal, due to the specific epigenetic nature and signature of the two cell types, whereas the tissue analysis comparing normal pancreas and pNET tissue less so, but there were several MDMs which exhibited high discrimination in both groups and were selected for subsequent validation

From the tissue and buffy marker groups, 33 candidates were chosen for an initial pilot. Methylation-specific PCR assays were developed and tested on two rounds of tissue samples; those that were sequenced (frozen) and larger independent cohorts (FFPE). Short amplicon primers (<150 bp) were designed to target the most discriminant CpGs with in a DMR and tested on controls to ensure that fully methylated fragments amplified robustly and in a linear fashion; that unmethylated and/or unconverted fragments did not amplify. The 66 primer sequences and annealing temperatures are listed in Table 3.

The results from stage one validation were analyzed logistically to determine AUC and fold change. The analyses for the tissue and buffy coat controls were run separately. Results are highlighted in Table 4. The blue shading indicates markers with AUCs in excess of 0.90. A number of assays were 100% discriminant in PNET from buffy coat samples and others were near perfect in the PNET vs control tissue analysis.

These results provided a rich source of highly performing candidates to take into independent sample testing. Of the original 33 assays, 31 were selected. Since the final PNET test is envisioned to be blood-based, the ability of these MDMs to discriminate from normal leukocyte-derived cfDNA is paramount. Therefore, selection was weighed heavily toward high performing MDMs when compared the buffy coat samples. Two markers were excluded as their AUCs were less than 0.90. The remaining 31 fell within the AUC range of 0.90-1.00. All of these assays demonstrated high analytical performance—linearity, efficiency, sequence specificity (assessed using melt curve analysis), and strong amplification.

In round 2 validation, as in the previous step, the entire sample and marker set was run in one batch. ˜10 ng of FFPE-derived sample DNA was run per marker—310 total. PNET vs normal tissue and buffy coat results for individual MDMs are listed in Table 5. Table 5A shows AUC for PNET tissue, cystic PNET tissue, solid PNET tissue, metastatic PNET tissue versus normal pancreatic tissue, and PNET tissue versus metastatic PNET tissue. Table 5B shows AUC for small bowel neuroendocrine tissue (NET) and lung NET versus PNET tissue. Table 5C shows AUC for metastatic PNET tissue, lung NET, and small bowel NET versus buffy coat. On receiver operator characteristics analyses of individual marker candidates, best fit AUCs for the PNET vs control tissue comparison ranged from 0.51 to 0.98. For the PNET vs buffy coat comparison, the AUC range was 0.91-1.0. Median AUCs were 0.88 and 0.99, respectively. Four MDMs (SRRM3, HCN2, SPTBN4 and TMC6_A) achieved individual cross validated AUCs ≥0.95 (Table 6). These MDMs were similarly discriminant in metastatic PNET tissue and in primary lung and small bowel NETs. Three out of these 4 MDMs perfectly differentiated PNET tissue from buffy coat with AUC of 1 and may be ideally suited for further development of a blood-based assay.

In sum, whole methylome sequencing, stringent filtering criteria, and biological validation yielded outstanding candidate MDMs for pancreatic neuroendocrine tumors. Moreover, these MDMs also differentiated metastatic PNETs from normal pancreas tissue.

TABLE 1A

| | | Chromo- | DMR Start- |
|---|---|---|---|
| DMR | Gene | some | End Positions |
| No. | Annotation | No. | (GRCh37/hg19) |
| 1 | ADCY4 | 14 | 24808742-24808909 |
| 2 | ANKHD1-EIF4EBP3 | 5 | 139927684-139927755 |
| 3 | ANXA2 | 15 | 60690826-60690959 |
| 4 | BTBD17 | 17 | 72352858-72353448 |
| 5 | C7orf23 | 7 | 86848467-86848674 |
| 6 | CACNA1C_A | 12 | 2692333-2692493 |
| 7 | CCDC102A | 16 | 57571027-57571105 |
| 8 | CDHR2 | 5 | 175969650-175969749 |
| 9 | CTBP2 | 10 | 126850434-126850576 |
| 10 | DIDO1 | 20 | 61560373-61560657 |
| 11 | EEF1A2 | 20 | 62119620-62119812 |
| 12 | ENO3 | 17 | 4853728-4853800 |
| 13 | FBXL16_A | 16 | 744986-745216 |
| 14 | FBXL16_B | 16 | 750628-750715 |
| 15 | FLJ22184 | 19 | 7934721-7934770 |
| 16 | FLOT1 | 6 | 30711524-30711803 |
| 17 | GP1BB_A | 22 | 19710945-19711074 |
| 18 | GP1BB_B | 22 | 19711446-19711516 |
| 19 | GP1BB_C | 22 | 19711681-19711850 |
| 20 | HCN2 | 19 | 591692-591781 |
| 21 | HPCAL1 | 2 | 10444412-10444495 |
| 22 | IZUMO1 | 19 | 49250409-49250467 |
| 23 | JSRP1_A | 19 | 2252384-2252398 |
| 24 | JSRP1_B | 19 | 2253163-2253319 |
| 25 | KCNH6 | 17 | 61615733-61615864 |
| 26 | LGALS3 | 14 | 55595729-55595844 |
| 27 | LIN28A | 1 | 26735086-26735256 |
| 28 | LMO4 | 1 | 87796247-87796350 |
| 29 | LOC100129726 | 2 | 43452130-43452585 |
| 30 | LTBP4 | 19 | 41116418-41116462 |
| 31 | MAST1 | 19 | 12978384-12978558 |
| 32 | MAX.chr11.518965- | 11 | 518965-519004 |
| | 519004 | | |
| 33 | MAX.chr14.69283008- | 14 | 69283008-69283105 |
| | 69283105 | | |
| 34 | MAX.chr15.76638902- | 15 | 76638902-76638974 |
| | 76638974 | | |
| 35 | MAX.chr17.2627804- | 17 | 2627804-2627879 |
| | 2627879 | | |
| 36 | MAX.chr17.2659373- | 17 | 2659373-2659452 |
| | 2659452 | | |
| 37 | MAX.chr17.42357220- | 17 | 42357220-42357323 |
| | 42357323 | | |
| 38 | MAX.chr17.77788758- | 17 | 77788758-77788971 |
| | 77788971 | | |
| 39 | MAX.chr19.13266472- | 19 | 13266472-13266581 |
| | 13266581 | | |
| 40 | MAX.chr19.2478419- | 19 | 2478419-2478656 |
| | 2478656 | | |
| 41 | MAX.chr22.25678201- | 22 | 25678201-25678290 |
| | 25678290 | | |
| 42 | MAX.chr3.127176070- | 3 | 127176070-127176139 |
| | 127176139 | | |
| 43 | MAX.chr9.137028591- | 9 | 137028591-137028864 |
| | 137028864 | | |
| 44 | MT1G | 16 | 56701913-56702151 |
| 45 | MYO15B | 17 | 73584898-73585121 |
| 46 | NCRNA00245 | 10 | 77164134-77164642 |
| 47 | OCA2 | 15 | 28339873-28340223 |
| 48 | PDE2A | 11 | 72301460-72301582 |
| 49 | PDZD2 | 5 | 31855237-31855491 |
| 50 | PHLDB3 | 19 | 43979342-43979600 |
| 51 | PPARGC1B | 5 | 149111737-149111884 |
| 52 | PTPRN2 | 7 | 157484509-157484663 |
| 53 | RASSF3 | 12 | 65004980-65005539 |
| 54 | RTN2 | 19 | 45996433-45996499 |
| 55 | RUNDC3A | 17 | 42392883-42393032 |
| 56 | RXRA | 9 | 137217099-137217558 |
| 57 | SAMD11 | 1 | 861287-861369 |
| 58 | SLC38A2 | 12 | 46767122-46767329 |
| 59 | SLC38A3 | 3 | 50243435-50243504 |
| 60 | SLC8A2 | 19 | 47933463-47933678 |
| 61 | SPTBN4 | 19 | 41060185-41060270 |
| 62 | SRRM3 | 7 | 75896582-75896785 |
| 63 | STX10_A | 19 | 13265725-13265813 |
| 64 | STX10_B | 19 | 13265939-13266255 |
| 65 | SYT5 | 19 | 55684965-55685063 |
| 66 | SYT7 | 11 | 61322823-61322904 |
| 67 | TGIF2 | 20 | 35203463-35203676 |
| 68 | TMC6_A | 17 | 76123640-76123768 |
| 69 | TMC6_B | 17 | 76124136-76124298 |
| 70 | TMEM145 | 19 | 42818005-42818094 |
| 71 | TNKS1BP1 | 11 | 57078772-57078977 |
| 72 | TSPO | 22 | 43548112-43548218 |

TABLE 1B

| | | Area | | |
|---|---|---|---|---|
| DMR | Gene | Under | Fold- | |
| No. | Annotation | Curve | Change | p-value |
| 1 | ADCY4 | 0.9156 | 6.628 | 0.0001779 |
| 2 | ANKHD1-EIF4EBP3 | 0.9502 | 7.534 | 2.84E−06 |
| 3 | ANXA2 | 0.9106 | 6.497 | 0.0002795 |
| 4 | BTBD17 | 0.9356 | 13.09 | 1.42E−06 |
| 5 | C7orf23 | 0.9606 | 44.2 | 0.00953 |
| 6 | CACNA1C_A | 0.9219 | 26.87 | 0.0006922 |
| 7 | CCDC102A | 0.9462 | 7.609 | 3.41E−05 |
| 8 | CDHR2 | 0.9431 | 25.78 | 0.00822 |
| 9 | CTBP2 | 0.9623 | 11.93 | 6.45E−06 |
| 10 | DIDO1 | 0.9034 | 6.459 | 0.005633 |
| 11 | EEF1A2 | 0.9811 | 23.6 | 1.08E−05 |
| 12 | ENO3 | 0.937 | 12.57 | 5.35E−05 |
| 13 | FBXL16_A | 0.9577 | 10.56 | 1.54E−06 |
| 14 | FBXL16_B | 0.9528 | 36.61 | 1.39E−05 |
| 15 | FLJ22184 | 0.927 | 10.86 | 3.43E−06 |
| 16 | FLOT1 | 0.8974 | 19.06 | 0.0009995 |
| 17 | GP1BB_A | 0.9233 | 9.225 | 3.04E−05 |
| 18 | GP1BB_B | 0.8981 | 12.2 | 5.42E−06 |
| 19 | GP1BB_C | 0.9487 | 13.51 | 2.24E−06 |
| 20 | HCN2 | 0.9583 | 48.06 | 4.15E−05 |
| 21 | HPCAL1 | 0.9545 | 27.51 | 0.00094 |
| 22 | IZUMO1 | 0.9484 | 13.03 | 9.94E−08 |
| 23 | JSRP1_A | 0.9122 | 7.722 | 8.39E−05 |
| 24 | JSRP1_B | 0.898 | 10.98 | 1.58E−05 |
| 25 | KCNH6 | 0.9081 | 20.38 | 1.47E−06 |
| 26 | LGALS3 | 0.9557 | 21.06 | 0.00266 |
| 27 | LIN28A | 0.9568 | 19.93 | 7.84E−07 |
| 28 | LMO4 | 0.9744 | 35.78 | 9.11E−06 |
| 29 | LOC100129726 | 0.9423 | 68.4 | 0.007458 |
| 30 | LTBP4 | 0.8958 | 5.247 | 0.0002122 |
| 31 | MAST1 | 0.9757 | 15.32 | 2.72E−07 |
| 32 | MAX.chr11.518965- | 0.8986 | 7.527 | 7.00E−05 |
| | 519004 | | | |
| 33 | MAX.chr14.69283008- | 0.9207 | 8.658 | 0.004686 |
| | 69283105 | | | |
| 34 | MAX.chr15.76638902- | 0.9051 | 7.104 | 0.0001099 |
| | 76638974 | | | |
| 35 | MAX.chr17.2627804- | 0.9649 | 12.9 | 9.25E−08 |
| | 2627879 | | | |
| 36 | MAX.chr17.2659373- | 0.9475 | 28.74 | 0.000786 |
| | 2659452 | | | |
| 37 | MAX.chr17.42357220- | 0.9628 | 15.42 | 3.36E−07 |
| | 42357323 | | | |
| 38 | MAX.chr17.77788758- | 0.9286 | 30.46 | 0.001833 |
| | 77788971 | | | |
| 39 | MAX.chr19.13266472- | 0.9235 | 67.09 | 0.002876 |
| | 13266581 | | | |
| 40 | MAX.chr19.2478419- | 0.9306 | 16.61 | 5.56E−05 |
| | 2478656 | | | |
| 41 | MAX.chr22.25678201- | 0.9118 | 13.14 | 3.83E−05 |
| | 25678290 | | | |
| 42 | MAX.chr3.127176070- | 0.9183 | 6.631 | 3.78E−05 |
| | 127176139 | | | |
| 43 | MAX.chr9.137028591- | 0.9514 | 12.79 | 4.75E−05 |
| | 137028864 | | | |
| 44 | MT1G | 0.926 | 8.244 | 7.36E−05 |
| 45 | MYO15B | 0.9069 | 24.67 | 0.0006389 |
| 46 | NCRNA00245 | 0.9206 | 10.96 | 0.000104 |
| 47 | OCA2 | 0.9137 | 14.03 | 0.0006256 |
| 48 | PDE2A | 0.9558 | 13.12 | 8.20E−06 |
| 49 | PDZD2 | 0.9139 | 34.56 | 0.004131 |
| 50 | PHLDB3 | 0.9494 | 39.41 | 1.43E−06 |
| 51 | PPARGC1B | 0.9028 | 10.56 | 0.0002482 |
| 52 | PTPRN2 | 0.9938 | 20.81 | 1.32E−06 |
| 53 | RASSF3 | 0.956 | 20.96 | 0.0003587 |
| 54 | RTN2 | 0.9919 | 21.95 | 2.10E−09 |
| 55 | RUNDC3A | 0.937 | 33 | 0.0001732 |
| 56 | RXRA | 0.9206 | 27.38 | 0.00304 |
| 57 | SAMD11 | 0.9792 | 13.05 | 0.0003039 |
| 58 | SLC38A2 | 0.9524 | 43.57 | 0.002501 |
| 59 | SLC38A3 | 0.941 | 15.42 | 6.02E−06 |
| 60 | SLC8A2 | 0.9156 | 15.67 | 0.0002196 |
| 61 | SPTBN4 | 0.9679 | 22.71 | 5.82E−07 |
| 62 | SRRM3 | 0.9579 | 47.41 | 5.42E−05 |
| 63 | STX10_A | 0.9208 | 51.95 | 0.00188 |
| 64 | STX10_B | 0.9614 | 39.5 | 0.0002594 |
| 65 | SYT5 | 0.9455 | 15.69 | 0.0006154 |
| 66 | SYT7 | 0.9011 | 7.743 | 0.0001346 |
| 67 | TGIF2 | 0.9425 | 27.97 | 2.25E−06 |
| 68 | TMC6_A | 0.9901 | 27.34 | 1.69E−07 |
| 69 | TMC6_B | 0.9557 | 14.43 | 3.50E−05 |
| 70 | TMEM145 | 0.9486 | 14.52 | 3.66E−05 |
| 71 | TNKS1BP1 | 0.9226 | 27.14 | 6.68E−05 |
| 72 | TSPO | 0.9667 | 9.424 | 3.38E−07 |

TABLE 2A

| | | Chromo- | DMR Start- |
|---|---|---|---|
| DMR | | some | End Positions |
| No. | Gene Annotation | No. | (GRCh37/hg19) |
| 73 | ABHD8 | 19 | 17403232-17403460 |
| 74 | ACAP1 | 17 | 7240013-7240106 |
| 75 | ARHGAP30 | 1 | 161039227-161039440 |
| 76 | AXIN1 | 16 | 375199-375316 |
| 77 | BCL9 | 1 | 147016991-147017127 |
| 78 | C1orf38 | 1 | 28195587-28195817 |
| 79 | C2orf85 | 2 | 242810333-242810434 |
| 80 | CACNA1C_B | 12 | 2800272-2800464 |
| 81 | CDK9 | 9 | 130545435-130545566 |
| 82 | CRMP1 | 4 | 5868034-5868199 |
| 83 | CSK | 15 | 75069555-75069761 |
| 84 | CTU2 | 16 | 88769741-88770116 |
| 85 | CUX1 | 7 | 101500189-101500515 |
| 86 | DEDD2 | 19 | 42703469-42703649 |
| 87 | ELMO1 | 7 | 37392953-37393050 |
| 88 | EPS15L1 | 19 | 16482561-16482712 |
| 89 | FAM129C | 19 | 17634027-17634213 |
| 90 | FAM78A | 9 | 134151363-134151474 |
| 91 | FERMT3 | 11 | 63974772-63974955 |
| 92 | FGF18 | 5 | 170878125-170878222 |
| 93 | FHAD1 | 1 | 15672512-15672643 |
| 94 | FNBP1 | 9 | 132650764-132651026 |
| 95 | GMFG | 19 | 39826176-39826273 |
| 96 | GNG7 | 19 | 2561967-2562206 |
| 97 | GRK6 | 5 | 176858662-176858774 |
| 98 | HIVEP3 | 1 | 42204698-42204867 |
| 99 | HMGA1 | 6 | 34203522-34203631 |
| 100 | HMHA1_A | 19 | 1069295-1069500 |
| 101 | HMHA1_B | 19 | 1074514-1074737 |
| 102 | HPCAL1 | 2 | 10471174-10471748 |
| 103 | IER2 | 19 | 13264692-13264807 |
| 104 | IL17C | 16 | 88700993-88701075 |
| 105 | INPP5D | 2 | 233925165-233925301 |
| 106 | KIAA0195 | 17 | 73483829-73483938 |
| 107 | KIAA0427 | 18 | 46361212-46361352 |
| 108 | LMTK2 | 7 | 97831890-97832023 |
| 109 | LOC100130872 | 4 | 1195670-1196131 |
| 110 | LOC100507463 | 6 | 32813441-32813592 |
| 111 | LOC285696 | 5 | 17130456-17130634 |
| 112 | MAD1L1 | 7 | 1980049-1980132 |
| 113 | MARK2 | 11 | 63637233-63637411 |
| 114 | MAX.chr1.16488894- | 1 | 16488894-16489075 |
| | 16489075 | | |
| 115 | MAX.chr1.210426160- | 1 | 210426160-210426264 |
| | 210426264 | | |
| 116 | MAX.chr1.225655507- | 1 | 225655507-225655620 |
| | 225655620 | | |
| 117 | MAX.chr11.68049738- | 11 | 68049738-68049894 |
| | 68049894 | | |
| 118 | MAX.chr12.12163358- | 12 | 12163358-12163631 |
| | 12163631 | | |
| 119 | MAX.chr14.102188698- | 14 | 102188698-102188818 |
| | 102188818 | | |
| 120 | MAX.chr14.107253099- | 14 | 107253099-107253355 |
| | 107253355 | | |
| 121 | MAX.chr15.31727007- | 15 | 31727007-31727144 |
| | 31727144 | | |
| 122 | MAX.chr15.70550976- | 15 | 70550976-70551130 |
| | 70551130 | | |
| 123 | MAX.chr16.11327016- | 16 | 11327016-11327312 |
| | 11327312 | | |
| 124 | MAX.chr16.50300428- | 16 | 50300428-50300651 |
| | 50300651 | | |
| 125 | MAX.chr16.50308404- | 16 | 50308404-50308570 |
| | 50308570 | | |
| 126 | MAX.chr17.74994454- | 17 | 74994454-74994572 |
| | 74994572 | | |
| 127 | MAX.chr17.76339840- | 17 | 76339840-76340086 |
| | 76340086 | | |
| 128 | MAX.chr2.10169502- | 2 | 10169502-10169736 |
| | 10169736 | | |
| 129 | MAX.chr2.235355101- | 2 | 235355101-235355212 |
| | 235355212 | | |
| 130 | MAX.chr20.56008091- | 20 | 56008091-56008227 |
| | 56008227 | | |
| 131 | MAX.chr3.187676577- | 3 | 187676577-187676668 |
| | 187676668 | | |
| 132 | MAX.chr4.4765181- | 4 | 4765181-4765330 |
| | 4765330 | | |
| 133 | MAX.chr5.53942200- | 5 | 53942200-53942315 |
| | 53942315 | | |
| 134 | MAX.chr6.159519777- | 6 | 159519777-159519949 |
| | 159519949 | | |
| 135 | MAX.chr6.170580966- | 6 | 170580966-170581132 |
| | 170581132 | | |
| 136 | MAX.chr6.20024141- | 6 | 20024141-20024570 |
| | 20024570 | | |
| 137 | MAX.chr6.24936094- | 6 | 24936094-24936246 |
| | 24936246 | | |
| 138 | MAX.chr7.391295- | 7 | 391295-391422 |
| | 391422 | | |
| 139 | MAX.chr8.142046288- | 8 | 142046288-142046398 |
| | 142046398 | | |
| 140 | MAX.chr8.142216497- | 8 | 142216497-142216631 |
| | 142216631 | | |
| 141 | MAX.chr8.144217550- | 8 | 144217550-144217700 |
| | 144217700 | | |
| 142 | MAX.chr8.145900710- | 8 | 145900710-145901246 |
| | 145901246 | | |
| 143 | MAX.chr8.80804237- | 8 | 80804237-80804308 |
| | 80804308 | | |
| 144 | MAX.chr9.87904996- | 9 | 87904996-87905372 |
| | 87905372 | | |
| 145 | MBP | 18 | 74818401-74818536 |
| 146 | MGAT1 | 5 | 180230498-180230723 |
| 147 | MIR200C | 12 | 7068171-7068303 |
| 148 | MOBKL2A | 19 | 2085442-2085612 |
| 149 | NBEAL2 | 3 | 47029453-47029597 |
| 150 | NCOR2_A | 12 | 124941846-124941955 |
| 151 | NCOR2_B | 12 | 124950687-124950803 |
| 152 | NELF | 9 | 140356296-140356348 |
| 153 | OSM_A | 22 | 30662000-30662103 |
| 154 | OSM_B | 22 | 30662697-30662807 |
| 155 | PARVG | 22 | 44577550-44577908 |
| 156 | PKN1 | 19 | 14551093-14551303 |
| 157 | PNMAL2 | 19 | 46996516-46996606 |
| 158 | PPP6R1 | 19 | 55765917-55766155 |
| 159 | PRIC285 | 20 | 62199539-62199703 |
| 160 | PRKAR1B | 7 | 644126-644374 |
| 161 | PTK2B | 8 | 27221308-27221453 |
| 162 | PTPRE | 10 | 129845667-129845938 |
| 163 | RAC2_A | 22 | 37626189-37626295 |
| 164 | RAC2_B | 22 | 37637570-37637727 |
| 165 | RAP1GAP2 | 17 | 2699553-2699729 |
| 166 | RASSF1 | 3 | 50378492-50378750 |
| 167 | RBM38 | 20 | 55964848-55965398 |
| 168 | RHOF | 12 | 122231058-122231184 |
| 169 | S1PR4_A | 19 | 3178378-3178781 |
| 170 | S1PR4_B | 19 | 3179828-3180413 |
| 171 | SDK2 | 17 | 71587461-71587557 |
| 172 | SEPTIN9_A | 17 | 75449912-75450101 |
| 173 | SEPTIN9_B | 17 | 75461597-75461735 |
| 174 | SH3BP2 | 4 | 2813867-2814151 |
| 175 | SHANK3 | 22 | 51110972-51111091 |
| 176 | SHISA5 | 3 | 48520645-48520772 |
| 177 | SHROOM1 | 5 | 132161293-132161522 |
| 178 | SKI | 1 | 2232144-2232470 |
| 179 | SNX20 | 16 | 50715181-50715339 |
| 180 | STAT5A | 17 | 40440733-40441156 |
| 181 | SUCLG2 | 3 | 67706348-67706568 |
| 182 | SUN2_A | 22 | 39148139-39148300 |
| 183 | SUN2_B | 22 | 39152758-39152893 |
| 184 | SUSD3 | 9 | 95821778-95821978 |
| 185 | TCF3 | 19 | 1650722-1650865 |
| 186 | TMC6_C | 17 | 76127199-76127566 |
| 187 | TMEM132E | 17 | 32964651-32964776 |
| 188 | TMEM163 | 2 | 135464600-135464735 |
| 189 | TNFRSF10C | 8 | 22961173-22961268 |
| 190 | TNFRSF25 | 1 | 6526055-6526198 |
| 191 | TRABD | 22 | 50629316-50629609 |
| 192 | TRAF3IP3 | 1 | 209943070-209943218 |
| 193 | UHRF1 | 19 | 4916882-4916984 |
| 194 | VAV1 | 19 | 6772930-6773075 |
| 195 | VILL | 3 | 38035405-38035508 |
| 196 | ZC3H12D | 6 | 149803439-149803687 |
| 197 | ZDHHC18 | 1 | 27160118-27160221 |
| 198 | ZFYVE28 | 4 | 2292779-2292933 |

TABLE 2B

| | | Area | | |
|---|---|---|---|---|
| DMR | | Under | Fold- | |
| No. | Gene Annotation | Curve | Change | p-value |
| 73 | ABHD8 | 0.9967 | 257.2 | 1.39E−08 |
| 74 | ACAP1 | 1 | 606.1 | 4.15E−06 |
| 75 | ARHGAP30 | 1 | 678.3 | 2.03E−11 |
| 76 | AXIN1 | 1 | 1146 | 5.59E−07 |
| 77 | BCL9 | 1 | 506.8 | 7.00E−06 |
| 78 | C1orf38 | 1 | 511.7 | 8.00E−07 |
| 79 | C2orf85 | 1 | 104.7 | 8.46E−08 |
| 80 | CACNA1C_B | 1 | 359 | 3.44E−05 |
| 81 | CDK9 | 1 | 6861 | 9.63E−05 |
| 82 | CRMP1 | 1 | 573.6 | 7.98E−11 |
| 83 | CSK | 1 | 164.7 | 1.25E−09 |
| 84 | CTU2 | 1 | 356.6 | 2.44E−18 |
| 85 | CUX1 | 1 | 1511 | 0.0007766 |
| 86 | DEDD2 | 1 | 686.5 | 5.75E−16 |
| 87 | ELMO1 | 1 | 2199 | 1.61E−08 |
| 88 | EPS15L1 | 1 | 100.3 | 3.76E−13 |
| 89 | FAM129C | 1 | 357 | 6.75E−10 |
| 90 | FAM78A | 1 | 2394 | 3.94E−09 |
| 91 | FERMT3 | 1 | 6902 | 0.002545 |
| 92 | FGF18 | 0.9782 | 103.7 | 0.0001005 |
| 93 | FHAD1 | 1 | 460.7 | 7.58E−15 |
| 94 | FNBP1 | 1 | 2139 | 2.36E−07 |
| 95 | GMFG | 1 | 445.8 | 2.66E−09 |
| 96 | GNG7 | 1 | 562.4 | 1.13E−09 |
| 97 | GRK6 | 1 | 1610 | 1.96E−10 |
| 98 | HIVEP3 | 1 | 197 | 5.04E−14 |
| 99 | HMGA1 | 1 | 418.8 | 0.0005092 |
| 100 | HMHA1_A | 1 | 619.7 | 0.0004255 |
| 101 | HMHA1_B | 1 | 212.2 | 3.57E−11 |
| 102 | HPCAL1 | 1 | 373.2 | 5.45E−12 |
| 103 | IER2 | 0.9806 | 508.5 | 0.009347 |
| 104 | IL17C | 1 | 828.8 | 1.33E−08 |
| 105 | INPP5D | 1 | 1836 | 8.39E−06 |
| 106 | KIAA0195 | 1 | 260.1 | 2.58E−12 |
| 107 | KIAA0427 | 0.9984 | 184.7 | 3.14E−11 |
| 108 | LMTK2 | 1 | 1776 | 5.52E−07 |
| 109 | LOC100130872 | 1 | 304.9 | 2.04E−14 |
| 110 | LOC100507463 | 1 | 1204 | 4.42E−14 |
| 111 | LOC285696 | 1 | 780.5 | 8.46E−12 |
| 112 | MAD1L1 | 1 | 1757 | 5.89E−12 |
| 113 | MARK2 | 1 | 246.7 | 3.90E−10 |
| 114 | MAX.chr1.16488894-16489075 | 1 | 258.5 | 5.63E−10 |
| 115 | MAX.chr1.210426160-210426264 | 0.9769 | 140.2 | 1.09E−05 |
| 116 | MAX.chr1.225655507-225655620 | 1 | 714.1 | 3.91E−06 |
| 117 | MAX.chr11.68049738-68049894 | 1 | 836.5 | 3.22E−07 |
| 118 | MAX.chr12.12163358-12163631 | 1 | 427.7 | 1.01E−06 |
| 119 | MAX.chr14.102188698-102188818 | 1 | 550 | 7.29E−06 |
| 120 | MAX.chr14.107253099-107253355 | 1 | 183.3 | 1.10E−05 |
| 121 | MAX.chr15.31727007-31727144 | 1 | 143.2 | 2.62E−13 |
| 122 | MAX.chr15.70550976-70551130 | 1 | 283.2 | 3.68E−16 |
| 123 | MAX.chr16.11327016-11327312 | 1 | 1448 | 8.67E−05 |
| 124 | MAX.chr16.50300428-50300651 | 1 | 263.9 | 1.08E−07 |
| 125 | MAX.chr16.50308404-50308570 | 1 | 1026 | 2.51E−11 |
| 126 | MAX.chr17.74994454-74994572 | 1 | 340.1 | 5.73E−23 |
| 127 | MAX.chr17.76339840-76340086 | 1 | 242.1 | 7.49E−18 |
| 128 | MAX.chr2.10169502-10169736 | 1 | 344.5 | 3.21E−11 |
| 129 | MAX.chr2.235355101-235355212 | 1 | 833.6 | 9.00E−14 |
| 130 | MAX.chr20.56008091-56008227 | 1 | 1437 | 8.70E−10 |
| 131 | MAX.chr3.187676577-187676668 | 1 | 605.6 | 5.73E−13 |
| 132 | MAX.chr4.4765181-4765330 | 0.9984 | 121.1 | 9.86E−11 |
| 133 | MAX.chr5.53942200-53942315 | 1 | 281.6 | 1.71E−15 |
| 134 | MAX.chr6.159519777-159519949 | 1 | 140.7 | 6.66E−11 |
| 135 | MAX.chr6.170580966-170581132 | 1 | 249.1 | 1.20E−12 |
| 136 | MAX.chr6.20024141-20024570 | 1 | 324.6 | 9.02E−21 |
| 137 | MAX.chr6.24936094-24936246 | 1 | 450.6 | 6.59E−07 |
| 138 | MAX.chr7.391295-391422 | 0.9783 | 286.9 | 9.61E−11 |
| 139 | MAX.chr8.142046288-142046398 | 1 | 1012 | 5.66E−06 |
| 140 | MAX.chr8.142216497-142216631 | 1 | 197.9 | 7.42E−07 |
| 141 | MAX.chr8.144217550-144217700 | 0.9581 | 146.4 | 7.12E−17 |
| 142 | MAX.chr8.145900710-145901246 | 1 | 596.4 | 1.23E−30 |
| 143 | MAX.chr8.80804237-80804308 | 1 | 296.9 | 1.15E−05 |
| 144 | MAX.chr9.87904996-87905372 | 1 | 356.7 | 5.38E−18 |
| 145 | MBP | 1 | 486.8 | 3.78E−15 |
| 146 | MGAT1 | 1 | 1066 | 4.04E−10 |
| 147 | MIR200C | 1 | 769.4 | 5.84E−11 |
| 148 | MOBKL2A | 1 | 3343 | 0.0004202 |
| 149 | NBEAL2 | 1 | 2617 | 1.81E−07 |
| 150 | NCOR2_A | 1 | 304.9 | 5.69E−11 |
| 151 | NCOR2_B | 1 | 457.2 | 4.35E−11 |
| 152 | NELF | 1 | 905.7 | 3.88E−05 |
| 153 | OSM_A | 1 | 1317 | 1.07E−10 |
| 154 | OSM_B | 1 | 897.5 | 9.33E−09 |
| 155 | PARVG | 1 | 412.3 | 4.08E−06 |
| 156 | PKN1 | 1 | 354.8 | 4.63E−14 |
| 157 | PNMAL2 | 0.9984 | 154.4 | 1.24E−08 |
| 158 | PPP6R1 | 1 | 544.1 | 1.65E−06 |
| 159 | PRIC285 | 1 | 398.8 | 3.43E−21 |
| 160 | PRKAR1B | 1 | 517.4 | 1.87E−13 |
| 161 | PTK2B | 1 | 547.3 | 1.03E−10 |
| 162 | PTPRE | 1 | 1138 | 1.45E−05 |
| 163 | RAC2_A | 1 | 1953 | 0.0001195 |
| 164 | RAC2_B | 1 | 184.3 | 1.45E−11 |
| 165 | RAP1GAP2 | 1 | 1875 | 3.91E−05 |
| 166 | RASSF1 | 1 | 353.5 | 5.28E−13 |
| 167 | RBM38 | 1 | 164.3 | 5.90E−15 |
| 168 | RHOF | 1 | 456.5 | 4.76E−07 |
| 169 | S1PR4_A | 1 | 3268 | 5.18E−09 |
| 170 | S1PR4_B | 1 | 1640 | 9.87E−14 |
| 171 | SDK2 | 1 | 490.5 | 7.27E−09 |
| 172 | SEPTIN9_A | 1 | 358.4 | 2.80E−10 |
| 173 | SEPTIN9_B | 1 | 197.9 | 9.73E−12 |
| 174 | SH3BP2 | 1 | 710.9 | 2.18E−06 |
| 175 | SHANK3 | 1 | 123.4 | 1.56E−18 |
| 176 | SHISA5 | 1 | 523.2 | 2.96E−13 |
| 177 | SHROOM1 | 1 | 368.2 | 3.10E−25 |
| 178 | SKI | 1 | 198.5 | 6.16E−18 |
| 179 | SNX20 | 1 | 965.2 | 2.45E−08 |
| 180 | STAT5A | 1 | 462.7 | 4.41E−12 |
| 181 | SUCLG2 | 1 | 2193 | 7.49E−19 |
| 182 | SUN2_A | 1 | 4978 | 8.42E−06 |
| 183 | SUN2_B | 1 | 285.7 | 5.54E−09 |
| 184 | SUSD3 | 1 | 646.6 | 5.09E−21 |
| 185 | TCF3 | 1 | 108.9 | 8.36E−09 |
| 186 | TMC6_C | 1 | 1019 | 3.82E−22 |
| 187 | TMEM132E | 1 | 185.3 | 4.91E−09 |
| 188 | TMEM163 | 1 | 584.1 | 8.19E−06 |
| 189 | TNFRSF10C | 1 | 579.4 | 1.64E−08 |
| 190 | TNFRSF25 | 1 | 145.2 | 4.19E−09 |
| 191 | TRABD | 1 | 204.3 | 6.93E−08 |
| 192 | TRAF3IP3 | 1 | 239.7 | 1.32E−10 |
| 193 | UHRF1 | 1 | 884.8 | 4.78E−14 |
| 194 | VAV1 | 1 | 610.6 | 2.89E−10 |
| 195 | VILL | 1 | 131.6 | 1.28E−09 |
| 196 | ZC3H12D | 1 | 165.9 | 4.91E−13 |
| 197 | ZDHHC18 | 1 | 1722 | 6.65E−13 |
| 198 | ZFYVE28 | 1 | 388.5 | 2.58E−20 |

TABLE 3

| | | | Forward | | Reverse |
|---|---|---|---|---|---|
| | | Seq | Primer | Seq | 5Primer |
| DMR | Gene | ID | 5′-3′ | ID | 5′-3′ |
| No. | Annotation | No. | Sequence | No. | Sequence |
| 3 | ANXA2 | 1 | AGGATTGTTTGTT | 2 | ACTATACTCCGCT |
| | | | ACGAGGTCGCGT | | TCTCTCCGCGCC |
| 6 | CACNA1C_A | 3 | GGTCGCGGCGTTT | 4 | AACTCATCCTCCC |
| | | | GTTTAGAGGC | | TCCCGAAACGTC |
| 8 | CDHR2 | 5 | CGAGTTTAGTGGT | 6 | TACGAAATCCGAA |
| | | | TTTTAGGTAACGG | | AAAAATCCGTA |
| 14 | FBXL16_B | 7 | TCGAGGCGGTAGT | 8 | AATCTAAAAAACG |
| | | | ATTAGGTTTACG | | AAAATCCCCGCT |
| 17 | GP1BB_A | 9 | TTTATTTTGTAGC | 10 | CTAAACTATTTCT |
| | | | GGGAGGCGTAGGC | | AACCAAACCGCA |
| 19 | GP1BB_C | 11 | TAGTAGAGCGGGT | 12 | CGCCTACTACCCT |
| | | | CGGGAGCGTAAGC | | ATCTAACCGAAAA |
| | | | | | CGAAC |
| 20 | HCN2 | 13 | TTGGGAAGTTTGC | 14 | AAAATCCGTAAAA |
| | | | GGTTTTTTCGTT | | ACTATCCTAAAAC |
| | | | | | GCCC |
| 21 | HPCAL1 | 15 | AGGATGTAGTTTA | 16 | GAAAAACGCCAAT |
| | | | GTTCGTGGAGTTC | | TTTACGCCGTAA |
| | | | GAG | | |
| 29 | LOC100129726 | 17 | GTGTAATTTGGGT | 18 | CGTACCTTTAACA |
| | | | CGCGGTTTTCGC | | CGCGCGATACGTT |
| 38 | MAX.chr17.7778 | 19 | TTAGGGTCGGGAA | 20 | GAAACCGAACTCG |
| | 8758-77788971 | | AGGATTTTTTATC | | AAATCCACGCG |
| | | | GT | | |
| 40 | MAX.chr19.2478 | 21 | GATTTTGGGTTGC | 22 | AAACACATATAAA |
| | 419-2478656 | | GGTGGTCGT | | AACATTTCAACGA |
| | | | | | A |
| 49 | PDZD2 | 23 | AGTTATAGTTTCG | 24 | CCGAAAAACGAAA |
| | | | GAGGCGCGGAGC | | AAAACAAACGCT |
| 52 | PTPRN2 | 25 | CGGGTTATAGTTA | 26 | ACTCTCGCCAACT |
| | | | TAGGTTGGGGTAT | | CCGCGAA |
| | | | TTCGG | | |
| 53 | RASSF3 | 27 | GCGGTTTTTTGGT | 28 | ATAAACGTAACCG |
| | | | ATTAGGAGTCGT | | AATTAACCCGAC |
| 54 | RTN2 | 29 | TTTTATTGAAGTG | 30 | TCCGAATAAAAAA |
| | | | GGTAAAATTTTCG | | CTAAAAACACCGC |
| | | | AG | | TA |
| 55 | RUNDC3A | 31 | TTAGGGGTTAGGG | 32 | CGCGAAAAACGAA |
| | | | TAGGTCGTGCGT | | AACTAAAAAACGT |
| | | | | | A |
| 56 | RXRA | 33 | CGTTTGTTTAGGA | 34 | GCCGTCTCGAACG |
| | | | AGGTTGGGTTTGG | | ACTAAAATTCGAA |
| | | | C | | |
| 58 | SLC38A2 | 35 | ACGGTTATGGAAA | 36 | CCAAACGACCTTA |
| | | | TTGGATTAGCGG | | AAAACGCCGAA |
| 61 | SPTBN4 | 37 | TCGGATTGCGGGA | 38 | CACGTCGAAATAA |
| | | | GGTTGTC | | TACTACTCCACCT |
| | | | | | AAAAAACG |
| 62 | SRRM3 | 39 | TCGTTTTAGCGGA | 40 | GTACGTACATCGA |
| | | | GACGCGG | | ACGAACTATACGC |
| | | | | | CGAAC |
| 64 | STX10_B | 41 | TTATAGTATTAGG | 42 | AACGATTCCTCGA |
| | | | TGGAGTTGAGCGG | | AAAAAATACGAA |
| 68 | TMC6_A | 43 | GTTTTGTTTGGGG | 44 | AAACAAAAACCGA |
| | | | TTTTGGGTTCGG | | CAAAACTCGCT |
| 72 | TSPO | 45 | TTTAGGTGCGTTG | 46 | AAACAAATCCCAA |
| | | | TAGTTTAGACGG | | AAACTACTCGAC |
| 85 | CUX1 (v1) | 47 | GGAGAGGATTTGA | 48 | CTCTAAAATCCTA |
| | | | AGGGTTTCGT | | CCCAACTCCGAT |
| 85 | CUX1 (v2) | 49 | CGTAGGTTTAAAA | 50 | CCGATTCCTATTT |
| | | | GTGGTTCGCGGC | | CTATTAAAACGAA |
| 90 | FAM78A | 51 | GGGTGTTCGGTAG | 52 | ATAAAAACCTCCA |
| | | | CGGAGTATTACGT | | TCGACCCCGTCC |
| | | | T | | |
| 94 | FNBP1 | 53 | GCGTGATTGATGG | 54 | ATAAACTTCCGAT |
| | | | GTGTATTACGT | | CCCTACAACGAA |
| 103 | IER2 | 55 | GTCGAATCGTCGG | 56 | TCTCCACGATTTT |
| | | | TTCGAGGGC | | CGCGAACGCT |
| 148 | MOBKL2A | 57 | ATTTTGTTATTGT | 58 | CACTCAAAACTTA |
| | | | TTCGGGGATCGT | | TCTCTCAAACGCC |
| 157 | PNMAL2 | 59 | GAGGAAAGAGAAG | 60 | CCCAATCCTAATC |
| | | | TGGGCGTTCGA | | CACTTAACGCGTC |
| 169 | S1PR4_A | 61 | GGTTGGAAAGGGG | 62 | GAAAACCCGCAAA |
| | | | TGGTTTATTTCGA | | AAACCCCGAA |
| 26 | LGALS3 | 63 | GCGTTACGGAATT | 64 | CGACGAAAAAAAC |
| | | | TAACGGTGGTAGC | | GCGAACACTAAAA |
| | | | G | | AACG |
| 45 | MYO15B | 65 | TTTCGAGGATAGT | 66 | ATTATCGCTCGCG |
| | | | TCGCGGGTTTTTC | | TCCTTAACCGAC |

TABLE 4

| | | AUC PNET | |
|---|---|---|---|
| | | Tissue vs | |
| | | Benign | |
| | | Pancreatic | AUC PNET |
| DMR | | Tissue | Tissue vs |
| No. | Gene Annotation | (Normal) | Buffy Coat |
| 3 | ANXA2 | 0.81119 | 1 |
| 6 | CACNA1C_A | 0.86888 | 0.99621 |
| 8 | CDHR2 | 0.96329 | 0.99621 |
| 14 | FBXL16_B | 0.92657 | 0.98106 |
| 17 | GP1BB_A | 0.88986 | 1 |
| 19 | GP1BB_C | 0.95629 | 1 |
| 20 | HCN2 | 0.96154 | 0.97917 |
| 21 | HPCAL1 | 0.8951 | 0.97159 |
| 26 | LGALS3 | 0.80944 | 0.87879 |
| 29 | LOC100129726 | 0.76049 | 0.91098 |
| 38 | MAX.chr17.77788758- | 0.87325 | 0.9536 |
| | 77788971 | | |
| 40 | MAX.chr19.2478419- | 0.84091 | 0.99242 |
| | 2478656 | | |
| 45 | MYO15B | 0.88287 | 0.73011 |
| 49 | PDZD2 | 0.89336 | 0.94318 |
| 52 | PTPRN2 | 0.95105 | 0.97727 |
| 53 | RASSF3 | 0.87762 | 0.93939 |
| 54 | RTN2 | 0.94056 | 1 |
| 55 | RUNDC3A | 0.90909 | 0.95265 |
| 56 | RXRA | 0.72552 | 0.94886 |
| 58 | SLC38A2 | 0.88287 | 0.99811 |
| 61 | SPTBN4 | 0.98427 | 1 |
| 62 | SRRM3 | 0.95717 | 0.98485 |
| 64 | STX10_B | 0.95105 | 1 |
| 68 | TMC6_A | 0.96503 | 1 |
| 72 | TSPO | 0.87238 | 0.97727 |
| 85 | CUX1 (with primer SEQ | 0.81643 | 1 |
| | ID Nos: 47 and 48) | | |
| 85 | CUX1 (with primer SEQ | 0.78671 | 1 |
| | ID Nos: 49 and 50) | | |
| 90 | FAM78A | 0.63112 | 1 |
| 94 | FNBP1 | 0.50699 | 1 |
| 103 | IER2 | 0.8007 | 0.93371 |
| 148 | MOBKL2A | 0.56643 | 1 |
| 157 | PNMAL2 | 0.52273 | 1 |
| 169 | S1PR4_A | 0.54545 | 1 |

TABLE 5A

| | | | Cystic | Solid | | |
|---|---|---|---|---|---|---|
| | | Pancreas | PNET | PNET | | |
| | | Neuroendocrine | Tissue | Tissue | Metastatic | PNET |
| | | Tumor | Vs | Vs | PNET | Tissue Vs |
| | | (PNET) Tissue | Normal | Normal | Tissue Vs | Metastatic |
| | | Vs Normal | Tissue | Tissue | Normal | PNET |
| | | Pancreas | AUC | AUC | Tissue | Tissue |
| DMR | | Tissue | (95% | (95% | AUC (95% | AUC (95% |
| No. | Gene Annotation | AUC (95% CI) | CI) | CI) | CI) | CI) |
| 62 | SRRM3 | 0.96 (0.93-1) | 0.96 | 0.97 | 0.94 (0.85- | 0.49 (0.34- |
| | | | (0.88- | (0.92- | 1.02) | 0.63) |
| | | | 1.03) | 1.01) | | |
| 20 | HCN2 | 0.96 (0.92-1) | 0.91 | 0.97 | 0.89 (0.78- | 0.4 (0.25- |
| | | | (0.81- | (0.93- | 1) | 0.54) |
| | | | 1.02) | 1.01) | | |
| 61 | SPTBN4 | 0.96 (0.92-1) | 0.86 | 0.99 | 0.99 (0.97- | 0.39 (0.27- |
| | | | (0.71- | (0.97- | 1.01) | 0.52) |
| | | | 1.01) | 1) | | |
| 68 | TMC6_A | 0.95 (0.9-1) | 0.94 | 0.95 | 0.89 (0.77- | 0.37 (0.23- |
| | | | (0.84- | (0.9-1) | 1) | 0.5) |
| | | | 1.04) | | | |
| 19 | GP1BB_C | 0.93 (0.88- | 0.93 | 0.93 | 0.76 (0.61- | 0.43 (0.27- |
| | | 0.98) | (0.84- | (0.87- | 0.9) | 0.6) |
| | | | 1.03) | 0.99) | | |
| 17 | GP1BB_A | 0.93 (0.87- | 0.96 | 0.91 | 0.78 (0.64- | 0.34 (0.2- |
| | | 0.98) | (0.9- | (0.85- | 0.91) | 0.48) |
| | | | 1.02) | 0.98) | | |
| 64 | STX10_B | 0.92 (0.87- | 0.88 | 0.93 | 0.86 (0.75- | 0.36 (0.23- |
| | | 0.98) | (0.76- | (0.88- | 0.97) | 0.49) |
| | | | 1.01) | 0.99) | | |
| 6 | CACNA1C_A | 0.92 (0.86- | 0.87 | 0.93 | 0.83 (0.7- | 0.42 (0.28- |
| | | 0.98) | (0.74- | (0.88- | 0.96) | 0.56) |
| | | | 1) | 0.99) | | |
| 8 | CDHR2 | 0.92 (0.86- | 0.88 | 0.93 | 0.83 (0.7- | 0.44 (0.3- |
| | | 0.98) | (0.74- | (0.86- | 0.96) | 0.58) |
| | | | 1.02) | 0.99) | | |
| 52 | PTPRN2 | 0.91 (0.85- | 0.9 | 0.92 | 0.93 (0.84- | 0.53 (0.39- |
| | | 0.98) | (0.76- | (0.84- | 1.03) | 0.67) |
| | | | 1.04) | 0.99) | | |
| 38 | MAX.chr17. | 0.91 (0.85- | 0.92 | 0.9 | 0.92 (0.85- | 0.58 (0.44- |
| | 77788758.77788971 | 0.97) | (0.83- | (0.83- | 1) | 0.72) |
| | | | 1.01) | 0.97) | | |
| 14 | FBXL16_B | 0.9 (0.84-0.97) | 0.89 | 0.91 | 0.77 (0.63- | 0.38 (0.25- |
| | | | (0.75- | (0.83- | 0.92) | 0.51) |
| | | | 1.04) | 0.98) | | |
| 54 | RTN2 | 0.9 (0.84-0.96) | 0.88 | 0.91 | 0.92 (0.84- | 0.56 (0.43- |
| | | | (0.74- | (0.84- | 1.01) | 0.69) |
| | | | 1.02) | 0.98) | | |
| 21 | HPCAL1 | 0.86 (0.79- | 0.95 | 0.84 | 0.89 (0.8- | 0.49 (0.36- |
| | | 0.94) | (0.87- | (0.74- | 0.99) | 0.63) |
| | | | 1.02) | 0.93) | | |
| 53 | RASSF3 | 0.85 (0.77- | 0.9 | 0.84 | 0.82 (0.68- | 0.42 (0.28- |
| | | 0.93) | (0.77- | (0.74- | 0.96) | 0.55) |
| | | | 1.03) | 0.93) | | |
| 72 | TSPO | 0.85 (0.77- | 0.85 | 0.85 | 0.84 (0.72- | 0.49 (0.34- |
| | | 0.93) | (0.7- | (0.76- | 0.97) | 0.63) |
| | | | 0.99) | 0.94) | | |
| 55 | RUNDC3A | 0.81 (0.72-0.9) | 0.9 | 0.78 | 0.72 (0.56- | 0.45 (0.3- |
| | | | (0.76- | (0.67- | 0.88) | 0.59) |
| | | | 1.04) | 0.89) | | |
| 58 | SLC38A2 | 0.78 (0.69- | 0.83 | 0.77 | 0.79 (0.64- | 0.47 (0.34- |
| | | 0.88) | (0.67- | (0.66- | 0.93) | 0.6) |
| | | | 0.99) | 0.88) | | |
| 40 | MAX.chr19. | 0.77 (0.67- | 0.75 | 0.77 | 0.68 (0.51- | 0.43 (0.3- |
| | 2478419.2478656 | 0.86) | (0.56- | (0.67- | 0.84) | 0.56) |
| | | | 0.94) | 0.88) | | |
| 49 | PDZD2 | 0.76 (0.67- | 0.69 | 0.79 | 0.66 (0.5- | 0.37 (0.25- |
| | | 0.86) | (0.49- | (0.69- | 0.81) | 0.5) |
| | | | 0.89) | 0.89) | | |
| 29 | LOC100129726 | 0.75 (0.66- | 0.83 | 0.72 | 0.87 (0.76- | 0.58 (0.45- |
| | | 0.85) | (0.68- | (0.61- | 0.99) | 0.71) |
| | | | 0.99) | 0.84) | | |
| 85 | CUX1 (with primer SEQ ID | 0.67 (0.56- | 0.61 | 0.69 | 0.48 (0.31- | 0.38 (0.25- |
| | Nos: 47 and 48) | 0.77) | (0.39- | (0.56- | 0.66) | 0.51) |
| | | | 0.83) | 0.81) | | |
| 3 | ANXA2 | 0.66 (0.55- | 0.57 | 0.69 | 0.73 (0.57- | 0.56 (0.42- |
| | | 0.77) | (0.37- | (0.57- | 0.88) | 0.69) |
| | | | 0.77) | 0.81) | | |
| 56 | RXRA | 0.62 (0.51- | 0.57 | 0.64 | 0.57 (0.39- | 0.46 (0.33- |
| | | 0.73) | (0.35- | (0.52- | 0.74) | 0.6) |
| | | | 0.78) | 0.77) | | |
| 85 | CUX1 (with primer SEQ ID | 0.62 (0.51- | 0.56 | 0.64 | 0.44 (0.26- | 0.39 (0.26- |
| | Nos: 49 and 50) | 0.73) | (0.33- | (0.51- | 0.62) | 0.51) |
| | | | 0.79) | 0.76) | | |
| 169 | S1PR4_A | 0.6 (0.49-0.71) | 0.73 | 0.56 | 0.43 (0.25- | 0.38 (0.25- |
| | | | (0.54- | (0.43- | 0.6) | 0.51) |
| | | | 0.92) | 0.69) | | |
| 94 | FNBP1 | 0.51 (0.39- | 0.68 | 0.45 | 0.29 (0.13- | 0.36 (0.23- |
| | | 0.63) | (0.5- | (0.32- | 0.45) | 0.49) |
| | | | 0.87) | 0.58) | | |
| 90 | FAM78A | 0.5 (0.36-0.64) | 0.64 | 0.45 | 0.4 (0.24- | 0.39 (0.24- |
| | | | (0.47- | (0.3- | 0.56) | 0.54) |
| | | | 0.82) | 0.6) | | |
| 103 | IER2 | 0.49 (0.37-0.6) | 0.42 | 0.51 | 0.57 (0.4- | 0.59 (0.47- |
| | | | (0.21- | (0.38- | 0.74) | 0.71) |
| | | | 0.64) | 0.64) | | |
| 157 | PNMAL2 | 0.37 (0.24- | 0.34 | 0.39 | 0.36 (0.21- | 0.51 (0.38- |
| | | 0.51) | (0.17- | (0.25- | 0.52) | 0.64) |
| | | | 0.51) | 0.53) | | |
| 148 | MOBKL2A | 0.33 (0.21- | 0.42 | 0.3 | 0.22 (0.08- | 0.39 (0.26- |
| | | 0.46) | (0.24- | (0.18- | 0.35) | 0.51) |
| | | | 0.61) | 0.43) | | |

TABLE 5B

| | | Small Bowel | Lung NET |
|---|---|---|---|
| | | Neuroendocrine | Tissue Vs |
| | | Tumor (NET) | PNET |
| | | Tissue Vs | Tissue |
| DMR | | PNET Tissue | AUC |
| No. | Gene Annotation | AUC (95% CI) | (95% CI) |
| 62 | SRRM3 | 0.37 (0.26-0.49) | 0.44 (0.32-0.57) |
| 20 | HCN2 | 0.35 (0.23-0.47) | 0.71 (0.6-0.81) |
| 61 | SPTBN4 | 0.45 (0.33-0.56) | 0.62 (0.5-0.74) |
| 68 | TMC6_A | 0.27 (0.17-0.38) | 0.67 (0.56-0.78) |
| 19 | GP1BB_C | 0.32 (0.21-0.43) | 0.78 (0.69-0.88) |
| 17 | GP1BB_A | 0.38 (0.27-0.5) | 0.68 (0.57-0.79) |
| 64 | STX10_B | 0.4 (0.29-0.51) | 0.33 (0.22-0.45) |
| 6 | CACNA1C_A | 0.3 (0.2-0.4) | 0.26 (0.15-0.37) |
| 8 | CDHR2 | 0.33 (0.23-0.44) | 0.65 (0.54-0.76) |
| 52 | PTPRN2 | 0.38 (0.27-0.49) | 0.56 (0.44-0.68) |
| 38 | MAX.chr17.77788758. | 0.17 (0.08-0.25) | 0.49 (0.38-0.6) |
| | 77788971 | | |
| 14 | FBXL16_B | 0.61 (0.5-0.72) | 0.77 (0.66-0.87) |
| 54 | RTN2 | 0.4 (0.29-0.51) | 0.58 (0.46-0.71) |
| 21 | HPCAL1 | 0.14 (0.07-0.22) | 0.5 (0.39-0.62) |
| 53 | RASSF3 | 0.25 (0.15-0.35) | 0.4 (0.29-0.52) |
| 72 | TSPO | 0.36 (0.25-0.47) | 0.65 (0.54-0.77) |
| 55 | RUNDC3A | 0.46 (0.34-0.57) | 0.43 (0.31-0.55) |
| 58 | SLC38A2 | 0.18 (0.1-0.27) | 0.41 (0.3-0.52) |
| 40 | MAX.chr19.2478419. | 0.2 (0.11-0.29) | 0.35 (0.23-0.46) |
| | 2478656 | | |
| 49 | PDZD2 | 0.17 (0.09-0.25) | 0.14 (0.07-0.21) |
| 29 | LOC100129726v1 | 0.22 (0.12-0.31) | 0.62 (0.5-0.75) |
| 85 | CUX1 (with primer | 0.39 (0.28-0.5) | 0.38 (0.26-0.49) |
| | SEQ ID Nos: 47 and 48) | | |
| 3 | ANXA2 | 0.31 (0.2-0.42) | 0.57 (0.45-0.68) |
| 56 | RXRA | 0.23 (0.13-0.32) | 0.39 (0.27-0.5) |
| 85 | CUX1 (with primer | 0.42 (0.3-0.53) | 0.41 (0.29-0.53) |
| | SEQ ID Nos: 49 and 50) | | |
| 169 | S1PR4_A | 0.52 (0.4-0.64) | 0.61 (0.5-0.73) |
| 94 | FNBP1 | 0.59 (0.48-0.71) | 0.49 (0.37-0.61) |
| 90 | FAM78A | 0.48 (0.36-0.6) | 0.71 (0.6-0.81) |
| 103 | IER2 | 0.66 (0.55-0.77) | 0.49 (0.38-0.61) |
| 157 | PNMAL2 | 0.26 (0.16-0.36) | 0.61 (0.49-0.72) |
| 148 | MOBKL2A | 0.63 (0.52-0.74) | 0.71 (0.6-0.82) |

TABLE 5C

| | | PNET | Meta- | | Small |
|---|---|---|---|---|---|
| | | Tissue | static | Lung | Bowel |
| | | Vs | PNET | NET | NET |
| | | Buffy | Tissue | Tissue | Tissue |
| | | Coat | Vs Buffy | Vs Buffy | Vs Buffy |
| | | AUC | Coat | Coat | Coat |
| DMR | | (95% | AUC | AUC | AUC |
| No. | Gene Annotation | CI) | (95% CI) | (95% CI) | (95% CI) |
| 62 | SRRM3 | 1 (1-1) | 1 (1-1) | 0.99 | 1 |
| | | | | (0.99-1) | (0.99-1) |
| 20 | HCN2 | 1 (1-1) | 0.99 | 1 (1-1) | 1 (1-1) |
| | | | (0.97- | | |
| | | | 1.01) | | |
| 61 | SPTBN4 | 0.99 | 0.99 | 0.99 | 0.99 |
| | | (0.98-1) | (0.97- | (0.98-1) | (0.98-1) |
| | | | 1.01) | | |
| 68 | TMC6_A | 1 (1-1) | 0.99 | 1 (1-1) | 0.98 |
| | | | (0.97- | | (0.93- |
| | | | 1.01) | | 1.02) |
| 19 | GP1BB_C | 1 (1-1) | 0.99 | 1 (1-1) | 1 |
| | | | (0.98- | | (0.99-1) |
| | | | 1.01) | | |
| 17 | GP1BB_A | 1 (1-1) | 1 (1-1) | 1 (1-1) | 1 |
| | | | | | (0.99-1) |
| 64 | STX10_B | 1 (1-1) | 1 | 0.99 | 1 (1-1) |
| | | | (0.99- | (0.98- | |
| | | | 1.01) | 1.01) | |
| 6 | CACNA1C_A | 1 (1-1) | 1 | 0.98 | 1 (1-1) |
| | | | (0.99-1) | (0.94- | |
| | | | | 1.02) | |
| 8 | CDHR2 | 1 (1-1) | 1 (1-1) | 1 (1-1) | 1 (1-1) |
| 52 | PTPRN2 | 0.93 | 0.94 | 0.94 | 0.97 |
| | | (0.88- | (0.87- | (0.89-1) | (0.93- |
| | | 0.98) | 1.01) | | 1.01) |
| 38 | MAX.chr17. | 0.96 | 0.97 | 0.99 | 0.84 |
| | 77788758. | (0.93- | (0.93-1) | (0.97- | (0.74- |
| | 77788971 | 0.99) | | 1.01) | 0.94) |
| 14 | FBXL16_B | 1 (1-1) | 1 | 1 (1-1) | 1 (1-1) |
| | | | (0.99-1) | | |
| 54 | RTN2 | 0.97 | 0.99 | 0.96 | 1 |
| | | (0.95-1) | (0.96- | (0.93-1) | (0.99-1) |
| | | | 1.01) | | |
| 21 | HPCAL1 | 0.84 | 0.84 | 0.87 | 0.56 |
| | | (0.76- | (0.75- | (0.8- | (0.41- |
| | | 0.91) | 0.94) | 0.95) | 0.7) |
| 53 | RASSF3 | 0.91 | 0.9- | 0.95 | 0.77 |
| | | (0.85- | (0.81- | (0.89-1) | (0.66- |
| | | 0.97) | 0.99) | | 0.89) |
| 72 | TSPO | 0.92 | 0.92 | 0.95 | 0.93 |
| | | (0.86- | (0.85- | (0.9- | (0.87- |
| | | 0.97) | 0.99) | 1.01) | 0.98) |
| 55 | RUNDC3A | 0.99 | 0.99 | 1 | 1 |
| | | (0.98-1) | (0.98-1) | (0.99-1) | (0.99-1) |
| 58 | SLC38A2 | 1 | 1 | 1 | 0.98 |
| | | (0.99-1) | (0.99-1) | (0.99-1) | (0.96- |
| | | | | | 1.01) |
| 40 | MAX.chr19. | 0.86 | 0.8 | 0.67 | 0.5 |
| | 2478419. | (0.79- | (0.66- | (0.53- | (0.36- |
| | 2478656 | 0.94) | 0.94) | 0.81) | 0.65) |
| 49 | PDZD2 | 0.83 | 0.75 | 0.43 | 0.51 |
| | | (0.75- | (0.62- | (0.29- | (0.37- |
| | | 0.91) | 0.88) | 0.56) | 0.64) |
| 29 | LOC100129726v1 | 0.99 | 1 | 1 | 1 |
| | | (0.98-1) | (0.99-1) | (0.99-1) | (0.99-1) |
| 85 | CUX1 (with | 1 (1-1) | 1 (1-1) | 1 (1-1) | 1 (1-1) |
| | primer SEQ ID | | | | |
| | Nos: 47 and 48) | | | | |
| 3 | ANXA2 | 1 (1-1) | 1 | 1 (1-1) | 1 (1-1) |
| | | | (0.99-1) | | |
| 56 | RXRA | 0.91 | 0.9 | 0.86 | 0.8 |
| | | (0.85- | (0.82- | (0.78- | (0.68- |
| | | 0.97) | 0.97) | 0.95) | 0.92) |
| 85 | CUX1 (with | 1 (1-1) | 1 (1-1) | 1 (1-1) | 1 (1-1) |
| | primer SEQ ID | | | | |
| | Nos: 49 and 50) | | | | |
| 169 | S1PR4_A | 1 (1-1) | 1 (1-1) | 1 (1-1) | 1 (1-1) |
| 94 | FNBP1 | 1 (1-1) | 1(1-1) | 1 (1-1) | 1 (1-1) |
| 90 | FAM78A | 1 (1-1) | 1 (1-1) | 1 (1-1) | 1 (1-1) |
| 103 | IER2 | 1 (1-1) | 1 (1-1) | 1 | 1 (1-1) |
| | | | | (0.99-1) | |
| 157 | PNMAL2 | 0.98 | 0.98 | 0.99 | 0.96 |
| | | (0.96- | (0.96- | (0.97- | (0.9- |
| | | 1.01) | 1.01) | 1.01) | 1.01) |
| 148 | MOBKL2A | 1 (1-1) | 1 (1-1) | 1 (1-1) | 1 (1-1) |

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

We claim:

1. A method, comprising:
measuring a methylation level for one or more genes in a biological sample of a human individual through
treating genomic DNA in the biological sample with a reagent that modifies DNA in a methylation-specific manner;
amplifying the treated genomic DNA using a set of primers for the selected one or more genes; and
determining the methylation level of the one or more genes by polymerase chain reaction, nucleic acid sequencing, mass spectrometry, methylation-specific nuclease, mass-based separation, and target capture;
wherein the one or more genes is selected from Table 1A and 2A.

2. The method of claim 1, wherein the one or more genes are selected from ANXA2, CACNA1C_A, CDHR2, FBXL16_B, GP1BB_A, GP1BB_C, HCN2, HPCAL1, LOC100129726, MAX.chr17.77788758-77788971, PDZD2, PTPRN2, RASSF3, RTN2, RUNDC3A, RXRA, SLC38A2, SPTBN4, SRRM3, STX10_B, TMC6_A, TSPO, CUX1, FAM78A, FNBP1, IER2, MOBKL2A, PNMAL2, S1PR4_A, LGALS3, and MYO15B.

3. The method of claim 1, wherein the one or more genes are selected from SRRM3, HCN2, SPTBN4, TMC6_A, GP1BB_C, GP1BB_A, STX10_B, CACNA1C_A, CDHR2, PTPRN2, MAX.chr17.77788758.77788971, FBXL16_B, RTN2, HPCAL1, RASSF3, TSPO, RUNDC3A, SLC38A2, MAX.chr19.2478419.2478656, PDZD2, LOC100129726, CUX1, ANXA2, RXRA, S1PR4_A, FNBP1, FAM78A, IER2, PNMAL2, and MOBKL2A.

4. The method of claim 1, wherein the one or more genes are selected from SRRM3, HCN2, SPTBN4 and TMC6_A.

5. The method of claim 1, wherein the DNA is treated with a reagent that modifies DNA in a methylation-specific manner.

6. The method of claim 5, wherein the reagent comprises one or more of a methylation-sensitive restriction enzyme, a methylation-dependent restriction enzyme, and a bisulfite reagent.

7. The method of claim 6, wherein the DNA is treated with a bisulfite reagent to produce bisulfite-treated DNA.

8. The method of claim 1, wherein the measuring comprises multiplex amplification.

9. The method of claim 1, wherein measuring the amount of at least one methylated marker gene comprises using one or more methods selected from the group consisting of methylation-specific PCR, quantitative methylation-specific PCR, methylation-specific DNA restriction enzyme analysis, quantitative bisulfite pyrosequencing, flap endonuclease assay, PCR-flap assay, and bisulfite genomic sequencing PCR.

10. The method of claim 1, wherein the sample comprises one or more of a plasma sample, a blood sample, or a tissue sample (e.g., pancreatic tissue).

11. The method of claim 1, wherein the set of primers for the selected one or more genes is recited in Table 3.

12. A method of characterizing a sample, comprising:
a) measuring an amount of at least one methylated marker gene in DNA from the sample, wherein the at least one methylated marker gene is one or more genes selected from Tables 1A and 2A;
b) measuring the amount of at least one reference marker in the DNA; and
c) calculating a value for the amount of the at least one methylated marker gene measured in the DNA as a percentage of the amount of the reference marker gene measured in the DNA, wherein the value indicates the amount of the at least one methylated marker DNA measured in the sample.

13. The method of claim 12, wherein the one or more genes are selected from ANXA2, CACNA1C_A, CDHR2, FBXL16_B, GP1BB_A, GP1BB_C, HCN2, HPCAL1, LOC100129726, MAX.chr17.77788758-77788971, PDZD2, PTPRN2, RASSF3, RTN2, RUNDC3A, RXRA, SLC38A2, SPTBN4, SRRM3, STX10_B, TMC6_A, TSPO, CUX1, FAM78A, FNBP1, IER2, MOBKL2A, PNMAL2, S1PR4_A, LGALS3, and MYO15B.

14. The method of claim 12, wherein the one or more genes are selected from SRRM3, HCN2, SPTBN4, TMC6_A, GP1BB_C, GP1BB_A, STX10_B, CACNA1C_A, CDHR2, PTPRN2, MAX.chr17.77788758.77788971, FBXL16_B, RTN2, HPCAL1, RASSF3, TSPO, RUNDC3A, SLC38A2, MAX.chr19.2478419.2478656, PDZD2, LOC100129726, CUX1, ANXA2, RXRA, S1PR4_A, FNBP1, FAM78A, IER2, PNMAL2, and MOBKL2A.

15. The method of claim 12, wherein the one or more genes are selected from SRRM3, HCN2, SPTBN4 and TMC6_A.

16. The method of claim 12, wherein the at least one reference marker comprises one or more reference marker selected from B3GALT6 DNA, ZDHHC1 DNA, R-actin DNA, and non-cancerous DNA.

17. The method of claim 12, wherein the sample comprises one or more of a plasma sample, a blood sample, or a tissue sample (e.g., pancreatic tissue).

18. The method of claim 12, wherein the DNA is extracted from the sample.

19. The method of claim 12, wherein the DNA is treated with a reagent that modifies DNA in a methylation-specific manner.

20. The method of claim 19, wherein the reagent comprises one or more of a methylation-sensitive restriction enzyme, a methylation-dependent restriction enzyme, and a bisulfite reagent.

21. The method of claim 20 wherein the DNA is treated with a bisulfite reagent to produce bisulfite-treated DNA.

22. The method of claim 20, wherein the modified DNA is amplified using a set of primers for the selected one or more genes.

23. The method of claim 22, wherein the set of primers for the selected one or more genes is recited in Table 3.

24. The method of claim 12 wherein measuring amounts of a methylated marker gene comprises using one or more of polymerase chain reaction, nucleic acid sequencing, mass spectrometry, methylation-specific nuclease, mass-based separation, and target capture.

25. The method of claim 24, wherein the measuring comprises multiplex amplification.

26. The method of claim 24, wherein measuring the amount of at least one methylated marker gene comprises using one or more methods selected from the group consisting of methylation-specific PCR, quantitative methylation-specific PCR, methylation-specific DNA restriction enzyme analysis, quantitative bisulfite pyrosequencing, flap endonuclease assay, PCR-flap assay, and bisulfite genomic sequencing PCR.

27. A method for characterizing a biological sample comprising:
(a) measuring a methylation level of a CpG site for one or more genes selected from Table 1A and 2A in a biological sample of a human individual through
treating genomic DNA in the biological sample with bisulfite;
amplifying the bisulfite-treated genomic DNA using a set of primers for the selected one or more genes; and
determining the methylation level of the CpG site by methylation-specific PCR, quantitative methylation-specific PCR, methylation-sensitive DNA restriction enzyme analysis, quantitative bisulfite pyrosequencing, or bisulfite genomic sequencing PCR;
(b) comparing the methylation level to a methylation level of a corresponding set of genes in control samples without PNET; and
(c) determining that the individual has PNET when the methylation level measured in the one or more genes is higher than the methylation level measured in the respective control samples.

28. The method of claim 27, wherein the one or more genes are selected from ANXA2, CACNA1C_A, CDHR2, FBXL16_B, GP1BB_A, GP1BB_C, HCN2, HPCAL1, LOC100129726, MAX.chr17.77788758-77788971, PDZD2, PTPRN2, RASSF3, RTN2, RUNDC3A, RXRA, SLC38A2, SPTBN4, SRRM3, STX10_B, TMC6_A, TSPO, CUX1, FAM78A, FNBP1, IER2, MOBKL2A, PNMAL2, S1PR4_A, LGALS3, and MYO15B.

29. The method of claim 27, wherein the one or more genes are selected from SRRM3, HCN2, SPTBN4, TMC6_A, GP1BB_C, GP1BB_A, STX10_B, CACNA1C_A, CDHR2, PTPRN2, MAX.chr17.77788758.77788971, FBXL16_B, RTN2, HPCAL1, RASSF3, TSPO, RUNDC3A, SLC38A2, MAX.chr19.2478419.2478656, PDZD2, LOC100129726, CUX1, ANXA2, RXRA, S1PR4_A, FNBP1, FAM78A, IER2, PNMAL2, and MOBKL2A.

30. The method of claim 27, wherein the one or more genes are selected from SRRM3, HCN2, SPTBN4 and TMC6_A.

31. The method of claim 27 wherein the set of primers for the selected one or more genes is recited in Table 3.

32. The method of claim 27, wherein the biological sample is a plasma sample, a blood sample, or a tissue sample (e.g., pancreatic tissue).

33. The method of claim 27, wherein the one or more genes is described by the genomic coordinates shown in Tables 1A and 2A.

34. The method of claim 27, wherein said CpG site is present in a coding region or a regulatory region.

35. The method of claim 27, wherein said measuring the methylation level a CpG site for one or more genes comprises a determination selected from the group consisting of determining the methylation score of said CpG site and determining the methylation frequency of said CpG site.

36. A method, comprising:
(a) measuring a methylation level of a CpG site for one or more genes selected from Tables 1A and 2A
in a biological sample of a human individual through
treating genomic DNA in the biological sample with bisulfite;
amplifying the bisulfite-treated genomic DNA using a set of primers for the selected one or more genes; and
determining the methylation level of the CpG site by methylation-specific PCR, quantitative methylation-specific PCR, methylation-sensitive DNA restriction enzyme analysis, quantitative bisulfite pyrosequencing, or bisulfite genomic sequencing PCR.

37. The method of claim 36, wherein the one or more genes are selected from ANXA2, CACNA1C_A, CDHR2, FBXL16_B, GP1BB_A, GP1BB_C, HCN2, HPCAL1, LOC100129726, MAX.chr17.77788758-77788971, PDZD2, PTPRN2, RASSF3, RTN2, RUNDC3A, RXRA, SLC38A2, SPTBN4, SRRM3, STX10_B, TMC6_A, TSPO, CUX1, FAM78A, FNBP1, IER2, MOBKL2A, PNMAL2, S1PR4_A, LGALS3, and MYO15B.

38. The method of claim 36, wherein the one or more genes are selected from SRRM3, HCN2, SPTBN4, TMC6_A, GP1BB_C, GP1BB_A, STX10_B, CACNA1C_A, CDHR2, PTPRN2, MAX.chr17.77788758.77788971, FBXL16_B, RTN2, HPCAL1, RASSF3, TSPO, RUNDC3A, SLC38A2, MAX.chr19.2478419.2478656, PDZD2, LOC100129726, CUX1, ANXA2, RXRA, S1PR4_A, FNBP1, FAM78A, IER2, PNMAL2, and MOBKL2A.

39. The method of claim 36, wherein the one or more genes are selected from SRRM3, HCN2, SPTBN4 and TMC6_A.

40. The method of claim 36 wherein the set of primers for the selected one or more genes is recited in Tables 1A and 2A.

41. The method of claim 36, wherein the biological sample is a plasma sample, a blood sample, or a tissue sample (e.g., pancreatic tissue).

42. The method of claim 36, wherein the one or more genes is described by the genomic coordinates shown in Tables 3.

43. The method of claim 36,
wherein if the biological sample is a tissue sample then the one or more genes is selected from:
ANXA2, CACNA1C_A, CDHR2, FBXL16_B, GP1BB_A, GP1BB_C, HCN2, HPCAL1, LOC100129726, MAX.chr17.77788758-77788971, PDZD2, PTPRN2, RASSF3, RTN2, RUNDC3A, RXRA, SLC38A2, SPTBN4, SRRM3, STX10_B, TMC6_A, TSPO, CUX1, FAM78A, FNBP1, IER2, MOBKL2A, PNMAL2, S1PR4_A, LGALS3, and MYO15B;
SRRM3, HCN2, SPTBN4, TMC6_A, GP1BB_C, GP1BB_A, STX10_B, CACNA1C_A, CDHR2, PTPRN2, MAX.chr17.77788758.77788971, FBXL16_B, RTN2, HPCAL1, RASSF3, TSPO, RUNDC3A, SLC38A2, MAX.chr19.2478419.2478656, PDZD2, LOC100129726, CUX1, ANXA2, RXRA, S1PR4_A, FNBP1, FAM78A, IER2, PNMAL2, and MOBKL2A; and
SRRM3, HCN2, SPTBN4 and TMC6_A;
wherein if the biological sample is a plasma sample then the one or more genes is selected from:
ANXA2, CACNA1C_A, CDHR2, FBXL16_B, GP1BB_A, GP1BB_C, HCN2, HPCAL1, LOC100129726, MAX.chr17.77788758-77788971, PDZD2, PTPRN2, RASSF3, RTN2, RUNDC3A, RXRA, SLC38A2, SPTBN4, SRRM3, STX10_B, TMC6_A, TSPO, CUX1, FAM78A, FNBP1, IER2, MOBKL2A, PNMAL2, S1PR4_A, LGALS3, and MYO15B; and
SRRM3, HCN2, SPTBN4 and TMC6_A.

44. The method of claim 36, wherein said measuring the methylation level a CpG site for one or more genes comprises a determination selected from the group consisting of determining the methylation score of said CpG site and determining the methylation frequency of said CpG site.

45. A method of screening for a PNET in a sample obtained from a subject, the method comprising:
1) assaying a methylation state of a DNA methylation marker comprising a chromosomal region having an annotation recited in Tables 1A and 2A, and
2) identifying the subject as having PNET when the methylation state of the marker is different than a methylation state of the marker assayed in a subject that does not have PNET.

46. The method of claim 45, wherein the one or more genes are selected from ANXA2, CACNA1C_A, CDHR2, FBXL16_B, GP1BB_A, GP1BB_C, HCN2, HPCAL1, LOC100129726, MAX.chr17.77788758-77788971, PDZD2, PTPRN2, RASSF3, RTN2, RUNDC3A, RXRA, SLC38A2, SPTBN4, SRRM3, STX10_B, TMC6_A, TSPO, CUX1, FAM78A, FNBP1, IER2, MOBKL2A, PNMAL2, S1PR4_A, LGALS3, and MYO15B.

47. The method of claim 45, wherein the one or more genes are selected from SRRM3, HCN2, SPTBN4, TMC6_A, GP1BB_C, GP1BB_A, STX10_B, CACNA1C_A, CDHR2, PTPRN2, MAX.chr17.77788758.77788971, FBXL16_B, RTN2, HPCAL1, RASSF3, TSPO, RUNDC3A, SLC38A2, MAX.chr19.2478419.2478656, PDZD2, LOC100129726, CUX1, ANXA2, RXRA, S1PR4_A, FNBP1, FAM78A, IER2, PNMAL2, and MOBKL2A.

48. The method of claim 45, wherein the one or more genes are selected from SRRM3, HCN2, SPTBN4 and TMC6_A.

49. The method of claim 45 comprising assaying a plurality of markers.

50. The method of claim 45 wherein the marker is in a high CpG density promoter.

51. The method of claim 45 wherein the sample is a stool sample, a tissue sample, a pancreatic tissue sample, a plasma sample, or a urine sample.

52. The method of claim 45 wherein the assaying comprises using methylation specific polymerase chain reaction, nucleic acid sequencing, mass spectrometry, methylation specific nuclease, mass-based separation, or target capture.

53. The method of claim 45 wherein the assaying comprises use of a methylation specific oligonucleotide.

54. The method of claim 45,
wherein if the biological sample is a tissue sample then the one or more genes is selected from ANXA2, CACNA1C_A, CDHR2, FBXL16_B, GP1BB_A, GP1BB_C, HCN2, HPCAL1, LOC100129726, MAX.chr17.77788758-77788971, PDZD2, PTPRN2, RASSF3, RTN2, RUNDC3A, RXRA, SLC38A2, SPTBN4, SRRM3, STX10_B, TMC6_A, TSPO, CUX1, FAM78A, FNBP1, IER2, MOBKL2A, PNMAL2, S1PR4_A, LGALS3, and MYO15B;
wherein if the biological sample is a plasma sample then the one or more genes is selected from ANXA2, CACNA1C_A, CDHR2, FBXL16_B, GP1BB_A, GP1BB_C, HCN2, HPCAL1, LOC100129726, MAX.chr17.77788758-77788971, PDZD2, PTPRN2, RASSF3, RTN2, RUNDC3A, RXRA, SLC38A2, SPTBN4, SRRM3, STX10_B, TMC6_A, TSPO, CUX1, FAM78A, FNBP1, IER2, MOBKL2A, PNMAL2, S1PR4_A, LGALS3, and MYO15B.

55. A method for characterizing a sample from a human patient comprising:
a) obtaining DNA from a sample of a human patient;
b) assaying a methylation state of a DNA methylation marker comprising a chromosomal region having an annotation recited in Tables 1A and 2A;
c) comparing the assayed methylation state of the one or more DNA methylation markers with methylation level references for the one or more DNA methylation markers for human patients not having PNET.

56. The method of claim 55, wherein the one or more genes are selected from ANXA2, CACNA1C_A, CDHR2, FBXL16_B, GP1BB_A, GP1BB_C, HCN2, HPCAL1, LOC100129726, MAX.chr17.77788758-77788971, PDZD2, PTPRN2, RASSF3, RTN2, RUNDC3A, RXRA, SLC38A2, SPTBN4, SRRM3, STX10_B, TMC6_A, TSPO, CUX1, FAM78A, FNBP1, IER2, MOBKL2A, PNMAL2, S1PR4_A, LGALS3, and MYO15B.

57. The method of claim 55, wherein the one or more genes are selected from SRRM3, HCN2, SPTBN4, TMC6_A, GP1BB_C, GP1BB_A, STX10_B, CACNA1C_A, CDHR2, PTPRN2, MAX.chr17.77788758.77788971, FBXL16_B, RTN2, HPCAL1, RASSF3, TSPO, RUNDC3A, SLC38A2, MAX.chr19.2478419.2478656, PDZD2, LOC100129726, CUX1, ANXA2, RXRA, S1PR4_A, FNBP1, FAM78A, IER2, PNMAL2, and MOBKL2A.

58. The method of claim 55, wherein the one or more genes are selected from SRRM3, HCN2, SPTBN4 and TMC6_A.

59. The method of claim 55 wherein the sample is a stool sample, a tissue sample, a pancreatic tissue sample, a plasma sample, or a urine sample.

60. The method of claim 55 comprising assaying a plurality of DNA methylation markers.

61. The method of claim 55,
wherein if the biological sample is a tissue sample then the one or more genes is selected from ANXA2, CACNA1C_A, CDHR2, FBXL16_B, GP1BB_A, GP1BB_C, HCN2, HPCAL1, LOC100129726, MAX.chr17.77788758-77788971, PDZD2, PTPRN2, RASSF3, RTN2, RUNDC3A, RXRA, SLC38A2, SPTBN4, SRRM3, STX10_B, TMC6_A, TSPO, CUX1, FAM78A, FNBP1, IER2, MOBKL2A, PNMAL2, S1PR4_A, LGALS3, and MYO15B;
wherein if the biological sample is a plasma sample then the one or more genes is selected from ANXA2, CACNA1C_A, CDHR2, FBXL16_B, GP1BB_A, GP1BB_C, HCN2, HPCAL1, LOC100129726, MAX.chr17.77788758-77788971, PDZD2, PTPRN2, RASSF3, RTN2, RUNDC3A, RXRA, SLC38A2, SPTBN4, SRRM3, STX10_B, TMC6_A, TSPO, CUX1, FAM78A, FNBP1, IER2, MOBKL2A, PNMAL2, S1PR4_A, LGALS3, and MYO15B.

62. The method of claim 55 wherein the assaying comprises using methylation specific polymerase chain reaction, nucleic acid sequencing, mass spectrometry, methylation specific nuclease, mass-based separation, or target capture.

63. The method of claim 55 wherein the assaying comprises use of a methylation specific oligonucleotide.

64. The method of claim 63 wherein the methylation specific oligonucleotide is selected from a set of primers for the selected one or more genes recited in Table 3.

65. A method for characterizing a sample obtained from a human subject, the method comprising reacting a nucleic acid comprising a DMR with a bisulfite reagent to produce a bisulfite-reacted nucleic acid; sequencing the bisulfite-reacted nucleic acid to provide a nucleotide sequence of the bisulfite-reacted nucleic acid; comparing the nucleotide sequence of the bisulfite-reacted nucleic acid with a nucleotide sequence of a nucleic acid comprising the DMR from a subject who does not have PNET to identify differences in the two sequences.

66. A system for characterizing a sample obtained from a human subject, the system comprising an analysis component configured to determine the methylation state of a sample, a software component configured to compare the methylation state of the sample with a control sample or a reference sample methylation state recorded in a database, and an alert component configured to determine a single value based on a combination of methylation states and alert a user of a PNET-associated methylation state.

67. The system of claim 66 wherein the sample comprises a nucleic acid comprising a DMR.

68. The system of claim 66 further comprising a component for isolating a nucleic acid.

69. The system of claim 66 further comprising a component for collecting a sample.

70. The system of claim 66 wherein the sample is a stool sample, a tissue sample, a pancreatic tissue sample, a plasma sample, or a urine sample.

71. The system of claim 66 wherein the database comprises nucleic acid sequences comprising a DMR.

72. The system of claim 66 wherein the database comprises nucleic acid sequences from subjects who do not have PNET.

73. A kit comprising:
1) a bisulfite reagent; and
2) a control nucleic acid comprising a sequence from a DMR selected from a group consisting of DMR 1-198 from Table 1A and 2A, and having a methylation state associated with a subject who does not have PNET.

74. A kit comprising a bisulfite reagent and an oligonucleotide according to SEQ ID NOS 1-66.

75. A kit comprising a sample collector for obtaining a sample from a subject; reagents for isolating a nucleic acid from the sample; a bisulfite reagent; and an oligonucleotide according to SEQ ID NOS 1-66.

76. The kit according to claim 75 wherein the sample is a stool sample, a tissue sample, a pancreatic tissue sample, a plasma sample, or a urine sample.

77. A composition comprising a nucleic acid comprising a DMR and a bisulfite reagent.

78. A composition comprising a nucleic acid comprising a DMR and an oligonucleotide according to SEQ ID NOS 1-66.

79. A composition comprising a nucleic acid comprising a DMR and a methylation-sensitive restriction enzyme.

80. A composition comprising a nucleic acid comprising a DMR and a polymerase.

81. A method for screening for PNET in a sample obtained from a subject, the method comprising reacting a nucleic acid comprising a DMR with a bisulfite reagent to produce a bisulfite-reacted nucleic acid; sequencing the bisulfite-reacted nucleic acid to provide a nucleotide sequence of the bisulfite-reacted nucleic acid; comparing the nucleotide sequence of the bisulfite-reacted nucleic acid with a nucleotide sequence of a nucleic acid comprising the DMR from a subject who does not have PNET to identify differences in the two sequences; and identifying the subject as having PNET when a difference is present.

82. A system for screening for PNET in a sample obtained from a subject, the system comprising an analysis component configured to determine the methylation state of a sample, a software component configured to compare the methylation state of the sample with a control sample or a reference sample methylation state recorded in a database, and an alert component configured to determine a single value based on a combination of methylation states and alert a user of a PNET-associated methylation state.

83. The system of claim 82 wherein the sample comprises a nucleic acid comprising a DNA methylation marker comprising a base in a differentially methylated region (DMR) selected from a group consisting of DMR 1-198 from Tables 1A and 2A.

84. The system of claim 82 further comprising a component for isolating a nucleic acid.

85. The system of claim 82 further comprising a component for collecting a sample.

86. The system of claim 82 further comprising a component for collecting a stool sample, a pancreatic tissue sample, and/or a plasma sample.

87. The system of claim 82 wherein the database comprises nucleic acid sequences from subjects who do not have PNET.

88. A method for characterizing a biological sample comprising:
measuring a methylation level of a CpG site for one or more of SRRM3, HCN2, SPTBN4 and TMC6_A in a biological sample of a human individual through
treating genomic DNA in the biological sample with bisulfite;
amplifying the bisulfite-treated genomic DNA using primers specific for a CpG site for SRRM3, primers specific for a CpG site for HCN2, primers specific for a CpG site for SPTBN4, and primers specific for a CpG site for TMC6_A,
wherein the primers specific for SRRM3 are capable of binding an amplicon bound by SEQ ID Nos: 39 and 40, wherein the amplicon bound by SEQ ID Nos: 39 and 40 is at least a portion of a genetic region comprising chromosome 7 coordinates 75896582-75896785,
wherein the primers specific for HCN2 are capable of binding an amplicon bound by SEQ ID Nos: 13 and 14, wherein the amplicon bound by SEQ ID Nos: 13 and 14 is at least a portion of a genetic region comprising chromosome 19 coordinates 591692-591781,
wherein the primers specific for SPTBN4 are capable of binding an amplicon bound by SEQ ID Nos: 37 and 38, wherein the amplicon bound by SEQ ID Nos: 37 and 38 is at least a portion of a genetic region comprising chromosome 19 coordinates 41060185-41060270; and
wherein the primers specific for TMC6_A are capable of binding an amplicon bound by SEQ ID Nos: 43 and 44, wherein the amplicon bound by SEQ ID Nos: 43 and 44 is at least a portion of a genetic region comprising chromosome 17 coordinates 76123640-76123768;
determining the methylation level of the CpG site for the one or more of SRRM3, HCN2, SPTBN4 and TMC6_A by methylation-specific PCR, quantitative methylation-specific PCR, methylation-sensitive DNA restriction enzyme analysis, quantitative bisulfite pyrosequencing, or bisulfite genomic sequencing PCR.

89. The method of claim 88, wherein the biological sample is a blood sample or a pancreatic tissue sample.

90. The method of claim 88, wherein said CpG site is present in a coding region or a regulatory region.

91. The method of claim 88, wherein said measuring the methylation level of the CpG site for the one or more of SRRM3, HCN2, SPTBN4 and TMC6_A comprises a determination selected from the group consisting of determining the methylation score of said CpG site and determining the methylation frequency of said CpG site.

92. A method for characterizing a biological sample comprising:
measuring a methylation level of a CpG site for one or more markers selected from ANXA2, CACNA1C_A, CDHR2, FBXL16_B, GP1BB_A, GP1BB_C, HCN2, HPCAL1, LOC100129726, MAX.chr17.77788758-77788971, PDZD2, PTPRN2, RASSF3, RTN2, RUNDC3A, RXRA, SLC38A2, SPTBN4, SRRM3, STX10_B, TMC6_A, TSPO, CUX1, FAM78A, FNBP1, IER2, MOBKL2A, PNMAL2, S1PR4_A, LGALS3, and MYO15B in a biological sample of a human individual through
treating genomic DNA in the biological sample with bisulfite;
amplifying the bisulfite-treated genomic DNA using primers specific for a CpG site for the one or more markers,
wherein the primers specific for each marker are capable of binding an amplicon bound by the respective primer sequences recited in Table 3, wherein the amplicon bound by the respective primer sequences is at least a portion of a genetic region comprising the respective chromosomal region recited in Table 1A or Table 2A;
determining the methylation level of the CpG site for the one or more markers by methylation-specific PCR, quantitative methylation-specific PCR, methylation-sensitive DNA restriction enzyme analysis, quantitative bisulfite pyrosequencing, or bisulfite genomic sequencing PCR.

93. The method of claim 92, wherein the biological sample is a blood sample or a pancreatic tissue sample.

94. The method of claim 92, wherein said CpG site is present in a coding region or a regulatory region.

95. The method of claim 92, wherein said measuring the methylation level of the CpG site for the one or more markers comprises a determination selected from the group consisting of determining the methylation score of said CpG site and determining the methylation frequency of said CpG site.

96. A method for preparing a deoxyribonucleic acid (DNA) fraction from a biological sample of a human individual useful for analyzing one or more genetic loci involved in one or more chromosomal aberrations, comprising:
(a) extracting genomic DNA from a biological sample of a human individual;
(b) producing a fraction of the extracted genomic DNA by:
(i) treating the extracted genomic DNA with bisulfite;
(ii) amplifying the bisulfite-treated genomic DNA using separate primers specific for CpG sites for one or more markers recited in Tables 1A and 2A;
(c) analyzing one or more genetic loci in the produced fraction of the extracted genomic DNA by measuring a methylation level of the CpG site for each of the one or more markers.

97. The method of claim 96, wherein measuring a methylation level of the CpG site for each of the one or more markers is determined by methylation-specific PCR, quantitative methylation-specific PCR, methylation-sensitive DNA restriction enzyme analysis, or bisulfite genomic sequencing PCR.

98. The method of claim 96, wherein amplifying the bisulfite-treated genomic DNA using primers specific for a CpG site for each of the one or more markers is a set of primers that specifically binds at least a portion of a genetic region for the marker as shown in Tables 1A and/or 2A.

99. The method of claim 96, wherein the biological sample is a stool sample, a tissue sample, an organ secretion sample, a CSF sample, a saliva sample, a blood sample, a plasma sample, or a urine sample.

100. The method of claim 96, wherein each of the analyzed one or more genetic loci is associated with a PNET.

101. The method of claim 96, wherein the one or more markers are selected from ANXA2, CACNA1C_A, CDHR2, FBXL16_B, GP1BB_A, GP1BB_C, HCN2, HPCAL1, LOC100129726, MAX.chr17.77788758-77788971, PDZD2, PTPRN2, RASSF3, RTN2, RUNDC3A, RXRA, SLC38A2, SPTBN4, SRRM3, STX10_B, TMC6_A, TSPO, CUX1, FAM78A, FNBP1, IER2, MOBKL2A, PNMAL2, S1PR4_A, LGALS3, and MYO15B.

102. The method of claim 96, wherein the one or more markers are selected from SRRM3, HCN2, SPTBN4, TMC6_A, GP1BB_C, GP1BB_A, STX10_B, CACNA1C_A, CDHR2, PTPRN2, MAX.chr17.77788758.77788971, FBXL16_B, RTN2, HPCAL1, RASSF3, TSPO, RUNDC3A, SLC38A2, MAX.chr19.2478419.2478656, PDZD2, LOC100129726, CUX1, ANXA2, RXRA, S1PR4_A, FNBP1, FAM78A, IER2, PNMAL2, and MOBKL2A1.

103. The method of claim 96, wherein the one or more markers are selected from SRRM3, HCN2, SPTBN4 and TMC6_A.

104. A method for preparing a deoxyribonucleic acid (DNA) fraction from a biological sample of a human individual useful for analyzing one or more DNA fragments involved in one or more chromosomal aberrations, comprising:
(a) extracting genomic DNA from a biological sample of a human individual;
(b) producing a fraction of the extracted genomic DNA by:
(i) treating the extracted genomic DNA with bisulfite;
(ii) amplifying the bisulfite-treated genomic DNA using separate primers specific for CpG sites for one or more markers recited in Tables 1A and 2A;
(c) analyzing one or more DNA fragments in the produced fraction of the extracted genomic DNA by measuring a methylation level of the CpG site for each of the one or more markers.

105. The method of claim 104, wherein measuring a methylation level of the CpG site for each of the one or more markers is determined by methylation-specific PCR, quantitative methylation-specific PCR, methylation-sensitive DNA restriction enzyme analysis, or bisulfite genomic sequencing PCR.

106. The method of claim 104, wherein amplifying the bisulfite-treated genomic DNA using primers specific for a CpG site for each of the one or more markers is a set of primers that specifically binds at least a portion of a genetic region for the marker as shown in Tables 1A and/or 2A.

107. The method of claim 104, wherein the biological sample is a stool sample, a tissue sample, an organ secretion sample, a CSF sample, a saliva sample, a blood sample, a plasma sample, or a urine sample.

108. The method of claim 104, wherein each of the analyzed DNA fragments is associated with a PNET.

109. The method of claim 104, wherein the one or more markers are selected from ANXA2, CACNA1C_A, CDHR2, FBXL16_B, GP1BB_A, GP1BB_C, HCN2, HPCAL1, LOC100129726, MAX.chr17.77788758-77788971, PDZD2, PTPRN2, RASSF3, RTN2, RUNDC3A, RXRA, SLC38A2, SPTBN4, SRRM3, STX10_B, TMC6_A, TSPO, CUX1, FAM78A, FNBP1, IER2, MOBKL2A, PNMAL2, S1PR4_A, LGALS3, and MYO15B.

110. The method of claim 104, wherein the one or more markers are selected from SRRM3, HCN2, SPTBN4, TMC6_A, GP1BB_C, GP1BB_A, STX10_B, CACNA1C_A, CDHR2, PTPRN2, MAX.chr17.77788758.77788971, FBXL16_B, RTN2, HPCAL1, RASSF3, TSPO, RUNDC3A, SLC38A2, MAX.chr19.2478419.2478656, PDZD2, LOC100129726, CUX1, ANXA2, RXRA, S1PR4_A, FNBP1, FAM78A, IER2, PNMAL2, and MOBKL2A1.

111. The method of claim 104, wherein the one or more markers are selected from SRRM3, HCN2, SPTBN4 and TMC6_A.
